(19) United States
(12) Patent Application Publication
(10) Pub. No.: US 2023/0040280 A1
(43) Pub. Date: Feb. 9, 2023

(54) ANTISENSE OLIGONUCLEOTIDES FOR TREATMENT OF CANCER STEM CELLS

(71) Applicant: Andes Biotechnologies Global, Inc., Burlingame, CA (US)

(72) Inventors: Luis O. Burzio Eriz, Santiago (CL); Veronica A. Burzio Menendez, Santiago (CL); Jaime E. Villegas Olavarria, Santiago (CL)

(21) Appl. No.: 17/705,166

(22) Filed: Mar. 25, 2022

Related U.S. Application Data
(63) Continuation of application No. 16/573,983, filed on Sep. 17, 2019, now Pat. No. 11,319,535.
Application No. 16/573,983 is a continuation of application No. 15/826,535, filed on Nov. 29, 2017, now Pat. No. 10,457,943.
Application No. 15/826,535 is a continuation of application No. 14/775,654, filed on Sep. 11, 2015, now Pat. No. 9,862,944.
(60) Provisional application No. 61/937,436, filed on Feb. 7, 2014.
(60) Provisional application No. 61/790,072, filed on Mar. 15, 2013.
(60) Provisional application No. 61/785,269, filed on Mar. 14, 2013.

(51) Int. Cl. C12N 15/113; C12N 5/09; A61K 31/7088; A61K 45/06; A61K 31/7105

(52) U.S. Cl. CPC C12N 15/113; C12N 5/0693; A61K 31/7088; A61K 45/06; A61K 31/7105; C12N 2501/65; C12N 2501/999; C12N 2510/00; C12N 2310/113; C12N 2320/31; C12N 2310/11; C12N 2320/30

(57) ABSTRACT

The invention provides oligonucleotides complementary to a non-coding chimeric mitochondrial RNA as well as compositions and kits comprising the same, and their use in treating and preventing metastasis or relapse of a cancer in an individual previously treated for cancer with a therapy. The invention also provides oligonucleotides complementary to a non-coding chimeric mitochondrial RNA as well as compositions and kits comprising the same, and their use in treating a refractory cancer (e.g., a refractory HPV-associated cancer).

### CROSS-REFERENCE TO RELATED APPLICATIONS

This application claims the priority benefit of U.S. Provisional Patent Application Ser. No. 61/785,269, filed Mar. 14, 2013, U.S. Provisional Patent Application Ser. No. 61/790,072, filed Mar. 15, 2013, and U.S. Provisional Patent Application Ser. No. 61/937,438, filed Feb. 7, 2014, the entire content of each of which is incorporated herein by reference.

### FIELD OF THE INVENTION

This invention relates to oligonucleotides complementary to a non-coding chimeric mitochondrial RNA and their use in methods of treating and preventing metastasis or relapse of a cancer in an individual previously treated for cancer. The invention also relates to oligonucleotides complementary to a non-coding chimeric mitochondrial RNA and their use in treating a refractory cancer (e.g., a refractory HPV-associated cancer) in an individual.

### BACKGROUND OF THE INVENTION

Cancer is a cellular malignancy whose unique trait, loss of normal control of cell cycle, results in unregulated growth, lack of differentiation, and ability to invade other tissues and metastasize. Carcinogenesis is a multi-step process by which a normal cell is transformed in a malignant cell (McKinnell et al., “The Biology Basis of Cancer”, Ch. 3, 1998). The etiology of cancer is complex and includes alteration of the cell cycle regulation, chromosomal abnormalities and chromosomes breakage. Infectious agents (e.g., oncogenic viruses), chemicals, radiation (e.g., ultraviolet or ionizing radiation) and immunological disorders are thought to be the major causes of carcinogenesis (McKinnell et al., “The Biological Basis of Cancer, Ch.3, 1998).

Recent evidence supports the view that tumors are organized in a hierarchy of heterogeneous cell populations with different biological properties. Two models have been proposed to account for this heterogeneity within tumors and for tumor growth. One such model is based on cancer stem cells (CSCs) which are thought to be responsible for aspects of cancer such as initiation, progression, metastasis, and recurrence. See Chen et al., Acta Pharmacol Sin.,34(6):732-740, 2013; Ponti et al., Cancer Res, 65(13):5506-11, 2005; Singh et al., Cancer Res, 63:5821-5828, 2003; and Feng et al., Oncology Reports, 22:1129-1134, 2009. Although CSCs generally represent a very small population of the overall tumor population, they are generally regarded as a self-renewing initiation subpopulation of tumor cells or a small population of cancer cells that are capable of giving rise to new tumors. CSCs have been identified in a number of cancers including, but not limited to, breast, brain, blood, liver, kidney, cervical, ovarian, colon, and lung cancers among others. See Ponti et al., Cancer Res, 65(13):5506-11, 2005; Feng et al., Oncology Reports, 22:1129-1134, 2009; Zhang et al., Cancer Res, 68(11):4311:4320, 2008; Singh et al., Cancer Res, 63:5821-5828, 2003; Clarke et al., Cancer Res, 66:9339, 2006; Sendurai et al., Cell, 133:704, 2008; Ohata et al., Cancer Res, 72:5101, 2012; and Mukhopapadhyay et al., Plos One, 8(11);e78725, 2013).

Surgical resection of tumor(s) or metastases arising from a primary tumor(s) followed by systemic administration of anti-cancer therapy is the established clinical protocol for treatment of several cancers. Although successful for treatment of some cancer types, a well-known complication of cancer treatment is the survival of residual tumor cells or CSCs that are not effectively removed which can result in relapse after remission, with the cancer returning at the primary site of tumor formation or at distant sites due to metastasis. Recently, it was found that CSCs may also contribute to relapse after remission due to resistance to chemotherapy. See Domingo-Domenech et al., Cancer Cell, 22(3):373, 2012. Therefore, there is a need for development of therapeutic agents that can target cells which contribute to relapse and metastasis (e.g., CSCs). Discovery of such therapeutic agents may allow for the development of treatment useful for preventing recurrence of cancer after remission (i.e., relapse) or preventing the spread of the primary tumour to secondary sites (i.e., metastasis). See Clarke et al., Cancer Res, 66:9339, 2006.

All references cited herein, including patent applications, patent publications, and scientific literature, are herein incorporated by reference as if each individual reference were specifically and individually indicated to be incorporated by reference.

### SUMMARY OF THE INVENTION

The invention provided herein discloses, inter alia, methods for suppressing metastasis of a cancer in an individual comprising administering to the individual an effective amount of one or more oligonucleotide complementary to an antisense non-coding chimeric mitochondrial RNA (ASncmtRNA) molecule or a sense non-coding chimeric mitochondrial RNA (SncmtRNA) molecule, wherein the oligonucleotide is able to hybridize with the chimeric mitochondrial RNA molecules to form a stable duplex, and wherein the individual has been previously treated for cancer with a therapy. In some embodiments, the oligonucleotide is sufficiently complementary to a human non-coding chimeric mitochondria( RNA molecule comprising: an antisense 16S mitochondrial ribosomal RNA covalently linked at its 5′ end to the 3′ end of a polynucleotide with an inverted repeat sequence or a sense 16S mitochondrial ribosomal RNA covalently linked at its 5′ end to the 3′ end of a polynucleotide with an inverted repeat sequence. In any of the embodiments herein, the oligonucleotide can be complementary to the ASncmtRNA molecule encoded by a nucleotide sequence selected from the group consisting of SEQ ID NO:4, SEQ ID NO:5, and SEQ ID NO:6. In any of the embodiments herein, the oligonucleotide can be at least 85% complementary to the ASncmtRNA molecule encoded by a nucleotide sequence selected from the group consisting of SEQ ID NO:4, SEQ ID NO:5, and SEQ ID NO:6. In any of the embodiments herein, the one or more oligonucleotide can comprise a nucleotide sequence selected from the group consisting of SEQ ID NOs:7-198. In some embodiments, the one or more oligonucleotide comprises a nucleic acid sequence selected from the group consisting of SEQ ID NOs:36, 197 and 198. In any of the embodiments herein, the oligonucleotide can be administered in combination with at least one anti-cancer agent. In a further embodiment, the at least one anti-cancer agent is selected from the group consisting of remicade, docetaxel, celecoxib, melphalan, dexamethasone, steroids, gemcitabine, cisplatinum, temozolomide, etoposide, cyclophosphamide, temodar, carboplatin, procarbazine, gliadel, tamoxifen, topotecan, methotrexate, gefitinib, ta.xol, taxotere, fluorouracil, leucovorin, irinotecan, xeloda, CPT-11, interferon alpha, pegylated interferon alpha, capecitabine, cisplatin, thiotepa, fludarabine, carboplatin, liposomal daunorubicin, cytarabine, doxetaxol, pacilitaxel, vinblastine, IL-2, GM-CSF, dacarbazine, vinorelbine, zoledronic acid, palmitronate, biaxin, busulphan, prednisone, bortezomib, bisphosphonate, arsenic trioxide, vincristine, doxorubicin, paclitaxel, ganciclovir, adriamycin, estrainustine sodium phosphate, sulindac, and etoposide. In some embodiments, the oligonucleotide and the at least one anti-cancer agent is administered sequentially. In some embodiments, the oligonucleotide and the at least one anti-cancer agent is administered simultaneously. In any of the embodiments herein, the oligonucleotide, can be administered in combination with a radiation therapy. In any of the embodiments herein, the oligonucleotide can be administered in combination with surgery. In any of the embodiments herein, the oligonucleotide can be administered in combination with an allogenic stem cell transplant therapy. In any of the embodiments herein, the oligonucleotide can be administered in combination with an autologous stem cell transplant therapy. In any of the embodiments herein, the individual may have been previously treated for cancer with a therapy comprising chemotherapy, radiation therapy, surgery, or combinations thereof. In any of the embodiments herein, the cancer in the individual may have relapsed after treatment with one or more of bortezomib, cyclophosphamide, dexamethasone, doxorubicin, interferon-alpha, lenalidomide, melphalan, pegylated interferon-alpha, prednisone, thalidomide, and vincristine. In any of the embodiments herein, wherein the cancer can be a solid cancer. In a further embodiment, the solid cancer is bladder cancer, brain cancer, breast cancer, cervical cancer, colon cancer, endometrial cancer, esophageal cancer, gastric cancer, liver and bile duct cancer, lung cancer, melanoma, oral cancer, ovarian cancer, pancreatic cancer, pharynx cancer, prostate cancer, renal cancer, testicular cancer, or thyroid cancer. In any of the embodiments herein, wherein the cancer can be a non-solid cancer. In a further embodiment, the non-solid cancer is multiple myeloma, leukemia, or lymphoma. In any of the embodiments herein, the oligonucleotide can reduce the number of cancer stem cells in the individual as compared to an individual not administered the oligonucleotide. In any of the embodiments herein, the oligonucleotide can inhibit tumor growth and/ or metastasis in the individual as compared to an individual not administered the oligonucleotide.

In one aspect, the invention provided herein discloses, methods for preventing relapse of cancer in an individual comprising administering to the individual an effective amount of one or more oligonucleotide complementary to an antisense non-coding chimeric mitochondrial RNA (ASncmtRNA) molecule or a sense non-coding chimeric mitochondrial RNA (SncmtRNA) molecule, wherein the oligonucleotide is able to hybridize with the chimeric mitochondrial RNA molecules to form a stable duplex, and wherein the individual has been previously treated for cancer with a therapy. In a further embodiment, the oligonucleotide is sufficiently complementary to a human non-coding chimeric mitochondrial RNA molecule comprising: an antisense 16S mitochondrial ribosomal RNA covalently linked at its 5′ end to the 3′ end of a polynucleotide with an inverted repeat sequence or a sense 16S mitochondrial ribosomal RNA covalently linked at its 5′ end to the 3′ end of a polynucleotide with an inverted repeat sequence. In any of the embodiments herein, the oligonucleotide can be complementary to the ASncmtRNA molecule encoded by a nucleotide sequence selected from the group consisting of SEQ ID NO:4, SEQ ID NO:5, and SEQ ID NO:6. In any of the embodiments herein, the oligonucleotide can be at least 85% complementary to the ASncmtRNA molecule encoded by a nucleotide sequence selected from the group consisting of SEQ ID NO:4, SEQ ID NO:5, and SEQ ID NO:6. In any of the embodiments herein, the one or more oligonucleotide can comprise a nucleotide sequence selected from the group consisting of SEQ NOs:7-198. :In some embodiments, the one or more oligonucleotide comprises a nucleic acid sequence selected from the group consisting of SEQ ID NOs:36, 197 and 198. In any of the embodiments herein, the oligonucleotide can be administered in combination with at least one anti-cancer agent. In a further embodiment, the at least one anti-cancer agent is selected from the group consisting of remicade, docetaxel, celecoxib, melphalan, dexamethasone, steroids, gemcitabine, cisplatinum, temozolomide, etoposide, cyclophosphamide, temodar, carboplatin, procarbazine, gliadel, tamoxifen, topotecan, methotrexate, gefitinib, taxol, taxotere, fluorouracil, leucovorin, irinotecan, xeloda, CPT-11, interferon alpha, pegylated interferon alpha, capecitabine, cisplatin, thiotepa, fludarabine, carboplatin, liposomal daunorubicin, cytarabine, doxetaxol, pacilitaxel, vinblastine, IL-2, GM-CSF, dacarbazine, vinorelbine, zoledronic acid, palmitronate, biaxin, busulphan, prednisone, bortezomib, bisphosphonate, arsenic trioxide, vincristine, doxorubicin, paclitaxel, ganciclovir, adriamycin, estrainustine sodium phosphate, sulindac, and etoposide. In some embodiments, the oligonucleotide and the at least one anti-cancer agent is administered sequentially. In some embodiments, the oligonucleotide and the at least one anti-cancer agent is administered simultaneously. In any of the embodiments herein, the oligonucleotide can be administered in combination with a radiation therapy. In any of the embodiments herein, the oligonucleotide can be administered in combination with surgery. In any of the embodiments herein, the oligonucleotide can be administered in combination with an allogenic stem cell transplant therapy. In any of the embodiments herein, the oligonucleotide can be administered in combination with an autologous stern cell transplant therapy. In any of the embodiments herein, the individual may have been previously treated for cancer with a therapy comprising chemotherapy, radiation therapy, surgery, or combinations thereof. In any of the embodiments herein, the cancer in the individual may have relapsed after treatment with one or more of bortezomib, cyclophosphamide, dexamethasone, doxorubicin, interferon-alpha, lenalidomide, melphalan, pegylated interferon-alpha, prednisone, thalidomide, and vincristine. In any of the embodiments herein, wherein the cancer can be a solid cancer. In a further embodiment, the solid cancer is bladder cancer, brain cancer, breast cancer, cervical cancer, colon cancer, endometrial cancer, esophageal cancer, gastric cancer, liver and bile duct cancer, lung cancer, melanoma, oral cancer, ovarian cancer, pancreatic cancer, pharynx cancer, prostate cancer, renal cancer, testicular cancer, or thyroid cancer. In any of the embodiments herein, wherein the cancer can be a non-solid cancer. In a further embodiment, the non-solid cancer is multiple myeloma, leukemia, or lymphoma. In any of the embodiments herein, the oligonucleotide can reduce the number of cancer stem cells in the individual as compared to an individual not administered the oligonucleotide. In any of the embodiments herein, the oligonucleotide can inhibit tumor growth and/or metastasis in the individual as compared to an individual not administered the oligonucleotide.

In yet another aspect, the invention provided herein discloses, methods for the treatment of metastatic cancer in an individual comprising administering to the individual an effective amount of one or more oligonucleotide complementary to an antisense non-coding chimeric mitochondrial RNA (ASncmtRNA) molecule or a sense non-coding chimeric mitochondrial RNA (SncmtRNA) molecule, wherein the oligonucleotide is able to hybridize with the chimeric mitochondrial RNA molecules to form a stable duplex, and wherein the individual has been previously treated for cancer with a therapy. In a further embodiment, the oligonucleotide is sufficiently complementary to a human non-coding chimeric mitochondrial RNA molecule comprising: an antisense 16S mitochondrial ribosomal RNA covalently linked at its 5′ end to the 3′ end of a polynucleotide with an inverted repeat sequence or a sense 16S mitochondrial ribosomal RNA covalently linked at its 5′ end to the 3′ end of a polynucleotide with an inverted repeat sequence. In any of the embodiments herein, the oligonucleotide can be complementary to the ASncmtRNA molecule encoded by a nucleotide sequence selected from the group consisting of SEQ ID NO:4, SEQ ID NO:5, and SEQ ID NO:6. In any of the embodiments herein, the oligonucleotide can be at least 85% complementary to the ASncmtRNA molecule encoded by a nucleotide sequence selected from the group consisting of SEQ ID NO:4, SEQ ID NO:5, and SEQ ID NO:6. In any of the embodiments herein, the one or more oligonucleotide can comprise a nucleotide sequence selected from the group consisting of SEQ ID NOs:7-198. In some embodiments, the one or more oligonucleotide comprises a nucleic acid sequence selected from the group consisting of SEQ ID NOs:36, 197 and 198. In any of the embodiments herein, the oligonucleotide can be administered in combination with at least one anti-cancer agent. In a further embodiment, the at least one anti-cancer agent is selected from the group consisting of remicade, docetaxel, celecoxib, melphalan, dexamethasone, steroids, gemcitabine, cisplatinum, temozolomide, etoposide, cyclophosphamide, temodar, carboplatin, procarbazine, gliadel, tamoxifen, topotecan, methotrexate, gefitinib, taxol, taxotere, fluorouracil, leucovorin, irinotecan, xeloda, CPT-11, interferon alpha, pegylated interferon alpha, capecitabine, cisplatin, thiotepa, fludarabine, carboplatin, liposomal daunorubicin, cytarabine, doxetaxol, pacilitaxel, vinblastine, IL-2, GM-CSF, dacarbazine, vinorelbine, zoledronic acid, palmitronate, biaxin, busulphan, prednisone, bortezomib, bisphosphonate, arsenic trioxide, vincristine, doxorubicin, paclitaxel, ganciclovir, adriamycin, estrainustine sodium phosphate, sulindac, and etoposide. In some embodiments, the oligonucleotide and the at least one anti-cancer agent is administered sequentially. In some embodiments, the oligonucleotide and the at least one anti-cancer agent is administered simultaneously. In any of the embodiments herein, the oligonucleotide can be administered in combination with a radiation therapy. In any of the embodiments herein, the oligonucleotide can be administered in combination with surgery. In any of the embodiments herein, the oligonucleotide can be administered in combination with an allogenic stem cell transplant therapy. In any of the embodiments herein, the oligonucleotide can be administered in combination with an autologous stem cell transplant therapy. In any of the embodiments herein, the individual may have been previously treated for cancer with a therapy comprising chemotherapy, radiation therapy, surgery, or combinations thereof. In any of the embodiments herein, the metastatic cancer in the individual may have relapsed after treatment with one or more of bortezomib, cyclophosphamide, dexamethasone, doxorubicin, interferon-alpha, lenalidomide, melphalan, pegylated interferon-alpha, prednisone, thalidomide, and vincristine. In any of the embodiments herein, wherein the cancer can be a solid cancer. In a further embodiment, the solid cancer is bladder cancer, brain cancer, breast cancer, cervical cancer, colon cancer, endometrial cancer, esophageal cancer, gastric cancer, liver and bile duct cancer, lung cancer, melanoma, oral cancer, ovarian cancer, pancreatic cancer, pharynx cancer, prostate cancer, renal cancer, testicular cancer, or thyroid cancer. In any of the embodiments herein, wherein the cancer can be a non-solid cancer. In a further embodiment, the non-solid cancer is multiple myeloma, leukemia, or lymphoma. In any of the embodiments herein, the oligonucleotide can reduce the number of cancer stem cells in the individual as compared to an individual not administered the oligonucleotide. In any of the embodiments herein, the oligonucleotide can inhibit tumor growth and/or metastasis in the individual as compared to an individual not administered the oligonucleotide.

In yet another aspect, the invention provided herein discloses, methods for the treatment of a refractory cancer (e.g., a refractory HPV-associated cancer) in an individual comprising administering to the individual an effective amount of one or more oligonucleotide complementary to an antisense non-coding chimeric mitochondrial RNA (ASncmtRNA) molecule or a sense non-coding chimeric mitochondrial RNA (SncmtRNA) molecule, wherein the oligonucleotide is able to hybridize with the chimeric mitochondrial RNA molecules to form a stable duplex. In a further embodiment, the oligonucleotide is sufficiently complementary to a human non-coding chimeric mitochondrial RNA molecule comprising: an antisense 16S mitochondrial ribosomal RNA covalently linked at its 5′ end to the 3′ end of a polynucleotide with an inverted repeat sequence or a sense 16S mitochondrial ribosomal RNA covalently linked at its 5′ end to the 3′ end of a polynucleotide with an inverted repeat sequence. In any of the embodiments herein, the oligonucleotide can be complementary to the ASncmtRNA molecule encoded by a nucleotide sequence selected from the group consisting of SEQ ID NO:4, SEQ ID NO:5, and SEQ ID NO:6. In any of the embodiments herein, the oligonucleotide can be at least 85% complementary to the ASncmtRNA molecule encoded by a nucleotide sequence selected from the group consisting of SEQ ID NO:4, SEQ ID NO:5, and SEQ ID NO:6. In any of the embodiments herein, the one or more oligonucleotide can comprise a nucleotide sequence selected from the group consisting of SEQ ID NOs:7-198. In some embodiments, the one or more oligonucleotide comprises a nucleic acid sequence selected from the group consisting of SEQ ID NOs:36, 197 and 198. In any of the embodiments herein, the oligonucleotide can be administered in combination with at least one anti-cancer agent. In some embodiments, the oligonucleotide and the at least one anti-cancer agent is administered sequentially. In some embodiments, the oligonucleotide and the at least one anti-cancer agent is administered simultaneously. In any of the embodiments herein, the oligonucleotide can be administered in combination with a radiation therapy. In any of the embodiments herein, the oligonucleotide can be administered in combination with surgery. In any of the embodiments herein, the oligonucleotide can reduce the number of cancer stein cells in the individual as compared to an individual not administered the oligonucleotide. In any of the embodiments herein, the oligonucleotide can inhibit tumor growth and/or metastasis in the individual as compared to an individual not administered the oligonucleotide.

In another aspect, the invention herein provides kits comprising one or more oligonucleotide complementary to an antisense non-coding chimeric mitochondrial RNA (ASncmtRNA) molecule or a sense non-coding chimeric mitochondrial RNA (SncmtRNA) molecule and instructions for practicing any method disclosed herein.

### BRIEF DESCRIPTION OF THE DRAWINGS

FIG. 1 depicts a general scheme of the experimental procedure and the assay utilized herein to measure the effect of antisense oligonucleotides targeted to ASncmtRNA on the number of spheres formed in colon cancer cells, based on the specific ability of cancer stem cells to form these spheroid bodies.

FIG. 2 depicts representative examples of spheres formed in primary colon tumor cancer cells following no treatment, treatment with only Lipofectamine, treatment with a control oligonucleotide (Control Oligo 154), or treatment with an antisense oligonucleotide (ASO) targeted to ASncmtRNA (ASO 1537S). Treatment with the ASO 1537S abolished the formation of spheres.

FIG. 3 depicts a quantification of the capacity of primary colon tumor cells to form spheres. Approximately 0.6% of the total number of cells seeded were able to form spheres. Cells transfected with ASO 1537S were unable to form spheres.

FIG. 4 depicts representative examples of spheres formed in cells from the HCT-116 colon cancer cell line following no treatment, treatment with only Lipotectamine, treatment with a control oligonucleotide (Control Oligo 154), or treatment with an antisense oligonucleotide targeted to ASncmtRNA (ASO 1107S). Treatment with ASO 1107S abolished the formation of spheres.

FIG. 5 depicts a quantification of the capacity of HCT-116 colon cancer cells to form spheres. Approximately 0.3% of the total number of cells seeded were able to form spheres. Cells transfected with ASO 1107S were unable to form spheres.

FIG. 6 depicts a general scheme of the experimental procedure and the assay utilized herein to measure the effect of antisense oligonucleotides targeted to ASncmtRNA on the number of spheres formed by the cervical cancer SiHa cell line and primary culture cells of cervical tumors. The assay is based on the ability of cancer stem cells to form spheres, also referred to herein as spheroid bodies.

FIG. 7 depicts representative examples of spheres formed by the cervical cancer SiHa cell line and primary culture cells of cervical tumors following no treatment (NT), treatment with a control oligonucleotide (Control Oligo 154 : ASO-C), or treatment with an antisense oligonucleotide targeted to ASncmtRNA (ASO 1537S) or treatment with 45 μM cisplatin (CISP). Treatment with the ASO 1537S abolished the formation of spheres. The CerCa 3 cells obtained from primary culture, which is infected with HPV 45, is resistant to treatment with cisplatin as compared to two other cells obtained from primary culture, which are infected with HPV 16.

FIG. 8 depicts quantification of sphere formation by the SiHa cell cell line and primary cervical tumor cells (CerCa 1, CerCa 2 and CerCa 3) with or without treatment. Cells transfected with ASO 1537S wer unable to form spheres. The CerCa 3 primary culture infected with HPV 45 was resistant to treatment with cisplatin but not to treatment with ASO

FIG. 9 depicts the absence of tumor relapse and the absence of metastatic nodules in the lungs and liver of mice treated with ASO 1560S (squares) but not Control Oligo 154 (circles) following the surgical removal of intradermal melanoma tumors.

FIG. 10 depicts the presence of tumor relapse metastatic black nodules in the lung and livers of the mice treated with Control Oligo 154 but not in the lungs and livers mice treated with ASO 1560S.

FIG. 11 depicts the absence of tumor relapse and complete survival of mice treated with ASO 1560S (triangles) but not Control ASO 154 (squares) following surgical removal of intradermal kidney carcinoma tumors.

FIG. 12 depicts the absence of relapse in and survival of mice treated with ASO 1560S (squares) but not Control ASO 154 (circles) following surgical removal of intradermal kidney carcinoma tumors.

FIG. 13 depicts the absence of tumor relapse and complete survival of mice treated with ASO 1560S but not Control ASO 154 following surgical removal of intradermal melanoma carcinoma tumors.

FIG. 14 depicts the absence of tumors and complete survival of mice treated with ASO 1560S but not Control ASO 154 following surgical removal of subcutaneous bladder carcinoma tumors, ip indicates intraperitoneal administration and iv indicates intravenous administration.

FIG. 15 depicts the reduction in tumors and increase in survival of Rag −/− mice treated with ASO 1537S but not Control ASO 154 following the removal of a human A375 melanoma tumor.

### DETAILED DESCRIPTION

The invention provided herein discloses, inter alia, compositions comprising one or more oligonucleotide complementary to an antisense non-coding chimeric mitochondrial RNA (ASncmtRNA) molecule or a sense non-coding chimeric mitochondrial RNA (SncmtRNA) molecule and uses thereof for suppressing metastasis of a cancer in an individual. In certain embodiments, the invention provides compositions comprising one or more oligonucleotide complementary to an ASncmtRNA molecule or a SncmtRNA molecule and uses thereof for treating or preventing relapse of a cancer in an individual. In certain embodiments, the invention provides compositions comprising one or more oligonucleotide complementary to an ASncmtRNA molecule or a SncmtRNA molecule and uses thereof for treating metastatic cancer in an individual. In certain embodiments, the invention provides compositions comprising one or more oligonucleotide complementary to an ASncmtRNA molecule or a SncmtRNA molecule and uses thereof for treating a refractory cancer (e.g., a refractory HPV-associated cancer) in an individual. In some of the embodiments herein, the individual has been previously treated for cancer with a therapy (e.g., chemotherapy, radiation therapy, surgery or combinations thereof).

### I. General Techniques

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as, Molecular Cloning: A Laboratory Manual, second edition (Sambrook et al., 1989) and Molecular Cloning: A Laboratory Manual, third edition (Sambrook and Russel, 2001), (jointly referred to herein as “Sambrook”); Current Protocols in Molecular Biology (F. M. Ausubel et al., eds., 1987, including supplements through 2001); PCR: The Polymerase Chain Reaction, (Tullis et al., eds., 1994); Harlow and Lane (1988) Antibodies, A Laboratory Manual, Cold Spring Harbor Publications, New York; Harlow and Lane (1999) Using Antibodies: A Laboratory Manua,l Cold Spring Harbor Laboratory Press, Cold Spring Harbor, N.Y. (jointly referred to herein as “Harlow and Lane”), Beaucage et al. eds., Current Protocols in Nucleic Acid Chemistry John Wiley & Sons, Inc., New York, 2000), Handbook of Experimental Immunology, 4th edition (D. M. Weir & C. C. Blackwell, eds., Blackwell Science Inc., 1987); and Gene Transfer Vectors for Mammalian Cells (J. M. Miller & M. P. Calos, eds., 1987). Other useful references include Harrison's Principles of Internal Medicine (McGraw Hill; J. Isseleacher et al., eds.), Dubois' Lupus Erythematosus (5th ed.; D. J. Wallace and B. H. Hahn, eds.

### II. Definitions

Before describing the present invention in detail, it is to be understood that this invention is not limited to particular compositions or biological systems, which can, of course, vary. It is also to be understood that the terminology used herein is for the purpose of describing particular embodiments only, and is not intended to be limiting.

As used herein, the singular form “a”, “an”, and “the” includes plural references unless indicated otherwise. Thus, for example, reference to “an oligonucleotide” optionally includes a combination of two or more such oligonucleotides, and the like.

It is understood that aspects and embodiments of the invention described herein include “comprising,” “consisting,” and “consisting essentially of” aspects and embodiments.

An “isolated” nucleic acid molecule (e.g., “isolated oligonucleotide”) is a nucleic acid molecule that is identified and separated from at least one contaminant nucleic acid molecule with which it is ordinarily associated in the natural source of the nucleic acid. An isolated nucleic acid molecule is other than in the form or setting in which it is found in nature. Isolated nucleic acid molecules therefore are distinguished from the nucleic acid molecule as it exists in natural cells.

As used herein, the term “oligonucleotide complementary to an antisense non-coding chimeric mitochondrial RNA” or “oligonucleotide complementary to a sense non-coding chimeric mitochondrial RNA” refers to a nucleic acid having sufficient sequence complementarity to a target antisense non-coding chimeric mitochondrial RNA or a target sense non-coding chimeric mitochondrial RNA, respectively. An oligonucleotide “sufficiently complementary” to a target antisense non-coding chimeric mitochondrial RNA or a target sense non-coding chimeric mitochondrial RNA means that the oligonucleotide has a sequence sufficient to hybridize with the chimeric mitochondrial RNA molecules to form a stable duplex.

The term “oligonucleotide” refers to a short polymer of nucleotides and/or nucleotide analogs. An “oligonucleotide composition” of the invention includes any agent, compound or composition that contains one or more oligonucleotides, and includes, e.g., compositions comprising both single stranded and/or double stranded (ds) oligonucleotides, including, e.g., single stranded RNA, single stranded DNA, DNA/DNA and RNA/DNA hybrid oligonucleotides, as well as derivatized/modified oligonucleotides thereof. Such “oligonucleotide compositions” may also include amplified oligonucleotide products, e.g., polymerase chain reaction (PCR) products. An “oligonucleotide compositions” of the invention may also include art-recognized compositions designed to mimic the activity of oligonucleotides, such as peptide nucleic acid (PNA) molecules.

The phrase “corresponds to” or “sequence corresponding to” as it relates to RNA described herein (e.g., ASncmtRNA), indicates that the RNA has a sequence that is identical to or substantially the same as an RNA, or an RNA encoded by an analogous DNA, described herein. For example, an ASncmtRNA that corresponds to SEQ ID NO:4 indicates that the ASncmtRNA has a sequence that is identical to or substantially the same as the RNA of SEQ ID NO:203 or the RNA encoded by the analogous DNA of SEQ ID NO:4.

“Percent (%) nucleic acid sequence identity” or “percent (%) complementary” with respect to a reference nucleotide sequence (e.g., SncmtRNA sequence or ASncmtRNA sequence) is defined as the percentage of nucleic acid residues in a candidate sequence (e.g., oligonucleotide sequence) that are identical with the nucleic acid residues in the reference nucleotide sequence, after aligning the sequences and introducing gaps, if necessary, to achieve the maximum percent sequence identity, and not considering any conservative substitutions as part of the sequence identity. Alignment for purposes of determining percent nucleic acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For example, the % nucleic acid sequence identity of a given nucleic acid sequence A to, with, or against a given nucleic acid sequence B (which can alternatively be phrased as a given nucleic acid sequence A that has or comprises a certain % nucleic acid sequence identity to, with, or against a given nucleic acid sequence B) is calculated as follows:

100 times the fraction X/Y

where X is the number of nucleic acid residues scored as identical matches by the sequence in that program's alignment of A and B, and where Y is the total number of nucleic acid residues in B. It will be appreciated that where the length of nucleic acid sequence A is not equal to the length of nucleic acid sequence B, the t nucleic acid sequence identity of A to B will not equal the % nucleic acid sequence identity of B to A.

A “disorder” or “disease” is any condition that would benefit from treatment with a substance/molecule or method of the invention. This includes chronic and acute disorders or diseases including those pathological conditions which predispose the mammal to the disorder in question. In one embodiment, the disorder or disease is cancer. In another embodiment, the disorder or disease is metastatic cancer.

The term “cancer” refers to or describes the physiological condition in mammals that is typically characterized by unregulated cell growth/proliferation. Examples of cancer include but are not limited to, lymphoma, blastoma, sarcoma, and leukemia.

The terms “metastatic cancer” or “cancer metastasis” refer to a primary cancer capable of metastasis or cancer which has spread (i.e., metastasized) from a primary cancer, primary cancerous tissue or primary cancerous cells (e.g., cancer stem cells) from one part of of the body to one or more other parts of the body to form a secondary cancer or secondary cancers. Metastatic cancer or cancer metastasis also refer to locally advanced cancer that has spread from a primary cancer to nearby tissue(s) or lymph node(s). Metastatic cancer includes tumors that are defined as being high grade and/or high stage, for example tumors with a Gleason score of 6 or higher in prostate cancer are more likely to metastasize, Metastatic cancer also refers to tumors defined by one or more molecular markers that correlate with the metastasis.

The terms “relapsed cancer”, “relapse of a cancer”, “cancer relapse”, or “tumor relapse” refer to the return or reappearance of cancer after a period of improvement. Typically the period of improvement is after administration of a therapy that resulted in the decrease of or disappearance of signs and symptoms of cancer. The period of improvement can be the decrease or disappearance of all signs and symptoms of cancer. The period of improvement can also be the decrease or disappearance of some, but not all, signs and symptoms of cancer. In some embodiments, the relapsed cancer is a cancer that has become unresponsive or partially unresponsive to a drug or a therapy. For example and without limitation, relapsed cancer includes cancer in patients whose first progression occurs in the absence of any treatment following successful treatment with a drug or a therapy; cancer in patients who progress on a treatment, or within 60 days of the treatment; and cancer in patients who progress while receiving treatment.

The terms “cancer stem cell”, “cancer stem cells” or “CSCs” as used herein refer to a subpopulation of tumor cells or cancer cells. Cancer stem cells possess characteristics associated with normal stem cells, such as the ability to give rise to different cell types found in a particular cancer or tumor. Cancer stem cells have the capacity to drive the production or formation of a tumor or tumors through self-renewal and/or differentiation. Cancer stem cells have been identified in a number of cancers including, but not limited to, breast, brain, blood, liver, kidney, cervical, ovarian, colon, and lung cancers among others.

As used herein, “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, preventing or suppressing metastasis, decreasing the rate of disease progression, amelioration or palliation of the disease state, and remission or improved prognosis. In some embodiments, oligonucleotides described herein are used to prevent or suppress metastasis. An individual is successfully “treated”, for example, using an oligonucleotide of the invention if the individual shows observable and/or measurable reduction in or absence of one or more of the following: reduction in the number of cancer cells or absence of the cancer cells; reduction in the tumor size; inhibition (i.e., slow to some extent and preferably stop) of cancer cell infiltration into peripheral organs including the spread of cancer into soft tissue and bone; inhibition (i.e., slow to some extent and preferably stop) of tumor metastasis; inhibition, to some extent, of tumor growth or tumor relapse: and/or relief to some extent, one or more of the symptoms associated with the specific cancer; reduced morbidity and mortality, and improvement in quality of life issues.

As used herein, the term “prevention” includes providing prophylaxis with respect to occurrence or recurrence of a disease in an individual. An individual may be predisposed to, susceptible to a disorder, or at risk of developing a disorder, but has not yet been diagnosed with the disorder. In some embodiments, oligonucleotides described herein are used to prevent or suppress metastasis.

As used herein, an individual “at risk” of developing a disorder may or may not have detectable disease or symptoms of disease, and may or may not have displayed detectable disease or symptoms of disease prior to the treatment methods described herein. “At risk” denotes that an individual has one or more risk factors, which are measurable parameters that correlate with development of cancer (e.g., metastatic cancer), as known in the art. An individual having one or more of these risk factors has a higher probability of developing the disorder than an individual without one or more of these risk factors.

An “individual” or “subject” can be a vertebrate, a mammal, or a human. Mammals include, but are not limited to, farm animals (such as cows), sport animals, pets (such as horses), primates, mice and rats. Individuals also include companion animals including, but not limited to, dogs and cats. In one aspect, an individual is a human.

A “healthcare professional,” as used herein, can include, without limitation, doctors, nurses, physician assistants, lab technicians, research scientists, clerical workers employed by the same, or any person involved in determining, diagnosing, aiding in the diagnosis or influencing the course of treatment for the individual.

An “effective amount” refers to an amount of therapeutic compound, such as an oligonucleotide or other anticancer therapy, administered to an individual, either as a single dose or as part of a series of doses, which is effective to produce a desired therapeutic or prophylactic result.

A “therapeutically effective amount” is at least the minimum concentration required to effect a measurable improvement of a particular disorder. A therapeutically effective amount herein may vary according to factors such as the disease state, age, sex, and weight of the patient, and the ability of the oligonucleotide to elicit a desired response in the individual. A therapeutically effective amount may also be one in which any toxic or detrimental effects of the oligonucleotide are outweighed by the therapeutically beneficial effects. In the case of cancer, the therapeutically effective amount of the oligonucleotide may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the oligonucleotide may prevent growth and/or kill existing cancer cells, it may be cytostatic and/or cytotoxic.

A “prophylactically effective amount” refers to an amount effective, at the dosages and for periods of time necessary, to achieve the desired prophylactic result. For example, a prophylactically effective amount of the oligonucleotides of the present invention is at least the minimum concentration that prevents or attenuates the development of at least one symptom of metastatic cancer.

Administration “in combination with” one or more further therapeutic agents includes simultaneous (concurrent) and consecutive administration in any order.

The term “pharmaceutical formulation” refers to a preparation that is in such form as to permit the biological activity of the active ingredient to be effective, and that contains no additional components that are unacceptably toxic to a subject to which the formulation would be administered. Such formulations are sterile.

A “sterile” formulation is aseptic or free from all living microorganisms and their spores.

The term “package insert” is used to refer to instructions customarily included in commercial packages of therapeutic products, that contain information about the indications, usage, dosage, administration, contraindications and/or warnings concerning the use of such therapeutic products.

The term “about” as used herein refers to the usual error range for the respective value readily known to the skilled person in this technical field.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### III. Oligonucleotides and Other Anti-Cancer Therapies

Human cells express a number of unique chimeric mitochondrial RNA molecules. These molecules are non-coding (i.e., they are not known to serve as a template for the translation of a protein) and comprise the 16S mitochondrial ribosomal RNA covalently linked at the 5′ end to an inverted repeat sequence. Chimeric mitochondrial RNA molecules are found in two forms: sense and antisense.

The sense chimeric non-coding mitochondrial RNA (SncmtRNA) molecule corresponds to the 16S mitochondrial ribosomal RNA transcribed from the “H-strand” of the circular mitochondria genome. Covalently linked to the 5′ end of this RNA molecule is a nucleotide sequence or inverted repeat sequence corresponding to an RNA transcribed from the “L-strand” of the mitochondrial 16S gene. The size of the inverted repeat sequence in the SncmtRNA can vary from about 25, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, or 800 nucleotides or more to between about 100-200, 150-250, 200-300, 250-350, 400-500, 450-550, 500-600, 550-650, 600-700, 650-750, or 700-800 nucleotides or more, including any number in between these values. In one embodiment, the inverted repeat sequence in the SncmtRNA corresponds to a fragment of 815 nucleotides of the RNA transcribed from the L-strand of the 16S gene of the mitochondrial genome. In another embodiment, the inverted repeat sequence in the SncmtRNA corresponds to a fragment of 754 nucleotides of the RNA transcribed from the L-strand of the 16S gene of the mitochondrial genome. In still another embodiment, the inverted repeat sequence in the SncmtRNA corresponds to a fragment of 694 nucleotides of the RNA transcribed from the L-strand of the 16S gene of the mitochondrial genome. In another embodiment, the SncmtRNA corresponds to SEQ ID NO:1, SEQ ID NO:2, or SEQ ID NO:3. In another embodiment, the SncmtRNA comprises a sequence selected from the group consisting of SEQ ID NO:200, SEQ ID NO:201, and SEQ ID NO:202.

The antisense chimeric non-coding mitochondrial RNA (ASncmtRNA) molecule corresponds to the 16S mitochondrial ribosomal RNA transcribed from the “L-strand” of the circular mitochondrial genome. Covalently linked to the 5′ end of this RNA molecule is a nucleotide sequence or the inverted repeat sequence corresponding to an RNA transcribed from the “H-strand” of the mitochondrial 16S gene. The size of the inverted repeat sequence in the ASncmtRNA can vary from about 25, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800 nucleotides or more to between about 100-200, 150-250, 200-300, 250-350, 400-500, 450-550, 500-600, 550-650, 600-700, 650-750, or 700-800 or more, including any number in between these values. In another embodiment, the ASncmtRNA corresponds to SEQ ID NO:4, SEQ ID NO:5, or SEQ ID NO:6. In another embodiment, the ASncmtRNA comprises a sequence selected from the group consisting of SEQ ID NO:203, SEQ ID NO:204, and SEQ ID NO:205.

Further information related to chimeric mitochondrial RNA molecules can be found in U.S. Pat. No. 8,318,686, the disclosure of which is incorporated by reference herein in its entirety.

In one aspect, the invention provides one or more oligonucleotide complementary to an ASncmtRNA molecule or a SncmtRNA molecule, wherein the oligonucleotide is able to hybridize with the chimeric mitochondrial RNA molecules to form a stable duplex for use in a method disclosed herein. In some aspects, provided herein are methods for suppressing metastasis of a cancer in an individual using one or more oligonucleotides described herein. In some aspects, provided herein are methods for treating or preventing relapse of a cancer in an individual. In some aspects, provided herein are methods for treating metastatic cancer in an individual. In some embodiments herein, the individual has been previously treated for cancer with a therapy (e.g., chemotherapy, radiation therapy, surgery or combinations thereof). In some embodiments, the one or more oligonucleotide complementary to an ASncmtRNA molecule or a SncmtRNA molecule described herein has or more of the following characteristics when used in a method disclosed herein; (1) hybridizes with the chimeric mitochondrial RNA molecules (i.e., an ASncmtRNA molecule or a SncmtRNA molecule) to form a stable duplex; (2) hybridizes with the chimeric mitochondrial RNA molecules expressed by tumor cells and inhibits, arrests, kills or abolishes tumor cells; (3) hybridizes with the chimeric mitochondrial RNA molecules expressed by cancer stem cells (CSCs) and inhibits, arrests, kills or abolishes CSCs; (4) suppresses metastasis of a cancer in an individual (e.g., an individual previously treated for cancer with a therapy); (5) treats or prevents relapse of a cancer in an individual (e.g., an individual previously treated for cancer with a therapy); (6) treats metastatic cancer in an individual (e.g., an individual previously treated for cancer with a therapy); and (7) prolongs overall survival in an individual previously treated for cancer with a therapy (e.g., chemotherapy, radiation therapy, surgery or combinations thereof).

In one aspect, the oligonucleotides for use in any of the methods described herein can be complementary to a SncmtRNA molecule and/or to an ASncmtRNA molecule disclosed herein. Without being bound to theory, it is believed that the complementary oligonucleotides bind to the ncmtRNAs and interfere with their cellular functions. As used herein, an oligonucleotide sequence is “complementary” to a portion of an ncmtRNA, as referred to herein, if the oligonucleotide possesses a sequence having sufficient complementarity to be able to hybridize with the ncmtRNA to form a stable duplex. The ability to hybridize will depend on both the degree of complementarity and the length of the oligonucleotide. Generally, the longer the hybridizing oligonucleotide, the more base mismatches with an ncmtRNA it may contain and still form a stable duplex. In some aspects, the one or more oligonucleotide used according to the methods disclosed herein is at least 8 (such as at least 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more) base pairs in length. Those skilled in the art can ascertain a tolerable degree of mismatch by use of standard procedures to determine the melting point of the hybridized complex. In some embodiments, the one or more oligonucleotide is at least 85% (such as at least 86%, 87%, 88%, 89%, 90%, 91%, 92%, 93%, 94%, 95%, 96%, 97%, 98%, 99%, or 100%) complementary to a SncmtRNA molecule and/or to a ASncmtRNA molecule disclosed herein. In some embodiments, the complementary oligonucleotide is an antisense oligonucleotide. In one embodiment, the one or more oligonucleotide is complementary to one or more ncmtRNA encoded by a nucleic acid sequence selected from the group consisting of SEQ ID NOs:1-6. In some embodiments, the one or more oligonucleotide comprises a nucleic acid sequence selected from the group consisting of SEQ ID NOs:7-198. In some embodiments, the one or more oligonucleotide comprises a nucleic acid sequence selected from the group consisting of SEQ ID NOs:36, 197 and 198. In some embodiments, the one or more oligonucleotide comprises a nucleic acid sequence selected from the group consisting of SEQ ID NOs:36, 197 and 198.

a. Oligonucleotide Modifications

The naturally occurring internucleoside linkage of RNA and DNA is a 3′ to 5 phosphodiester linkage. The oligonucleotides (e.g., an antisense oligonucleotide) used for suppressing metastasis of a cancer, treating or preventing relapse of a cancer, or treating metastatic cancer according to any of the methods disclosed herein can have one or more modified, i.e. non-naturally occurring, internucleoside linkages. With respect to therapeutics, modified internucleoside linkages are often selected over oligonucleotides having naturally occurring internucleoside linkages because of desirable properties such as, for example, enhanced cellular uptake, enhanced affinity for target nucleic acids, and increased stability in the presence of nucleases present in bodily fluids.

Oligonucleotides (e.g., an antisense oligonucleotide) having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known in the art.

In one embodiment, oligonucleotides (e.g., an antisense oligonucleotide) targeted to a SncmtRNA molecule and/or to an ASncmtRNA molecule disclosed herein comprise one or more modified internucleoside linkages. In some embodiments, the modified internucleoside linkages are phosphorothioate linkages.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

Specific, though nonlimiting, examples of oligonucleotides (e.g., an antisense oligonucleotide) useful in the methods of the present invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleotides.

In some embodiments, modified oligonucleotide backbones include, for example, phosphorothioates, chiral phosphorothioates, phosphorodithioates, phosphotriesters, aminoalkylphosphotri-esters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thiono-phosphoramidates, thionoalkylphosphonates, thionoalkylphospho-triesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof) can also be employed. Various salts, mixed salts and free acid forms are also included. Oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In other embodiments, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science. 254:1497-1500, 1991.

Representative United States patents that teach the preparation of the above phosphorus-containing and non-phosphorus-containing linkages include, but are not limited to, U.S. Pat. Nos. 5,541,306; 5,550,111; 5,563,253; 5,571,799; 5,587,361; 5,194,599; 5,565,555; 5,527,899; 5,721,218; 5,672,697 and 5,625,050, 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, each of which is herein incorporated by reference.

Modified oligonucleotides antisense oligonucleotides) complementary to SncmtRNA and/or ASncmtRNA used as anticancer therapies in combination with any of the methods disclosed herein (e.g., method of suppressing metastasis of a cancer) may also contain one or more substituted sugar moieties. For example, the furanosyl sugar ring can be modified in a number of ways including substitution with a substituent group, bridging to form a bicyclic nucleic acid “BNA” and substitution of the 4′-O with a heteroatom such as S or N(R) as described in U.S. Pat. No. 7,399,845, hereby incorporated by reference herein in its entirety. Other examples of BNAs are described in published. International Patent Application No. WO 2007/146511, hereby incorporated by reference herein in its entirety.

The oligonucleotides (e.g., antisense oligonucleotides) for use in the methods disclosed herein (e.g., method of suppressing metastasis of a cancer) can optionally contain one or more nucleotides having modified sugar moieties. Sugar modifications may impart nuclease stability, binding affinity or some other beneficial biological property to the antisense compounds. The furanosyl sugar ring of a nucleoside can be modified in a number of ways including, but not limited to: addition of a substituent group, particularly at the 2′ position; bridging of two non-geminal ring atoms to form a bicyclic nucleic acid (BNA); and substitution of an atom or group such as —S—, —N(R)— or —C(R1)(R2) for the ring oxygen at the 4′-position. Modified sugars include, but are not limited to: substituted sugars, especially 2′-substituted sugars having a 2′-F, 2′-OCH2 (2′-OMe) or a 2′-O (CH₂)₂—OCH₃ (2′-O-methoxyethyl or 2-MOE) substituent group; and bicyclic modified sugars (BNAs), having a 4′-(CH₂)n-O-2′ bridge, where n=1 or n=2. Methods for the preparations of modified sugars are well known to those skilled in the art.

In certain embodiments, a 2′-modified nucleoside has a bicyclic sugar moiety. In certain such embodiments, the bicyclic sugar moiety is a D sugar in the alpha configuration. In certain such embodiments, the bicyclic sugar moiety is a D sugar in the beta configuration. In certain such embodiments, the bicyclic sugar moiety is an L sugar in the alpha configuration. In certain such embodiments, the bicyclic sugar moiety is an L sugar in the beta configuration.

In other embodiments, the bicyclic sugar moiety comprises a bridge group between the 2′ and the 4′-carbon atoms. In certain such embodiments, the bridge group comprises from 1 to linked biradical groups. In certain embodiments, the bicyclic sugar moiety comprises from 1 to 4 linked biradical groups. In certain embodiments, the bicyclic sugar moiety comprises 2 or 3 linked biradical groups. In certain embodiments, the bicyclic sugar moiety comprises 2 linked biradical groups. In certain embodiments, a linked biradical group is selected from —O—, —S—, —N(R1)-, —C(R1)(R2)-, —C(R1)═C(R1)-, —C(R1)═N—, —C(═NR1)-, —Si(R1)(R2)-, —S(═O)2-, —S(═O)—, —C(═O)— and —C(═S)—; where each R1 and R2 is, independently, H, hydroxyl, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, a heterocycle radical, a substituted hetero-cycle radical, heteroaryl, substituted heteroaryl, C5-C7 alicyclic radical, substituted C5-C7 alicyclic radical, halogen, substituted oxy (—O—), amino, substituted amino, azido, carboxyl, substituted carboxyl, acyl, substituted acyl, CN, thiol, substituted thiol, sulfonyl (S(═O)2-H), substituted sulfonyl, sulfoxyl (S(═O) H) or substituted sulfoxyl; and each substituent group is, independently, halogen, C1-C12 alkyl, substituted C1-C12 alkyl, C2-C12 alkenyl, substituted C2-C12 alkenyl, C2-C12 alkynyl, substituted C2-C12 alkynlyl, amino, substituted amino, acyl, substituted acyl, aminoalkyl, C1-C12 aminoalkoxy, substituted C1-C12 aminoalkyl, substituted C1-C12 aminoalkoxy or a protecting group.

Oligonucleotides (e.g., antisense oligonucleotides) for use in any of the methods disclosed herein (e.g., method of suppressing metastasis of a cancer) may also include nucleobase (often referred to in the art simply as “base”) modifications or substitutions. Nucleobase modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications may impart nuclease stability, binding affinity or some other beneficial biological property to oligonucleotide compounds. Modified nucleobases include synthetic and natural nucleobases such as, for example, 5-methylcytosine (5-me-C). Certain nucleobase substitutions, including 5-methylcytosine substitutions, are particularly useful for increasing the binding affinity of an oligonucleotide compound (such as an antisense oligonucleotide compound) for a target nucleic acid (such as an ncmtRNA).

Additional unmodified nucleobases include 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

Heterocyclic base moieties may also include those in which the purine or pyrimidine base is replaced with other heterocycles, for example 7-deaza-adenine, 7-deazaguanosine, 2-aminopyridine and 2-pyridone. Nucleobases that are particularly useful for increasing the binding affinity of antisense compounds include 5-substituted pyrimidines, 6-azapyrimidines and N-2, N-6 and O-6 substituted purines, including 2 aminopromladenine, 5-propynyluracil and 5-propynylcytosine.

As used herein, “unmodified” or “natural” nucleobases include the purine bases adenine (A) and guanine (G), and the pyrimidine bases thymine (T), cytosine (C) and uracil (U).

Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido[5,4-b ][1,4]benzoxazin-2(3H)-one), phenothiazine cytidine (1H-pyrimido[5,4-b][1,4]benzothiazin-2(3H)-one), O-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B. ed., CRC Press, 1993.

Representative United States patents that teach the preparation of certain of the above noted modified nucleobases as well as other modified nucleobases include, but are not limited to, U.S. Pat. Nos. 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; and 5,681,941, each of which is herein incorporated by reference.

b. Ribozymes

In another embodiment of the invention, ribozymes can be used to interfere with the ncmtRNA molecules described herein to induce cell death in proliferative cells associated with mestastasis (e.g., CSCs). The sequence of the ribozyme can be designed according to the sequence of the ASncmtRNA (for example, a sequence corresponding to SEQ ID NO:4, SEQ ID NO:5, or SEQ ID NO:6, or a sequence comprising SEQ ID NO:203, SEQ ID NO:204, or SEQ ID NO:205) or the SncmtRNA (for example, a sequence corresponding to SEQ ID NO:1, SEQ ID NO:2, or SEQ ID NO:3, or a sequence comprising SEQ ID NO:200, SEQ ID NO:202, or SEQ ID NO:203) to cleave specific regions of the transcript. Ribozymes are enzymatic RNA molecules capable of catalyzing the specific cleavage of RNA (Rossi, Curr. Biology 4:469-471, 1994). The mechanism of ribozyme action involves sequence specific hybridization of the ribozyme molecule to complementary target RNA, followed by an endonucleolytic cleavage. The composition of ribozyme molecules must include one or more sequences complementary to the RNA, and must include the well-known catalytic sequence responsible for RNA cleavage, and described in U.S. Pat. No. 5,093,246, the disclosure of which is incorporated by reference herein in its entirety. As such, within the scope of the invention, hammerhead ribozyme molecules can be engineered that specifically and efficiently catalyze endonucleolytic cleavage of the ASncmtRNA or SncmtRNA molecules disclosed herein. The construction and production of hammerhead ribozymes is well known in the art and it was described (Haseloff et al., Gene, 82:43-52, 1989). Ribozymes of the present invention can also include RNA endoribonucleases (Zaug et al., Science, 224:574-578, 1984). In some embodiments, a ribozyme described herein can be used in any method described herein. In some embodiments, a method of suppressing metastasis of a cancer in an individual comprises administering to the individual an effective amount of one or more ribozyme described herein. In some embodiments, a method for treating or preventing relapse of cancer in an individual comprises administering to the individual an effective amount of one or more ribozyme described herein. In some embodiments, a method for treating metastatic cancer in an individual comprises administering to the individual an effective amount of one or more ribozyme described herein. In some embodiments, a method for treating a refractory cancer (e.g., a refractory HPV-associated cancer) in an individual comprises administering to the individual an effective amount of one or more ribozyme described herein. In some embodiments, the individual has been previously treated for cancer with a therapy (e.g., chemotherapy, radiation therapy, surgery or combinations thereof).

c. RNA interference

In another aspect, interference with the function of the ASncmtRNA and/or SncmtRNA molecules disclosed herein for use in any of the methods disclosed herein (e.g., method of suppressing metastasis of a cancer) can be achieved by RNA interference or RNA silencing. RNA interference (RNAi) has emerged as a novel and promising approach for gene silencing in mammalian cells (Elbashir et al., Nature 411:494-498, 2001; McManus et al., Nature Rev. Genet. 3:737-747, 2002). Synthetically synthesized double stranded RNA molecules of about 8 to 40 (such as about 10 to 36, 14 to 32, 18-28, or 22-24) base pairs (bp) or at least about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 bp in length hybridize specifically to their complementary target RNA, leading to degradation of the RNA. Several different genes have been silenced successfully by small interfering RNA or siRNA (Lu et al., Curr. Opin. Mol. Ther. 5:225-234, 2003; Wacheck et al., Oligonucleotides 13:393-400, 2003). Therefore, synthetic double stranded RNA targeted to the ASncmtRNA and/or SncmtRNA molecules disclosed herein can be used to degrade these transcripts and induce cancer cell death (e.g., CSC death). Those familiar in the art will understand that the sequence of the siRNA has to be complementary to any region of the ASncmtRNA and/or SncmtRNA molecules (such as complementary to any one of a sequence corresponding to SEQ ID NO:1, SEQ ID NO:2, SEQ ID NO:3, SEQ ID NO:4, SEQ ID NO:5, and/or SEQ ID NO:6, or complementary to any one of a sequence comprising SEQ ID NO:200, SEQ ID NO:201, SEQ ID NO:202, SEQ ID NO:203, SEQ ID NO:204, and/or SEQ ID NO:205). In some embodiments, an RNA described herein can be used in any method described herein. In some embodiments, a method of suppressing metastasis of a cancer in an individual comprises administering to the individual an effective amount of one or more RNA described herein. In some embodiments, a method for treating or preventing relapse of cancer in an individual comprises administering to the individual an effective amount of one or more RNA described herein. In some embodiments, a method for treating metastatic cancer in an individual comprises administering to the individual an effective amount of one or more RNA described herein. In some embodiments, a method for treating a refractory cancer (e.g., a refractory HPV-associated cancer) in an individual comprises administering to the individual an effective amount of one or more RNA described herein. In some embodiments, the individual has been previously treated for cancer with a therapy (e.g., chemotherapy, radiation therapy, surgery or combinations thereof).

d. Oligonucleotide Delivery

In one embodiment, a recombinant vector can be used for delivering one or more oligonucleotides (such as any of the oligonucleotides disclosed herein) complementary to a sense and/or antisense chimeric non-coding mitochondrial RNA molecule to the individual. This can include both systemic delivery and delivery localized to a particular region of the body (such as, the bone marrow). Any vector capable of enabling recombinant production of one or more oligonucleotides complementary to a sense or antisense chimeric ncmtRNA molecule and/or which can deliver one or more oligonucleotides complementary to a sense or antisense chimeric ncmtRNA molecule into a host cell is contemplated herein. The vector can be either RNA or DNA, either prokaryotic or eukaryotic, and typically is a virus or a plasmid. The vector can be part of a DNA vaccine or used as part of any other method for delivering a heterologous gene for expression in a host cell that is known to one having skill in the art. Recombinant vectors are capable of replicating when transformed into a suitable host cell. Viral vectors infect a wide range of non-dividing human cells and have been used extensively in live vaccines without adverse side effects. A viral vector (such as, but not limited to, an adenoviral vector or an adeno-associated viral (AAV) vector (e.g. AAV-1, AAV-2, AAV-3, AAV-4, AAV-5, AAV-6, etc. or hybrid AAV vectors comprising the same) is an example of a vector for use in the present methods for delivering one or more oligonucleotides complementary to a sense or antisense chimeric ncmtRNA molecule to cancer cells (such as a plasmocyte; see, e.g. U.S. Patent Application Publication No. 2004/0224389, the disclosure of which is incorporated by reference herein, or a CSC). In some embodiments, recombinant vector (e.g., a viral vector) described herein can be used in any method described herein. In some embodiments, a method of suppressing metastasis of a cancer in an individual comprises administering to the individual an effective amount of a recombinant vector (e.g., a viral vector) comprising one or more oligonucleotide described herein. In some embodiments, a method for treating or preventing relapse of cancer in an individual comprises administering to the individual an effective amount of a recombinant vector (e.g., a viral vector) comprising one or more oligonucleotide described herein. In some embodiments, a method for treating metastatic cancer in an individual comprises administering to the individual an effective amount of a recombinant vector (e.g., a viral vector) comprising one or more oligonucleotide described herein. In some embodiments, a method for treating a refractory cancer (e.g., a refractory HPV-associated cancer) in an individual comprises administering to the individual an effective amount of a recombinant vector (e.g., a viral vector) comprising one or more oligonucleotide described herein. In some embodiments, the individual has been previously treated for cancer with a therapy (e.g., chemotherapy, radiation therapy, surgery or combinations thereof).

In another aspect, one or more oligonucleotides (such as any of the oligonucleotides disclosed herein) complementary to a sense and/or antisense chimeric non-coding mitochondrial RNA molecule is encapsulated within a microcarrier for deliver to an individual. In certain embodiments, a mixture of different oligonucleotides (such as any of the oligonucleotides disclosed herein) complementary to a sense and/or antisense chimeric non-coding mitochondria RNA molecule may be encapsulated with a microcarrier, such that the microcarrier encapsulates more than one oligonucleotide species. In some embodiments, the one or more oligonucleotides (such as any of the oligonucleotides disclosed herein) complementary to a sense and/or antisense chimeric non-coding mitochondrial RNA molecule encapsulated within the microcarrier comprises a nucleic acid sequence selected from the group consisting of SEQ ID NOs:7-198. In some embodiments, the one or more oligonucleotides (such as any of the oligonucleotides disclosed herein) complementary to a sense and/or antisense chimeric non-coding mitochondrial RNA molecule encapsulated within the microcarrier comprises a nucleic acid sequence selected from the group consisting of SEQ ID NOs:36, 197 and 198.

Methods of encapsulating oligonucleotides in microcarriers are well known in the art, and described, for example, International application WO098/55495. Colloidal dispersion systems, such as microspheres, beads, macromolecular complexes, nanocapsules and lipid-based system, such as oil-in-water emulsions, micelles, mixed micelles and liposomes can provide effective encapsulation of oligonocelotides within microcarrier compositions. The encapsulation composition may further comprise any of a wide variety of components. These include, but are not limited to, alum, lipids, phospholipids, lipid membrane structures (LMS), polyethylene glycol (PEG) and other polymers, such as polypeptides, glycopeptides, and polysaccharides.

Other Anti-Cancer Therapies

In some aspects, any of the methods of treatment described herein can comprise administering one or more additional anti-cancer therapies to the individual. In some embodiments, the one or more anti-cancer therapy is selected from the group consisting of chemotherapy, radiation therapy, and surgery. Chemotherapy and anti-cancer agents are used interchangeably herein. Various classes of anti-cancer agents can be used. Non-limiting examples include: alkylating agents, antimetabolites, anthracyclines, plant alkaloids, topoisomerase inhibitors, podophyllotoxin, antibodies (e.g., monoclonal or polyclonal), tyrosine kinase inhibitors (e.g., imatinib mesylate (Gleevec® or (Glivec®)), hormone treatments, soluble receptors and other antineoplastics.

Topoisomerase inhibitors are also another class of anti-cancer agents that can be used herein. Topoisomerases are essential enzymes that maintain the topology of DNA. Inhibition of type I or type II topoisomerases interferes with both transcription and replication of DNA by upsetting proper DNA supercoiling. Some type I topoisomerase inhibitors include camptothecins: irinotecan and topotecan. Examples of type II inhibitors include amsacrine, etoposide, etoposide phosphate, and teniposide. These are semisynthetic derivatives of epipodophyllotoxins, alkaloids naturally occurring in the root of American Mayapple (Podophyllum peltatum).

Antineoplastics include the immunosuppressant dactinomycin, doxorubicin, epirubicin, bleomycin, mechlorethamine, cyclophosphamide, chlorambucil, ifosfamide. The antineoplastic compounds generally work by chemically modifying a cell's DNA.

Alkylating agents can alkylate many nucleophilic functional groups under conditions present in cells. Cisplatin and carboplatin, and oxalipiatin are alkylating agents. They impair cell function by forming covalent bonds with the amino, carboxyl, sulfhydryl, and phosphate groups in biologically important molecules.

Vinca alkaloids bind to specific sites on tubulin, inhibiting the assembly of tubulin into microtubules (M phase of the cell cycle). The vinca alkaloids include: vincristine, vinblastine, vinorelbine, and vindesine.

Anti-metabolites resemble purines (azathioprine, mercaptopurine) or pyrimidine and prevent these substances from becoming incorporated in to DNA during the “S” phase of the cell cycle, stopping normal development and division. Anti-metabolites also affect RNA synthesis.

Plant alkaloids and terpenoids are derived from plants and block cell division by preventing microtubule function. Since microtubules are vital for cell division, without them, cell division cannot occur. The main examples are vinca alkaloids and taxanes.

Podophyllotoxin is a plant-derived compound which has been reported to help with digestion as well as used to produce two other cytostatic drugs, etoposide and teniposide. They prevent the cell from entering the G1 phase (the start of DNA replication) and the replication of DNA (the S phase).

Taxanes as a group includes paclitaxel and docetaxel. Paclitaxel is a natural product, originally known as Taxol and first derived from the bark of the Pacific Yew tree. Docetaxel is a semi-synthetic analogue of paclitaxel. Taxanes enhance stability of microtubules, preventing the separation of chromosomes during anaphase.

In some aspects, the anti-cancer agent can be selected from remicade, docetaxel, celecoxib, melphalan, dexamethasone (Decadron®), steroids, gemcitabine, cisplatinum, temozolomide, etoposide, cyclophosphamide, temodar, carboplatin, procarbazine, gliadel, tamoxifen, topotecan, methotrexate, gefitinib (Iressa®), taxol, taxotere, fluorouracil, leucovorin, irinotecan, xeloda, CPT-11, interferon alpha, pegylated interferon alpha (e.g., PEG INTRON-A), capecitabine, cisplatin, thiotepa, fludarabine, carboplatin, liposomal daunorubicin, cytarabine, doxetaxol, vinblastine, IL-2, GM-CSF, dacarbazine, vinorelbine, zoledronic acid, palmitronate, biaxin, busulphan, prednisone, bortezomib (Velcade®), bisphosphonate, arsenic trioxide, vincristine, doxorubicin (Doxil®), paclitaxel, ganciclovir, adriamycin, estrainustine sodium phosphate (Emcyt®), sulindac, or etoposide.

In other embodiments, the anti-cancer agent can be selected from bortezomib, cyclophosphamide, dexamethasone, doxorubicin, interferon-alpha, lenalidomide, melphalan, pegylated interferon-alpha, prednisone, thalidomide, or vincristine.

In some aspects, the one or inure anti-cancer therapy is radiation therapy. As used herein, the term “radiation therapy” refers to the administration of radiation to kill cancerous cells. Radiation interacts with molecules in the cell such as DNA to induce cell death. Radiation can also damage the cellular and nuclear membranes and other organelles. Depending on the radiation type, the mechanism of DNA damage may vary as does the relative biologic effectiveness. For example, heavy particles (i.e. protons, neutrons) damage DNA directly and have a greater relative biologic effectiveness. Electromagnetic radiation results in indirect ionization acting through short-lived, hydroxyl free radicals produced primarily by the ionization of cellular water. Clinical applications of radiation consist of external beam radiation (from an outside source) and brachytherapy (using a source of radiation implanted or inserted into the patient). External beam radiation consists of X-rays and/or gamma rays, while brachytherapy employs radioactive nuclei that decay and emit alpha particles, or beta particles along with a gamma ray. Radiation also contemplated herein includes, for example, the directed delivery of radioisotopes to cancer cells. Other forms of DNA damaging factors are also contemplated herein such as microwaves and UV irradiation.

Radiation may be given in a single dose or in a series of small doses in a dose-fractionated schedule. The amount of radiation contemplated herein ranges from about 1 to about 100 Gy, including, for example, about 5 to about 80, about 10 to about 50 Gy, or about 10 Gy. The total dose may be applied in a tractioned regime. For example, the regime may comprise fractionated individual doses of 2 Gy. Dosage ranges for radioisotopes vary widely, and depends on the half-life of the isotope and the strength and type of radiation emitted. When the radiation comprises use of radioactive isotopes, the isotope may be conjugated to a targeting agent, such as a therapeutic antibody, which carries the radionucleotide to the target tissue (e.g., tumor tissue). Suitable radioactive isotopes include, but are not limited to, astatine²¹¹, ¹⁴carbon, ⁵¹chromium, ³⁶chlorine, ^{s7}iron, ⁵⁸cobalt, copper⁶⁷, ¹⁵²Eu, gallium⁶⁷, \hat{ }hydrogen, iodine¹²³, iodine¹³¹, indium¹¹¹, ⁵⁹ion, ³²phosphorus, rhenium¹⁸⁶, ⁷⁵selenium, ³⁵sulphur, technicium^{99m}, and/or yttrium°.

Surgery described herein includes resection in which all or part of a cancerous tissue is physically removed, exercised, and/ or destroyed. Tumor resection refers to physical removal of at least part of a tumor. In addition to tumor resection, treatment by surgery includes laser surgery, cryosurgery, electrosurgery, and micropically controlled surgery (Mohs surgery). Removal of precancers or normal tissues is also contemplated herein.

Stem Cell Transplantation and Ex Vivo Treatment of Autologous Hematopoietic Stem Cells

In other aspects, any of the methods of treatment described herein can include either autologous or allogenic stem cell transplantation therapy. In recent years, high-dose chemotherapy with autologous hematopoietic stem-cell transplantation has become the preferred treatment for certain cancers such as multiple myeloma, non-Hodgkin lymphoma, Hodgkin lymphoma, and leukemia. While not curative, this procedure does prolong overall survival and complete remission. Prior to stem-cell transplantation, patients receive an initial course of induction chemotherapy. The most common induction regimens used today are thalidomide-dexamethasone, bortezomib based regimens, and lenalidomide-dexamethasone (Kyle & Rajkumar, Blood, 111 (6): 2962-72, 2008). For example, autologous peripheral stem cell transplantation is useful for up to 50% of multiple myeloma patients. Despite a low mortality rate, problems with such transplant therapy include the inability to eradicate the tumor and the difficulty in the removal of myeloma cells and their precursors from the stem cell collection used for transplantation.

Allogenic transplant (the transplantation of a healthy person's stem cells into the affected individual), is another therapy option for treating certain cancers such as multiple myeloma, non-Hodgkin lymphoma, Hodgkin lymphoma, and leukemia but is less frequently used as it may not provide a cure. For example, most studies evaluating its use in multiple myeloma patients demonstrate long-term disease-free survival of 10-20%, with a significant fraction of patients developing relapse.

When included as a treatment for suppressing or preventing metastasis according to any of the methods disclosed herein, autologous stem cell transplantation can also include the step of treating the hematopoietic stem-cells and/or bone marrow to be transplanted into the affected individual with any of the anti-cancer agents disclosed herein, prior to transplantation into the affected individual. In one embodiment, hematopoietic stem-cells and/or bone marrow for use in autologous stein cell transplantation can be treated with an effective amount of one or more oligonucleotides antisense oligonucleotides) sufficiently complementary to an ASncmtRNA or SncmtRNA molecule (e.g., any of the ASncmtRNA and/or SncmtRNA molecules disclosed herein) to form a stable duplex prior to transplantation into the affected individual. In another embodiment, the one or more oligonucleotide is sufficiently complementary to one or more ncmtRNA encoded by a nucleic acid sequence selected from the group consisting of SEQ ID NOs:1-6, to form a stable duplex. In other embodiments, the one or more oligonucleotide comprises a nucleic acid sequence selected from the group consisting of SEQ ID NOs:7-198. In some embodiments, the one or more oligonucleotide comprises a nucleic acid sequence selected from the group consisting of SEQ ID NOs:36, 197 and 198.

It has been shown that autologous transplantation of bone marrow or hematological stem cells can also be used to treat several forms of hematological cancers (such as, but not limited to, multiple myeloma, leukemia and lymphoma). Accordingly, in some aspects, when included as a treatment for a hematological cancer, provided herein is a method of performing autologous stern cell transplantation which includes the step of treating the hematopoietic stem-cells and/or bone marrow to be transplanted into the affected individual with any of the anti-cancer agent disclosed herein, prior to transplantation into the affected individual. In one embodiment, hematopoietic stem-cells and/or bone marrow for use in autologous stern cell transplantation in an individual with a hematological cancer can be treated with an effective amount of one or more oligonucleotides antisense oligonucleotides) sufficiently complementary to an ASncmtRNA molecule or SncmtRNA molecule (e.g., any of the ASncmtRNA and/or SncmtRNA molecules disclosed herein) to form a stable duplex prior to transplantation into the affected individual. In another embodiment, the one or more oligonucleotide is sufficiently complementary to one or more ncmtRNA encoded by a nucleic acid sequence selected from the group consisting of SEQ ID NOs:1-6, to form a stable duplex. In other embodiments, the one or more oligonucleotide comprises a nucleic acid sequence selected from the group consisting of SEQ ID NOs:7-198. In some embodiments, the one or more oligonucleotide comprises a nucleic acid sequence selected from the group consisting of SEQ ID NOs:36, 197 and 198.

### IV. Compositions

Any of the anti-cancer agents (such as oligonucleotide-based agents) disclosed herein can be administered in the form of compositions (e.g., pharmaceutical compositions). These compounds can be administered by systemic administration or local administration through various routes. The route(s) of administration useful in a particular application are apparent to one of skill in the art. Routes of administration include but are not limited to oral, rectal, cerebrospinal, transdermal, subcutaneous, topical, transmucosal, nasopharangeal, pulmonary, intravenous, intramuscular, and intranasal. In some embodiments, the administration is a local administration. In some embodiments, the local administration is selected from the group consisting of administration into an organ, into a cavity, into a tissue, and subcutaneous administration. In some embodiments, the administration is systemic administration. In some embodiments, the systemic administration is intravenous or intraperitoneal administration. These compounds are effective as both injectable and oral compositions. Such compositions are prepared in a manner well known in the pharmaceutical art and comprise at least one active compound. The compositions herein may also contain more than once active compound as necessary for the particular indication being treated, preferably those with complementary activities that do not adversely affect each other. When employed as oral compositions, the oligonucleotides, and other anti-cancer agents disclosed herein, are protected from acid digestion in the stomach by a pharmaceutically acceptable protectant.

This invention also includes pharmaceutical compositions which contain, as the active ingredient, one or more of the anti-cancer agents disclosed herein associated with one or more pharmaceutically acceptable excipients or carriers. In making the compositions of this invention, the active ingredient is usually mixed with an excipient or carrier, diluted by an excipient or carrier or enclosed within such an excipient or carrier which can be in the form of a capsule, sachet, paper or other container. When the excipient or carrier serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In some embodiments, in preparing a formulation, it may be necessary to mill the active lyophilized compound to provide the appropriate particle size prior to combining with the other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, e.g. about 40 mesh.

Some examples of suitable excipients or carriers include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The compositions can be formulated in a unit dosage form, each dosage containing from about 5 mg to about 100 mg or more, such as any one of about 1 mg to about 5 mg, 1 mg to about 10 mg, about 1 mg to about 20 mg, about 1 mg to about 30 mg, about 1 mg to about 40 mg, about 1 mg to about 50 mg, about 1 mg to about 60 mg, about 1 mg to about 70 mg, about 1 mg to about 80 mg, or about 1 mg to about 90 mg, inclusive, including any range in between these values, of the active ingredient. The term “unit dosage forms” refers to physically discrete units suitable as unitary dosages for individuals, each unit containing a predetermined quantity of active material calculated to produce the desired therapeutic effect, in association with a suitable pharmaceutical excipient or carrier.

The anti-cancer agents (such as oligonucleotide-based agents) disclosed herein are effective over a wide dosage range and are generally administered in a therapeutically effective amount. It will be understood, however, that the amount of the anti-cancer agents actually administered will be determined by a physician, in the light of the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient anticancer therapy is mixed with a pharmaceutical excipient or carrier to form a solid preformulation composition containing a homogeneous mixture of a compound of the present invention. When referring to these preformulation compositions as homogeneous, it is meant that the active ingredient is dispersed evenly throughout the composition so that the composition can be readily subdivided into equally effective unit dosage forms such as tablets, pills and capsules.

The tablets or pills of the present invention can be coated or otherwise compounded to provide a dosage form affording the advantage of prolonged action and to protect the anticancer therapies (such as an oligonucleotide) from acid hydrolysis in the stomach. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compositions of the present invention can be incorporated for administration orally or by injection include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles.

Parenteral routes of administration include but are not limited to direct injection into a central venous line, intravenous, intramuscular, intraperitoneal, intradermal, or subcutaneous injection. Oligonucleotide (e.g., an oliconucleotide and microcarrier formulation) formulations suitable for parenteral administration are generally formulated in USP water or water for injection and may further comprise pH buffers, salts bulking agents, preservatives, and other pharmaceutically acceptable excipients. Oligonucleotide(s), for example as oligonucleotide microcarrier complexes or encapsulates, for parenteral injection may be formulated in pharmaceutically acceptable sterile isotonic solutions such as saline and phosphate buffered saline for injection.

Compositions for inhalation or insufflation include solutions and suspensions in pharmaceutically acceptable, aqueous or organic solvents, or mixtures thereof, and powders. The liquid or solid compositions can contain suitable pharmaceutically acceptable excipients as described herein. The compositions can be administered by the oral or nasal respiratory route for local or systemic effect. Compositions in pharmaceutically acceptable solvents can be nebulized by use of inert gases. Nebulized solutions can be inhaled directly from the nebulizing device or the nebulizing device can be attached to a face mask tent, or intermittent positive pressure breathing machine. Solution, suspension, or powder compositions can also be administered, orally or nasally, from devices which deliver the formulation in an appropriate manner.

### Methods of Treatment

A. Methods for Suppressing or Preventing Metastasis of a Cancer

In one aspect, provided herein is one or more oligonucleotide (or composition thereof) for use in suppressing or preventing metastasis of a cancer in an individual. In another aspect, provided herein is one or more oligonucleotide (or compositions thereof) for use in combination with at least one therapy for suppressing or preventing metastasis of a cancer in an individual. In any of the aspects, herein, the individual may have been previously treated for cancer with a therapy.

In some aspects, the invention provides a method for suppressing metastasis of a cancer in an individual comprising administering to the individual an effective amount of one or more oligonucleotide described herein, wherein the oligonucleotide is able to hybridize with the chimeric mitochondrial RNA molecules to form a stable duplex, and wherein the individual has been previously treated for cancer with a therapy. In a further embodiment, the method for suppressing metastasis of a cancer in an individual comprises administering the one or more oligonucleotide in combination with at least one therapy disclosed herein. In some embodiments, the at least one therapy is selected from the group consisting of an anti-cancer agent, a radiation therapy, surgery, an allogenic stein cell transplant therapy, and an autologous stem cell transplant therapy. In some of the embodiments herein, the individual has been previously treated for cancer with a therapy comprising chemotherapy, radiation therapy, surgery, or combinations thereof. In some embodiments, the individual has been previously treated with one or more of bortezomib, cyclophosphamide, dexamethasone, doxorubicin, interferon-alpha, lenalidomide, melphalan, pegylated interferon-alpha, prednisone, thalidomide, and vincristine. In any of the embodiments herein, the oligonucleotide and the at least one therapy is administered sequentially. For example, one or more oligonucleotide described herein can be administered to an individual before or after a tumor(s) has been surgically resected from the individual. In some embodiments, the oligonucleotide and the at least one therapy is administered simulatenously. For example, one or more oligonucleotide described herein can be administered to an individual during surgical resection of a tumor(s) from the individual.

In some aspects, the invention provides a method for preventing metastasis of a cancer in an individual comprising administering to the individual an effective amount of one or more oligonucleotide described herein, wherein the oligonucleotide is able to hybridize with the chimeric mitochondrial RNA molecules to form a stable duplex, and wherein the individual has been previously treated for cancer with a therapy. In a further embodiment, the method for suppressing or preventing metastasis of a cancer in an individual comprises administering the one or more oligonucleotide in combination with at least one therapy disclosed herein. In sonic embodiments, the at least one therapy is selected from the group consisting of an anti-cancer agent, a radiation therapy, surgery, an allogenic stem cell transplant therapy, and an autologous stem cell transplant therapy. In some of the embodiments herein, the individual has been previously treated for cancer with a therapy comprising chemotherapy, radiation therapy, surgery, or combinations thereof. In some embodiments, the individual has been previously treated with one or more of bortezomib, cyclophosphamide, dexamethasone, doxorubicin, interferon-alpha, lenalidomide, melphalan, pegylated interferon-alpha, prednisone, thalidomide, and vincristine. In any of the embodiments herein, the oligonucleotide and the at least one therapy is administered sequentially. For example, one or more oligonucleotide described herein can be administered to an individual before or after a tumor(s) has been surgically resected from the individual. In some embodiments, the oligonucleotide and the at least one therapy is administered simulatenously. For example, one or more oligonucleotide described herein can be administered to an individual during surgical resection of a tumor(s) from the individual.

As non-limiting examples, a method for suppressing or preventing metastasis of cancer according to the present invention may be by administration of one or more oligonucleotide (or a composition thereof) described herein provided as a daily dosage in an amount of about 0.1 to about 100 mg/kg, such as about 0.5, about 0.9, about 1.0, about 1.1, about 1.5, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 40, about 45, about 50, about 60, about 70, about 80, about 90 or about 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25.26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18,19 or 20 after initiation of treatment, or any combination thereof, using single or divided doses at every 24, 12, 8, 6, 4, or 2 hours, or any combination thereof.

In some embodiments, the one or more oligonucleotide (or a composition thereof) may be administered in combination with at least one therapy (e.g., an anti-cancer agent, a radiation therapy, surgery, an allogenic stem cell transplant therapy, or an autologous stem cell transplant therapy). In some embodiments, the combination is administered sequentially. For example, a one or more oligonucleotide described herein may be administered about 1, 2, 3, 4, 5, 6, 7, 8, 9, 10, 11, 12, 13, 14, 15, 16, 17, 18, 19, 20, 21 22, 23, 24, 25, 26, 27, 28, 29 30, 31, 32, 33, 34, 35, 36, 37, 38, 39, or 40 days, or alternatively, about 1, 2, 3, 4, 5, 6, 7, 8, 9, 10, 11, 12, 13, 14, 15, 16, 17, 18, 19 or 20 weeks apart from the asministration of the at least one therapy during combination treatment. In some embodiments, the combination is administered simultaneously. For example, a one or more oligonucleotide described herein may be administered about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, or 59 minutes, or alternatively, about 1, 2, 3, 4, 5, 6, 7, 8, 9, 10, 11, 12, 13, 14, 15, 16, 17, 18, 19, 20, 21, 22, 23, or 24 hours apart from the administration of the at least one therapy during combination treatment.

B. Methods for Treating or Preventing Relapse of a Cancer

In other aspects, provided herein is one or more oligonucleotide (or compositions thereof) for use in treating or preventing relapse of a cancer in an individual. In some embodiments, the individual has responded to initial treatment and is in remission.

In some aspects, the invention provides a method for treating or preventing relapse of cancer in an individual comprising administering to the individual an effective amount of one or more oligonucleotide described herein, wherein the oligonucleotide is able to hybridize with the chimeric mitochondrial RNA molecules to form a stable duplex, and wherein the individual has been previously treated for cancer with a therapy. In a further embodiment, the method for treating or preventing relapse of cancer in an individual comprises administering the one or more oligonucleotide in combination with at least one therapy disclosed herein. In some embodiments, the at least one therapy is selected from the group consisting of an anti-cancer agent, a radiation therapy, surgery, an allogenic stem cell transplant therapy, and an autologous stem cell transplant therapy. In some of the embodiments herein, the individual has been previously treated for cancer with a therapy comprising chemotherapy, radiation therapy, surgery, or combinations thereof. In some embodiments, the individual has been previously treated with one or more of bortezomib, cyclophosphamide, dexamethasone, doxorubicin, interferon-alpha, lenalidomide, melphalan, pegylated interferon-alpha, prednisone, thalidomide, and vincristine. In any of the embodiments herein, the oligonucleotide and the at least one therapy is administered sequentially. For example, one or more oligonucleotide described herein can be administered to an individual before or after a tumor(s) has been surgically resected from the individual In some embodiments, the oligonucleotide and the at least one therapy is administered simulatenously. For example, one or more oligonucleotide described herein can be administered to an individual during surgical resection of a tumor(s) from the individual.

As non-limiting examples, a method for treating or preventing relapse of a cancer according to the present invention may be by administration of one or more oligonucleotide (or a composition thereof) described herein provided as a daily dosage in an amount of about 0.1 to about 100 mg/kg, such as about 0.5, about 0.9, about 1.0, about 1.1, about 1.5, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 40, about 45, about 50, about 60, about 70, about 80, about 90 or about 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19 or 20 after initiation of treatment, or any combination thereof, using single or divided doses at every 24, 12, 8, 6, 4, or 2 hours, or any combination thereof.

In some embodiments, the one or more oligonucleotide (or a composition thereof) may be administered in combination with at least one therapy (e.g., an anti-cancer agent, a radiation therapy, surgery, an allogenic stem cell transplant therapy, or an autologous stem cell transplant therapy). In some embodiments, the combination is administered sequentially. For example, a one or more oligonucleotide described herein may be administered about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days, or alternatively, about 1, 2, 3, 4, 5, 6, 7, 8, 9, 10, 11, 12, 13, 14, 15, 16, 17, 18, 19 or 20 weeks apart from the asministration of the at least one therapy during combination treatment. In some embodiments, the combination is administered simultaneously. For example, a one or more oligonucleotide described herein may be administered about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, or 59 minutes, or alternatively, about 1, 2, 3, 4, 5, 6, 7, 8, 9, 10, 11, 12, 13, 14, 15, 16, 17, 18, 19, 20, 21, 22, 23, or 24 hours apart from the administration of the at least one therapy during combination treatment.

In other embodiments, a “maintenance schedule” may be used in which one or more maintenance oligonucleotide-based (such as antisense-based) therapies are administered less frequency than in the original treatment administered prior to remission, such as once per week or once every two weeks. The maintenance schedule can be continued either for a fixed period of time, generally about 1 or about 2 years, or indefinitely as long as the patient is continuing to show no signs of progressive disease and is tolerating the treatment without significant toxicity.

C. Methods for Treating Metastatic Cancer

In yet other aspects, provided herein is one or more oligonucleotide (or compositions thereof) for use in treating metastatic cancer (such as relapsed metastatic cancer) in an individual.

In some aspects, the invention provides a method for the treatment of metastatic cancer (such as relapsed metastatic cancer) in an individual comprising administering to the individual an effective amount of one or more oligonucleotide described herein, wherein the oligonucleotide is able to hybridize with the chimeric mitochondrial RNA molecules to form a stable duplex, and wherein the individual has been previously treated for cancer with a therapy. In a further embodiment, the method for treating metastatic cancer (such as relapsed metastatic cancer) in an individual comprises administering the one or more oligonucleotide in combination with at least one therapy disclosed herein. In some embodiments, the at least one therapy is selected from the group consisting of an anti-cancer agent, a radiation therapy, surgery, an allogenic stem cell transplant therapy, and an autologous stem cell transplant therapy. In some of the embodiments herein, the individual has been previously treated for cancer with a therapy comprising chemotherapy, radiation therapy, surgery, or combinations thereof. In any of the embodiments herein, the oligonucleotide and the at least one therapy is administered sequentially. For example, one or more oligonucleotide described herein can be administered to an individual before or after a tumor(s) has been surgically resected from the individual. In some embodiments, the oligonucleotide and the at least one therapy is administered simulatenously. For example, one or more oligonucleotide described herein can be administered to an individual during surgical resection of a tumor(s) from the individual.

As non-limiting examples, a method for the treatment of metastatic cancer (such as relapsed metastatic cancer) according to the present invention may be by administration of one or more oligonucleotide (or a composition thereof) described herein provided as a daily dosage in an amount of about 0.1 to about 100 mg/kg, such as about 0.5, about 0.9, about 1.0, about 1.1, about 1.5, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 40, about 45, about 50, about 60, about 70, about 80, about 90 or about 100 mg/kg, per day, on at least one of day 1, 2, 3, 4, 5, 6, 7, 8, 9, 10, 11, 12, 13, 14, 15, 16, 17, 18, 19, 20, 21 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19 or 20 after initiation of treatment, or any combination thereof, using single or divided doses at every 24, 12, 8, 6, 4, or 2 hours, or any combination thereof.

In some embodiments, the one or more oligonucleotide (or a composition thereof) may be administered in combination with at least one therapy (e.g., an anti-cancer agent, a radiation therapy, surgery, an allogenic stem cell transplant therapy, or an autologous stem cell transplant therapy). In some embodiments, the combination is administered sequentially. For example, a one or more oligonucleotide described herein may be administered about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days, or alternatively, about 1, 2, 3, 4, 5, 6, 7, 8, 9, 10, 11, 12, 13, 14, 15, 16, 17, 18, 19 or 20 weeks apart from the asministration of the at least one therapy during combination treatment. In some embodiments, the combination is administered simultaneously. For example, a one or more oligonucleotide described herein may be administered about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, or 59 minutes, or alternatively, about 1, 2, 3, 4, 5, 6, 7, 8, 9, 10, 11, 12, 13, 14, 15, 16, 17, 18, 19, 20, 21, 22, 23, or 24 hours apart from the administration of the at least one therapy during combination treatment.

In another embodiment, the therapeutically effective amount of said one or more oligonucleotide (or compositions thereof) is administered as part of a salvage therapy in treating an individual wherein the cancer has become refractory to other treatment for cancer. In some embodiments, the individual relapsed after treatment with one or more of bortezomib, cyclophosphamide, dexamethasone, doxorubicin, interferon-alpha, lenalidomide, melphalan, pegylated interferon-alpha, prednisone, thalidomide, and vincristine.

Without being bound by theory, it is believed that CSCs give rise to the relapse and/or metastasis of a cancer following treatment of the primary tumor. In some aspects, methods of treatment as described herein (e.g., a method for suppressing or preventing metastasis of a cancer, etc.) eliminates or supresses cancer stem cells. In some embodiments, the oligonucleotides disclosed herein can kill or inhibit cancer stem cells (CSCs) to suppress metastasis, prevent metastasis or prevent relapse of a cancer in an individual. In some embodiments, the individual has been previously treated for cancer with a therapy. In one embodiment, the oligonucleotides disclosed herein can kill or inhibit cancer stem cells (CSCs) that are resistant to treament (e.g., chemotherapy). In one embodiment, treatment of an individual with any one or more oligonucleotide disclosed herein (e.g., an oligonucleotide complementary to an ASncmtRNA molecule) non-selectively inhibits, arrests, kills, or abolishes the CSCs in the individual. In one embodiment, any one or more oligonucleotide disclosed herein (e.g., an oligonucleotide complementary to an ASncmtRNA molecule) reduces the number of CSCs in the individual as compared to an individual not administered the oligonucleotide. In a further embodiment, the individual has been previously treated for cancer with a therapy.

In any embodiments of the methods herein, any one or more oligonucleotide disclosed herein (e.g., an oligonucleotide complementary to an ASncmtRNA molecule) inhibits tumor growth and/or metastasis in the individual as compared to an individual not administered the oligonucleotide.

In any of the embodiments of the methods herein (e.g., a method for suppressing or preventing metastasis of a cancer, a method for treating or preventing relapse of a cancer, a method for treating metaststic cancer, etc.), the cancer may be a solid cancer or a non-solid cancer. In any of the embodiments herein, the cancer is a solid cancer. Examples of solid cancers contemplated herein include, without limitation, squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, brain cancer, cervical cancer, ovarian cancer, liver cancer, sarcoma, bladder cancer, hepatoma, breast cancer, colon cancer, colorectal cancer, endometrial or uterine carcinoma, oralpharyngeal cancer, salivary gland carcinoma, renal cancer, liver cancer, prostate cancer, vulval cancer, thyroid cancer, hepatic carcinoma, gastric cancer, melanoma, and various types of head and neck cancer.

In some embodiments, the cancer is associated with a human papilloma virus (HPV) infection, also referred to herein as “HPV-associated cancer”, such as in cervical cancer, oralpharyngeal cancer, and head and neck cancer. For example, one of the most important risk factors for development of cervical cancer is an HPV infection. Over 100 strains of HPV have been identified, however, only a subset are classified as high-risk (16, 18, 31, 33, 35, 39, 45, 51, 52, 56, 58, 59, 68, 73, and 82) or probable high-risk (26, 53, and 66) types for the development of cancer (Munoz et al., NEJM, 348:518-527, 2003). Of these HPV types, HPV16 and HPV18 are reported to cause nearly 70% of all cervical cancer cases while HPV 31 and 35 cause another 10% of cervical cancer cases. See Walboomers et al., J Pathol., 189(1):12-9, 1999. HPV-associated cancer can involve one or more of the following steps: (1) initial HPV infection, (2) persistent HPV infection, (3) transforming HPV infection, in the presence or absence of integration of HPV DNA into the host cell genome, (4) development of precancerous lesions, (5) development of at least one primary tumor, and (6) development of invasive cancer (e.g., metastatic cancer).

In some instances, the HPV-associated cancer is resistant to chemotherapeutic agents regularly used for the treatment of cancer. For example, HPV 16-immortalized cervical cells can develop resistance to cisplatin, paclitaxel, actinomycin D, doxrubucin, etoposide, and 5-fluorouracil which presents a major obstacle in cancer treatment. See Ding et al., Int J Cancer, 15:87(6)818-23, 2000. In some embodiments herein, provided herein is one or more oligonucleotide (or compositions thereof) for use in suppressing metastasis of a cancer in an individual, wherein the cancer is resistant to a chemotherapeutic agent, and wherein the cancer is an HPV-associated cancer. In some embodiments herein, provided herein is one or more oligonucleotide (or compositions thereof) for use in treating or preventing relapse of a cancer in an individual, wherein the cancer is resistant to a chemotherapeutic agent, and wherein the cancer is an HPV-associated cancer. In some embodiments herein, provided herein is one or more oligonucleotide (or compositions thereof) for use in treating metastatic cancer (such as relapsed metastatic cancer) in an individual, wherein the metastatic cancer is resistant to a chemotherapeutic agent, and wherein the metastatic cancer is an HPV-associated cancer. In some embodiments herein, provided herein is one or more oligonucleotide (or compositions thereof) for use in treating a refractory cancer in an individual. In some embodiments, the refractory cancer is a refractory HPV-associated cancer. In some embodiments, the refractory HPV-associated cancer is resistant to a chemotherapeutic agent. As used herein, the term “refractory cancer” refers to a cancer (e.g., an HPV-associated cancer) that does not respond to treatment, for example, a cancer that is resistant at the beginning of treatment (e.g., treatment with a chemotherapeutic agent) or a cancer that may become resistant during treatment. In some embodiments, the chemotherapeutic agent is selected from the group consisting of cisplatin, paclitaxel, actinomycin D, doxrubucin, etoposide, and 5-fluorouracil. In some embodiments, the chemotherapeutic agent is cisplatin. In some of the embodiments herein, the HPV-associated cancer (e.g., a refractory HPV-associated cancer) is from an infection with one or more HPV strains selected from the group consisting of HPV 16, FIPV 18, HPV 31 and HPV 45. In some of the embodiments herein, the HPV-associated cancer (e.g., a refractory HPV-associated cancer) is from an infection with the HPV strain HPV 45. In some embodiments, the one or more oligonucleotide is complementary to the SncmtRNA molecule encoded by a nucleotide sequence selected from the group consisting of SEQ ID NO:1, SEQ ID NO:2, and SEQ ID NO:3. In some embodiments, the one or more oligonucleotide is complementary to the ASncmtRNA molecule encoded by a nucleotide sequence selected from the group consisting of SEQ ID NO:4, SEQ ID NO:5, and SEQ ID NO:6. In some embodiments, the one or more oligonucleotide comprises a nucleotide sequence selected from the group consisting of SEQ NOs:7-198. In some embodiments, the one or more oligonucleotide comprises a nucleic acid sequence selected from the group consisting of SEQ ID NOs:36, 197 and 198.

In some embodiments, provided herein is one or more oligonucleotide (or compositions thereof) for use in treating a refractory cancer in an individual. In some embodiments, the refractory cancer is resistant to a chemotherapeutic agent. In some embodiments, the chemotherapeutic agent is cisplatin. In some embodiments, the refractory cancer is a solid cancer disclosed herein. For example, the refractory solid cancer may be one or more of bladder cancer, brain cancer, breast cancer, cervical cancer (e.g., a refractory HPV-associated cervical cancer), colon cancer, endometrial cancer, esophageal cancer, gastric cancer, liver and bile duct cancer, lung cancer, melanoma, oral cancer, ovarian cancer, pancreatic cancer, pharynx cancer, prostate cancer, renal cancer, testicular cancer, or thyroid cancer. In some embodiments, the refractory cancer is a non-solid cancer disclosed herein. For example, the refractory cancer may be one or more of multiple myeloma, leukemia, or lymphoma.

In any of the embodiments herein, the cancer is a non-solid cancer. “Non-solid cancer” refers to a hematological malignancy involving abnormal growth and/or metastasis of a blood cell. Examples of non-solid cancers contemplated herein include, without limitation, multiple myeloma, acute myeloid leukemia, acute lymphoblastic leukemia, chronic myelogenous leukemia, chronic lymphocytic leukemia, acute nonlymphocytic leukemia, acute granulocytic leukemia, chronic granulocytic leukemia, acute promyelocytic leukemia, adult T-cell leukemia, aleukemic leukemia, a leukocythemic leukemia, basoplaylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, undifferentiated cell leukemia, idiopathic myelofibrosis, lymphoma (such as Non-Hodgkin's lymphoma, and Hodgkin's lymphoma), and myelodysplastic syndrome.

The methods disclosed herein can be practiced in an adjuvant setting. “Adjuvant setting” can refers to a clinical setting in which an individual has had a history of cancer, and generally (but not necessarily) been responsive to therapy, which includes, but is not limited to, surgery (such as surgical resection), radiotherapy, and chemotherapy. However, because of their history of the cancer (such as melanoma or colon cancer), these individuals are considered at risk of development of cancer. Treatment or administration in the “adjuvant setting” refers to a subsequent mode of treatment. The degree of risk (i.e., when an individual in the adjuvant setting is considered as “high risk” or “low risk”) depends upon several factors, most usually the extent of disease (cancer) when first treated.

The present invention is accordingly directed to methods for inhibiting the symptoms or conditions (disabilities, impairments) associated with cancer (e.g., metastatic cancer or relapsed cancer) as described in detail below. As such, it is not required that all effects of the condition be entirely prevented or reversed, although the effects of the presently disclosed methods likely extend to a significant therapeutic benefit for the individual. As such, a therapeutic benefit is not necessarily a complete prevention or cure for the condition, but rather, can encompass a result which includes reducing or preventing the symptoms that result from cancer (e.g., metastatic cancer or relapsed cancer), reducing or preventing the occurrence of such symptoms (either quantitatively or qualitatively), reducing the severity of such symptoms or physiological effects thereof, and/or enhancing the recovery of the individual after experiencing cancer (e.g., metastatic cancer or relapsed cancer) symptoms.

Specifically, the therapies (e.g., one or more oligonucleotide) of the present invention, when administered to an individual, can treat or prevent one or more of the symptoms or conditions associated with cancer (e.g., metastatic cancer or relapsed cancer) and/ or reduce or alleviate symptoms of or conditions associated with this disorder. As such, protecting an individual from the effects or symptoms resulting from cancer (e.g., metastatic cancer or relapsed cancer) includes both preventing or reducing the occurrence and/or severity of the effects of the disorder and treating a patient in which the effects of the disorder are already occurring or beginning to occur. A beneficial effect can easily be assessed by one of ordinary skill in the art and/or by a trained clinician who is treating the patient. Preferably, there is a positive or beneficial difference in the severity or occurrence of at least one clinical or biological score, value, or measure used to evaluate such individual in those who have been treated with the methods of the present invention as compared to those that have not. For example, the at least one clinical or biological score, value, or measure used to evaluate such an individual is the capability of cells (e.g., cancer stem cells) taken from a primary tumor, a secondary tumor, a biopsy, or ascites fluid of an individual to form spheres in a sphere formation assay. Methods for purifying cells such as cancer stem cells from tumors or other biological samples are well known in the art such as in U.S. Pat. No. 8,614,095, the disclosure of which is incorporated by reference herein in its entirety. In an exemplary sphere formation assay, a tumor is surgically removed from a subject and minced with a scalpel into fragments of approximately 2 to 3 mm3. The fragments are washed with a buffer (e.g., PBS) and then incubated with buffer containing sodium hypochlorite. The tumor tissue fragments are washed with buffer and digested with PBS and digested with a medium containing one or more of collagenase 1, collagenase IV, dispase, hyaluronidase and DNAase. The cell suspension is centrifuged and the pellet is suspended in buffer containing βFGF and EGF. The cells are washed to remove serum and suspended in medium supplemented with human EGF, human βFGF, B27 supplement without vitamin A, hydrocortisone, insulin, and N2 supplement. The cells are subsequently cultured in non-adherent plates. After 10 days in culture, spheres of 100 to 200 μm in diameter are obtained and counted. The spheres can be further expanded clonally, or injected into a subject to observe the capability of tumor formation. In some embodiments, an individual that has received an effective amount of one or more oligonucleotide (or compositions thereof) disclosed herein, alone or in combination with at least one therapy disclosed herein, has reduced sphere formation as compared to an individual not treated with the oligonucleotides of the present invention.

### VI. Articles of Manufacture or Kits

In another aspect, an article of manufacture or kit is provided wchich comprises one or more oligonucleotide described herein. The article of manufacture or kit may further comprise instructions for use of the one or more oligonucleotide in the methods of the invention. Accordingly, in certain embodiments, the article of manufacture or kit comprises instructions for use of one or more oligonucleotide complementary to an antisense non-coding chimeric mitochondrial RNA (ASncmtRNA) molecule or a sense non-coding chimeric mitochondrial RNA (SncmtRNA) molecule in methods for suppressing metastasis of a cancer, preventing or treating relapse of a cancer, and/or treating metastatic cancer in an individual comprising administering to the individual an effective amount of the one or more oligonucleotide. In certain embodiments, the individual has been previously treated for cancer with a therapy (e.g., chemotherapy, radiation therapy, surgery, or combinations thereof). In some embodiments, the article of manufacture or kit comprises instructions for use of one or more oligonucleotide complementary to an antisense non-coding chimeric mitochondrial RNA (ASncmtRNA) molecule or a sense non-coding chimeric mitochondrial RNA (SncmtRNA) molecule in methods for treating a refractory cancer (e.g., a refractory HPV-associated cancer) in an individual comprising administering to the individual an effective amount of the one or more oligonucleotide.

The article of manufacture or kit may further comprise a container. Suitable containers include, for example, bottles, vials (e.g., dual chamber vials), syringes (e.g., single or dual chamber syringes), IV bags, and test tubes. The container may be formed from a variety of materials such as glass or plastic. The container holds the composition (e.g., pharmaceutical formulation).

The article of manufacture or kit may further comprise a label or package insert, which is on, or associated with the container and may indicate directions for reconstitution and/or use of the composition (e.g., pharmaceutical formulation). The label may further indicate that the formulation is useful or intended for intravenous, subcutaneous, or other modes of administration for suppressing metastasis of a cancer, preventing or treating relapse of a cancer, and/or treating metastatic cancer in an individual. In other embodiments, the label may further indicate that the formulation is useful or intended for intravenous, subcutaneous, or other modes of administration for treating a refactory cancer (e.g., a refractory HPV-associated cancer) in an individual. The container holding the formulation may be a single-use vial or a multi-use vial, which allows for repeat administrations (e.g., from 2-6 administrations) of the reconstituted composition (e.g., pharmaceutical formulation). The article of manufacture or kit may further comprise a second container comprising a suitable diluent. The article of manufacture or kit may further include other materials desirable from a commercial, therapeutic, and user standpoint, including other buffers, diluents, filters, needles, syringes, and package inserts with instructions for use.

The article of manufacture of kit described herein optionally further comprises a container comprising a second therapeutic composition (e.g., an anti-cancer agent). For example, the article of manufacture or kit can comprise one or more oligonucleotide as a first composition (e.g., a first pharmaceutical composition) and an anti-cancer agent as a second composition (e.g., a second pharmaceutical composition). In some embodiments, the kit further comprises instructions for use of the one or more oligonucleotide in combination with the anti-cancer agent in the methods of the invention. An exemplary anti-cancer agents may be remicade, docetaxel, celecoxib, melphalan, dexamethasone, steroids, gemcitabine, cisplatinum, temozolomide, etoposide, cyclophosphamide, temodar, carboplatin, procarbazine, gliadel, tamoxifen, topotecan, methotrexate, gefitinib, taxol, taxotere, fluorouracil, leucovorin, irinotecan, xeloda, CPT-11, interferon alpha, pegylated interferon alpha, capecitabine, cisplatin, thiotepa, fludarabine, carboplatin, liposomal daunorubicin, cytarabine, doxetaxol, pacilitaxel, vinblastine, IL-2, GM-CSF, dacarbazine, vinorelbine, zoledronic acid, palmitronate, biaxin, busulphan, prednisone, bortezomib, bisphosphonate, arsenic trioxide, vincristine, doxorubicin, paclitaxel, ganciclovir, adriamycin, estrainustine sodium phosphate, sulindac, and/or etoposide.

### VII. Additional Exemplary Embodiments

The present application in some embodiments provides a method of treatment for a refractory cancer in an individual comprising administering to the individual an effective amount of one or more oligonucleotide complementary to an antisense non-coding chimeric mitochondrial RNA (ASncmtRNA) molecule or a sense non-coding chimeric mitochondrial RNA (SncmtRNA) molecule, wherein the oligonucleotide is able to hybridize with the chimeric mitochondrial RNA molecules to form a stable duplex.

In some embodiments according to (or as applied to) any of the embodiments above, the oligonucleotide is sufficiently complementary to a human non-coding chimeric mitochondrial RNA molecule comprising: a) an antisense 16S mitochondrial ribosomal RNA covalently linked at its 5′ end to the 3′ end of a polynucleotide with an inverted repeat sequence or b) a sense 16S mitochondrial ribosomal RNA covalently linked at its 5′ end to the 3′ end of a polynucleotide with an inverted repeat sequence.

In some embodiments according to (or as applied to) any of the embodiments above, the oligonucleotide is complementary to the ASncmtRNA molecule encoded by a nucleotide sequence selected from the group consisting of SEQ ID NO:4, SEQ NO:5, and SEQ NO:6.

In some embodiments according to (or as applied to) any of the embodiments above, the oligonucleotide is at least 85% complementary to the ASncmtRNA molecule encoded by a nucleotide sequence selected from the group consisting of SEQ ID NO:4, SEQ ID NO:5, and SEQ ID NO:6.

In some embodiments according to (or as applied to) any of the embodiments above, the one or more oligonucleotide comprises a nucleotide sequence selected from the group consisting of SEQ ID NOs:7-198.

In some embodiments according to (or as applied to) any of the embodiments above, the one or more oligonucleotide comprises a nucleic acid sequence selected from the group consisting of SEQ ID NOs:36, 197 and 198.

In some embodiments according to (or as applied to) any of the embodiments above, the oligonucleotide is administered in combination with at least one anti-cancer agent.

In some embodiments according to (or as applied to) any of the embodiments above, the oligonucleotide and the at least one anti-cancer agent is administered sequentially.

In some embodiments according to (or as applied to) any of the embodiments above, the oligonucleotide and the at least one anti-cancer agent is administered simultaneously.

In some embodiments according to (or as applied to) any of the embodiments above, the oligonucleotide is administered in combination with a radiation therapy.

In some embodiments according to (or as applied to) any of the embodiments above, the oligonucleotide is administered in combination with surgery.

In some embodiments according to (or as applied to) any of the embodiments above, the individual has been previously treated for cancer with a therapy comprising chemotherapy, radiation therapy, surgery, or combinations thereof.

In some embodiments according to (or as applied to) any of the embodiments above, the refractory cancer is a refractory HPV-associated cancer.

In some embodiments according to (or as applied to) any of the embodiments above, the refractory HPV-associated cancer is one or more selected from the group consisting of: cervical cancer, oralpharyngeal cancer, and head and neck cancer.

In some embodiments according to (or as applied to) any of the embodiments above, the refractory HPV-associated cancer is resistant to a chemotherapeutic agent.

In some embodiments according to (or as applied to) any of the embodiments above, the chemotherapeutic agent is one or more selected from the group consisting of: cisplatin, paclitaxel, actinomycin D, doxrubucin, etoposide, and 5-fluorouracil.

In some embodiments according to (or as applied to) any of the embodiments above, the refractory HPV-associated cancer is from an infection with one or more HPV strains selected from the group consisting of: HPV 16, HPV 18, HPV 31 and HPV 45.

In some embodiments according to (or as applied to) any of the embodiments above, the oligonucleotide reduces the number of cancer stem cells in the individual as compared to an individual not administered the oligonucleotide.

In some embodiments according to (or as applied to) any of the embodiments above, the oligonucleotide inhibits tumor growth and/or metastasis in the individual as compared to an individual not administered the oligonucleotide.

The invention will be more fully understood by reference to the following examples. The examples, which are intended to be purely exemplary of the invention, should not be construed as limiting the scope of the invention in any way. It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1

### Treatment of HCT-116 Colon Cancer Cells and Primary Cultures of Human Colon Cancer Cells with Antisense Oligonucleotides Complementary to Antisense Non-Coding Chimeric Mitochondrial RNA Abolished Sphere Formation

In this study, the ability of cells, from both the HCT-116 colon cancer cell line and primary cultures of human colon cancer cells derived from patients, to form spheroid bodies following treatment with antisense oligonucleotides directed to antisense non-coding chimeric mitochondrial RNA (ASncmtRNA) was determined. The assay utilized measured the numbers of spheroid bodies, also referred to herein as spheres, based on the specific ability of cancer stern cells to form these spheroid bodies.

Materials and Methods

### Experimental Scheme:

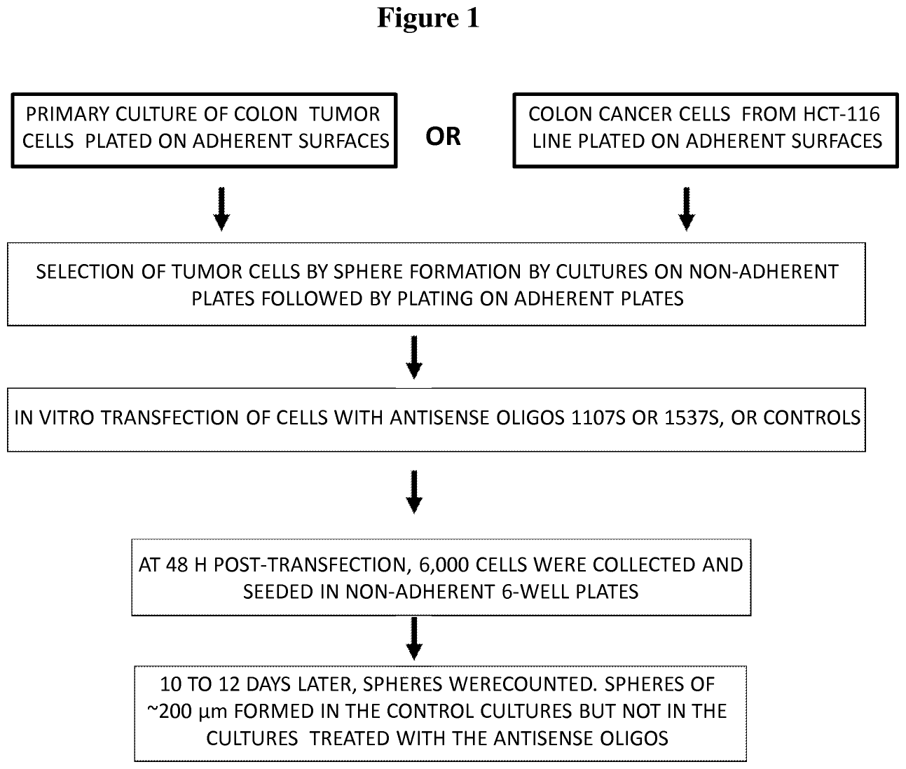

FIG. 1 demonstrates a general scheme of the experimental procedure and the assay utilized herein to measure the effect of antisense oligonucleotides targeted to ASncmtRNA on the number of spheres formed in colon cancer cells, based on the specific ability of cancer stem cells to form these spheroid bodies. Sphere formation was measured in HCT-116 colon cancer cells and primary cultures of human colon cancer cells derived from patients.

### Primary Cultures of Human Cancer Cells:

Biopsies of colon cancer were received post-surgery and with corresponding informed consent of each patient. Pieces of tumors of about 500 mm3 were transferred to sterile tubes of 50 ml containing DMEM medium. High-Glucose, GlataMax™ (GIBCO), 10% FCS (Biological Industries), Fungizone® 1×, 2× antibiotic-antimycotic mix and gentamicin 25 μg/ml (Invitrogen). The biopsies were processed within 2 to 3 h post-surgery.

In order to disaggregate the tumor, a piece of colon tumor was minced with a scalpel into fragments of approximately 2 to 3 mm3. The fragments were washed twice with PBS and then incubated for 20 minutes with PBS containing 0.5% sodium hypochlorite. The fragments were washed three times with PBS and digested with RPMI medium (Invitrogen) containing 1 mg/ml collagenase I, 2 mg/ml collagenase IV, 1 mg/ml dispase, 20 μg/ml hyaluronidase and 2000 U/ml DNAase. The mix was incubated at 37° C. for 60 min and with constant stirring. Next, the cell suspension was centrifuged at 200×g for 5 minutes and the pellet was suspended in PBS and centrifuged again a 200×g for 5 minutes. The pellet was resuspended in DMEM/F12 containing 1× N2 supplement, 10 ng/ml βFGF and 20 ng/ml EGF, for the formation of spheres.

### Culture of HCT-116 Cells:

HCT-116 cells were cultured according to standard protocols. HCT-116 cells obtained from ATCC were grown in DMEM medium containing penicillin, gentamicin and fungizone, with 10% FCS, at 37° C. and with 5% CO2.

### Selection of Tumor Cells by Sphere Formation and Adherence to Coated Plates:

Cells derived from colon cancer tumors or HCT-116 cultures were collected and washed to remove serum and suspended in serum-free DMEM/F12 supplemented with 100 IU/ml penicillin, 100 μg/ml streptomycin, 20 ng/ml human EGF, 20 ng/ml human βFGF, 2% B27 supplement without vitamin A, hydrocortisone, insulin (Lonza) and N2 supplement (Invitrogen, Carlsbad, Calif., USA). The cells were subsequently cultured in non-adherent plates (Corning Inc., Corning, N.Y., USA) at a density of about 5,000 cells/well or 1×105 cells in T25 flasks (Corning Inc. T25 3815). After 10 days in culture, spheres of 100 to 200 μm in diameter were obtained. The medium containing the spheres were filtered using a 70 μm nylon filter to eliminate single cells. The spheres were collected and dissociated with trypsin-EDTA and mechanically disrupted with a pipette. The cells were then centrifuged to remove the enzyme, washed once with DMEM medium containing 105 CFS and plated in adherent plates coated with collagen I (Gibco) and cultured at 37° C. and 5% CO2 as described before.

### Cell Transfection and Antisense Oligonucleotides:

Antisense oligonucleotides (ASOs) used in this study were synthesized by IDT (Integrated DNA Technologies, USA), Invitrogen or Biosearch Inc. with 100% phosphorothioate (PS) internucleosidic linkages. For transfection, cells were seeded into 12-well plates (Nunc) at 50,000 cells/well. The next day, cells selected from colon cancer tumors or from HCT-116 cultures (see Selection of tumor cells by sphere formation and adherence to coated plates) were transfected with the antisense oligonucleotides (ASO 1107S: 5′-GTCCTAAACTACCAAACC-3′ (SEQ ID NO:197) or ASO 1537S: 5′-CACCCACCCAAGAACAGG-3′ (SEQ ID NO:36), depending on the cell type) or a control oligonucleotide (Control Oligo 154: 5′-AGGTGGAGTGGATTGGGG-3′ (SEQ ID NO:199)) at a final concentration of 100 to 200 nM (depending on the cell type) using Lipofectamine 2000 (Invitrogen) according to the manufacturer's directions. In addition, a subset of cells were left untreated or in the presence of only Lipofectamine 2000. Transfection was for 48 hours under normal culture conditions.

### Sphere Formation Assay:

At 48 hours post transfection, the cells were harvested, counted and 5,000 to 6,000 cells were cultured in non-adherent 6-well plates (Corning Inc., Corning, N.Y., USA) as described before (described supra). After 10 days in culture, spheres of 100 to 200 μm in diameter were obtained and counted.

Results

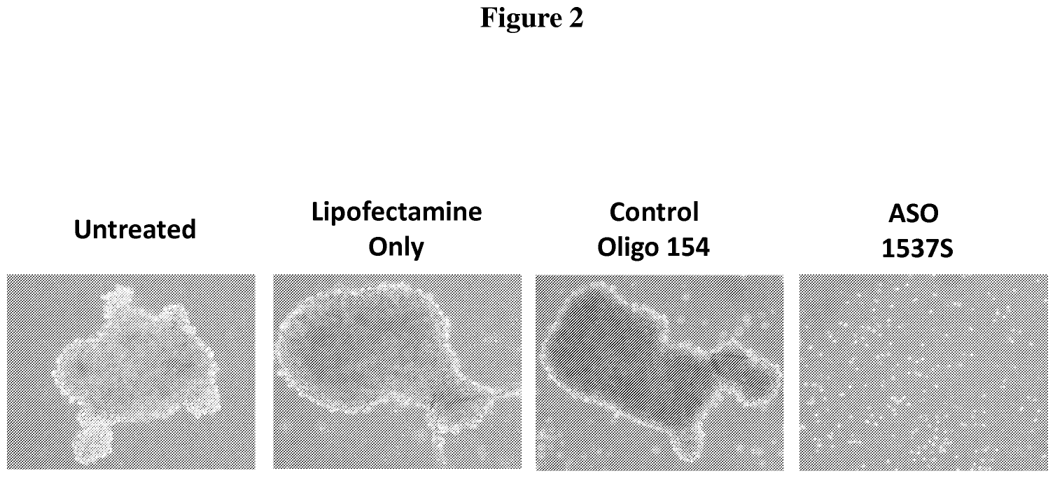

Sphere formation was unchanged in primary cultures of colon tumor cells following no treatment, treatment with only Lipofectamine, or treatment with Control Oligo 154. But sphere formation was abolished in these primary cells following treatment with ASO 1537S (FIG. 2).

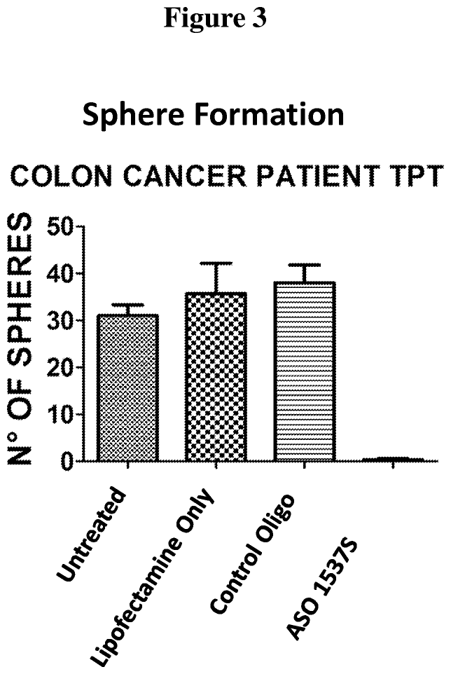

A primary culture of a colon tumor (patient TPT; 50,000 cells) was used to quantify sphere formation and evaluate the effect of transfection with ASO 1537S on the capacity of these cells to form spheres. The three groups of control cells (untreated, treated with Lipofectamine only, or treated with Control Oligo 154) formed spheres (30 to 37 spheres) equivalent to approximately 0.6% of the total number of cells seeded. Cells transfected with ASO 1537S were unable to form spheres (FIG. 3).

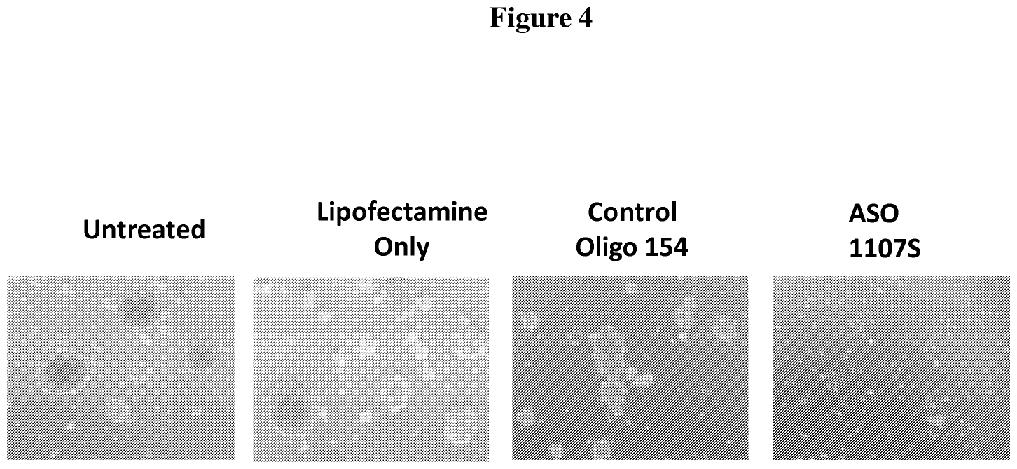

Sphere formation was unchanged in cells from the HCT-116 colon tumor cell line following no treatment, treatment with only Lipofectamine, or treatment with Control Oligo 154. However, sphere formation was abolished in these cells following treatment with ASO 1107S (FIG. 4).

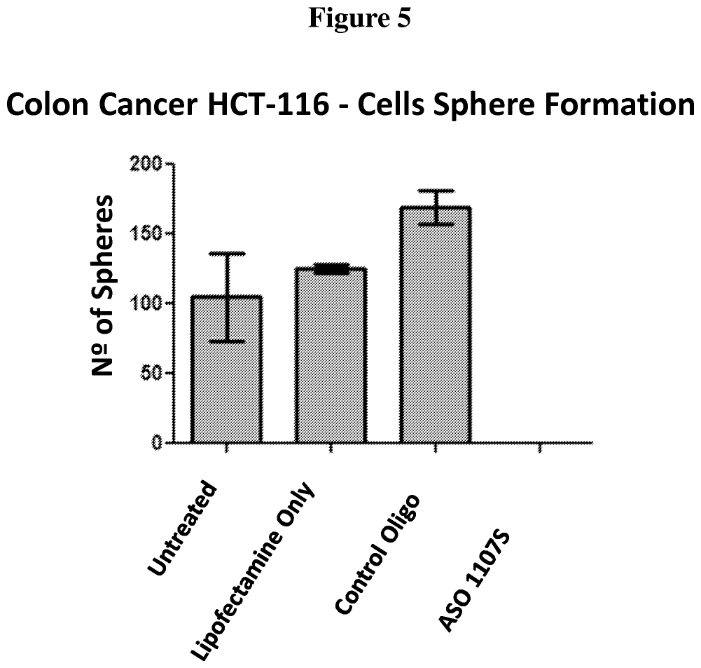

The colon tumor cell line HCT-116 was used to quantify sphere formation and evaluate the effect of transfection with ASO 1107S on sphere formation. The three groups of control cells (untreated, treated with Lipofectamine only, or treated with Control Oligo 154) formed spheres (100 to 160 spheres), equivalent to 0.3% of the total amount of cells seeded (in this representative example 50,000 cells in T25 flasks). However, cells transfected with ASO 1107S were not able to form spheres (FIG. 5).

Taken together, these results show that groups left untreated, treated with only Lipofectamine, or transfected with a control oligonucleotide retained the ability to form spheres. In contrast, primary colon tumor cells or FICT-116 colon cancer cells transfected with antisense oligonucleotides lost their ability to form spheres. These results indicate that the antisense oligonucleotides were able to kill cancer stem cells as indicated by the lack of spehere formation upon treatment. Furthermore, these results also indicate that only a fraction of all cells seeded were able to form spheres.

### Example 2

### Treatment of a Cervical Cancer Cell Line and Primary Cultures of Human Uterine Cervical Cancer Cells with Antisense Oligonucleotides Complementary to Antisense Non-Coding Chimeric Mitochondrial RNA Abolished Sphere Formation

The ability of cervical cells from the SiHa cervical cancer cell line (transformed with Human Papillomavirus 16 or HPV 16) and from primary cultures of human uterine cervical cancer cells derived from patients to form spheroid bodies following treatment with antisense oligonucleotides directed to antisense non-coding chimeric mitochondrial RNA (ASnscmtRNA) was determined. The assay utilized measured the numbers of spheroid bodies, also referred to herein as spheres, which ared formed by cancer stem cells.

Materials and Methods

### Experimental Scheme:

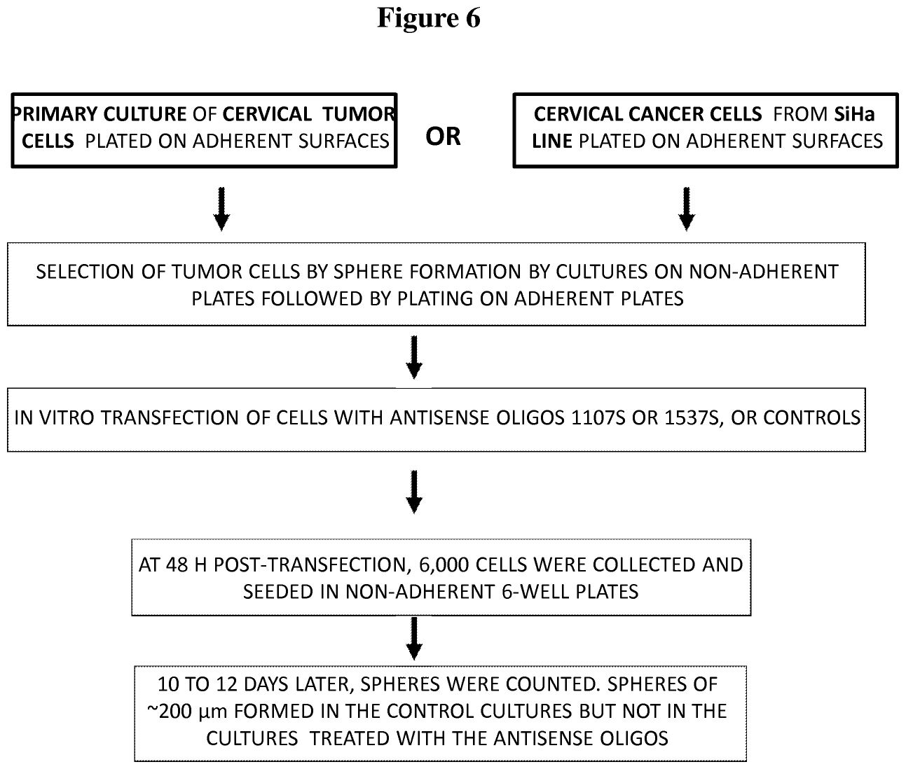

The experimental procedure and assay utilized herein measured the effect of antisense oligonucleotides that were targeted to ASncmtRNA on the number of spheres formed by cervical cancer cells (FIG. 6). Sphere formation was measured in the SiHa cervical cancer cell line and primary cultures of human cervical cancer cells derived from patients (CerCa). CerCa 1, CerCa 2 and CerCa 3 (transformed with Human Papillomavirus 45) were obtained from patient biopsies.

### Culture of SiHa Cells:

SiHa cells were grown in DMEM, “High Glucose”, GlutaMAX™, containing penicillin, gentamicin and fungizone, with 10% FCS at 37° C. and with 5% CO2.

### Primary Cultures of Human Cancer Cells:

Biopsies of uterine cervical cancer were received post-surgery and with the corresponding informed consent of each patient. Pieces of tumors of about 500 mm3 were transferred to 50 mL sterile tubes containing DMEM medium, High-Glucose, GlataMax™ (GIBCO), 10% FCS (Biological Industries), Fungizone® 1×, 2× antibiotic-antimycotic mix and gentamicin 25 μg/ml (Invitrogen). The biopsies were processed within 2 to 3 hhours post-surgery.

In order to disaggregate the tumor, the tissue sample was placed on a 10 cm Petri dish with 10 ml of sterile PBS (Gibco) and sliced with a scalpel into small pieces (1-3 mm3). The pieces were transfered to 6 cm dishes together with 2 ml of medium containing 0.5% Colagenase I, 0.5% Colagenase II, 0.5% Colagenase IV (GIBCO), 0.5% hialorunidase (AppliChem), 0.1% Dispase (GIBCO), 0.05% DNase (AppliChem), 0.1% BSA (Rockland), antibiotic-antimycotic 2× mix (GIBCO) and Fungizone® 2× (GIBCO). The fragments were incubated at 37° C. for 30 min and the cell suspension was centrifuged at 300×g for 5 minutes. The pellet was suspended in 5 ml MEGM™ medium containing 20 ng/ml of hEGF 20, hydrocortisone, insulin, GA-1000 (Lonza), 0.5× B-27 without vitamin A (Invitrogen), 20 ng/ml of FGFb (Invitrogen) and 5% CFB. The cells were cultured in a T25 flask coated with collagen (BD Bioscience) at 37° C. and with 5% CO2. The medium was changed every 48 hours.

### Selection of Tumor Cells by Sphere Formation and Adherence to Coated Plates:

About 1×1.05 cells resuspended in MEGM™ medium supplemented with 20 ng/ml of hEGF, hydrocortisone, insulin, GA-1000 (Lonza), 0.5× B-27 without vitamin A (Invitrogen), 20 ng/ml of FGFb (Invitrogen) without CRS were seeded on ultra-low adherence plates (Corning). Ten days later, the spheres were counted under phase microscopy, collected and filtered using a nylon mesh of 70 μm to discard single cells. The spheres were recovered from the filter and seeded on 6-well plates (Corning) previously coated with collagen type I (Gibco) and cultured as described above. Three human primary cultures of cervical tumors, CerCa 1, CerCa 2 and CerCa 3, were isolated.

### Human Papillomavirus (HPV) Genotyping:

The different primary cultures were analyzed with the PGMY09/11 Linear Array (Roche). The presence of HPV 16 was detected in CerCa 1 and CerCa 2 primary cell cultures while HPV 45 was detected in the CerCa 3 primary cell culture.

### Cell Transfection and Antisense Oligonucleotides:

Antisense oligonucleotides (ASOs) used in this study were synthesized by IDT (Integrated DNA Technologies, USA), Invitrogen or Biosearch Inc. with 100% phosphorothioate (PS) internucleosidic linkages. For transfection, cells were seeded into 12-well plates (Nunc) at 5000 cells/well. The next day, cells selected from cervical cancer tumors or from SiHa cells (See Selection of Tumor Cells by Sphere Formation and Adherence to Coated Plates) were transfected with an antisense oligonucleotide (ASO 1537S: 5′-CACCCACCCAAGAACAGG-3′ (SEC) ID NO:36), depending of the cell type) or a control oligonucleotide 154 (ASO-C: 5′-AGGTGGAGTGGATTGGGG-3′(SEQ ID NO:199)) at a final concentration of 100 nM (SiHa cells) or 200 nM (CerCa cells) using Lipofectamine 2000 (Invitrogen) according to the manufacturer's directions. In addition, a subset of cells were left untreated (NT), transfected in the presence of Lipofectamine 2000 (LIPO) only, or incubated with 45 uM cisplatin (OSP). Transfection was conducted for 72 hours under normal culture conditions.

### Sphere Formation Assay:

At 72 hours post transfection, the cells were harvested, counted and 5,000 to 6,000 cells were cultured in non-adherent 6-well plates (Corning Inc., Corning, N.Y., USA) as described above. After 10 days in culture, spheres of 100 to 200 μm in diameter were obtained and counted.

Results

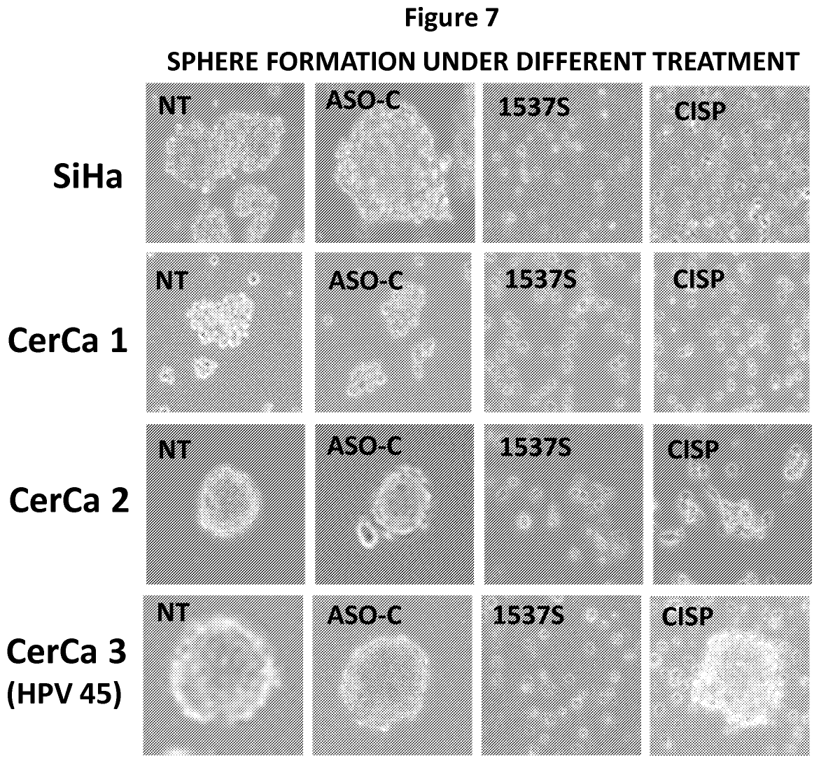

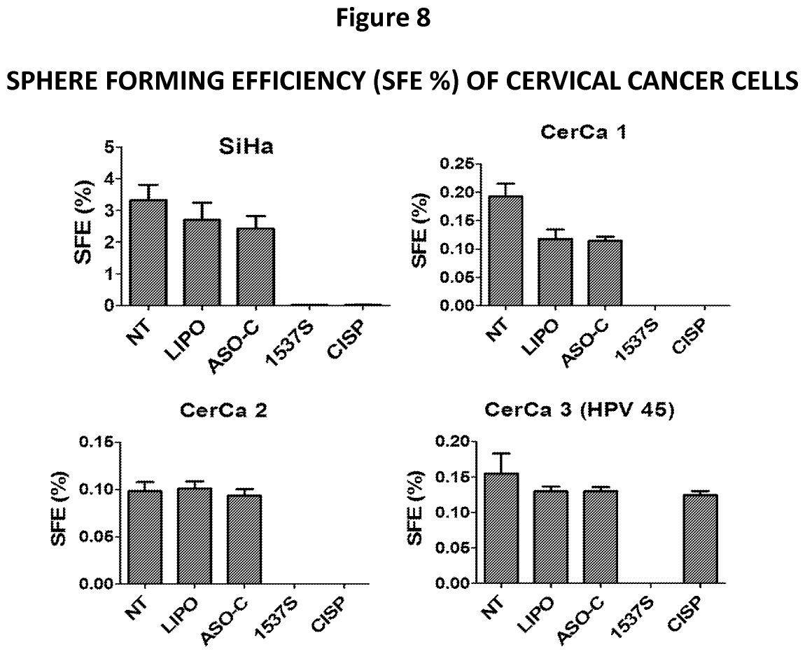

Sphere formation was unchanged in primary cultures of cervical tumor cells following no treatment (NT), treatment with only Lipofectamine (LIPO), or treatment with control oligonucleotide 154 (ASO-C). In contrast, sphere formation was abolished in SiHa, CerCa 1, CerCa 2 and CerCa 3 primary cultures following treatment with ASO 1537S (FIG. 7 and FIG. 8). Sphere formation of SiHa, CerCa 1 and CerCa 2 cells (all infected with HPV 16) was also abolished when cells were treated with the drug cisplatin (CISP) (45 μM) (FIG. 7 and FIG. 8) while no effect was observed in CerCa 3 cells (infected with HPV 45) treated with cisplatin.

Taken together, these results show that cervical cancer primary cultures (CerCa 1, CerCa 2 and CerCa 3 cells) and the cell line SiHa left untreated, treated with only Lipofectamine, or transfected with a control oligonucleotide retained the ability to form spheres. In contrast, primary cervical tumor cells or SiHa cells transfected with antisense oligonucleotides targeted to the antisense non-coding chimeric mitochondrial RNA (ASncmtRNA) molecules or sense non-coding chimeric mitochondrial RNA (SncmtRNA) molecule, lost their ability to form spheres whether HPV 16 or HPV 45 positive. These results indicate that the antisense oligonucleotides were able to kill cervical cancer stem cells as indicated by the lack of sphere formation upon treatment. Moreover, treatment with the anti-cancer drug cisplatin abolished sphere formation of SiHa, CerCa 1 and CerCa 2 cells (all HPV 16 positive) but not CerCa 3 that is infected with HPV 45 (FIG. 7 and FIG. 8). Therefore, these results indicate that the antisense oligonuleotides are able to kill cervical cancer stem (CerCa 3 cells) resistant to cisplatin treatment. See Tjalme et al., Am. J. Clin. Pathol., 137:161, 2012; de Sanjosé et al., Eur J Cancer., 49(16): 3450, 2013; Tjalma et al., Int J Cancer., 132(4):854, 2013.

### Example 3

### Treatment of Mice with Antisense Oligonucleoitides Complementary to the Antisense Non-Coding Chimeric Mitochondrial RNA Following Surgery to Remove Intradermal Melanoma Tumors Prevented Relapse of Tumor Growth and Metastasis in the Lungs and Liver

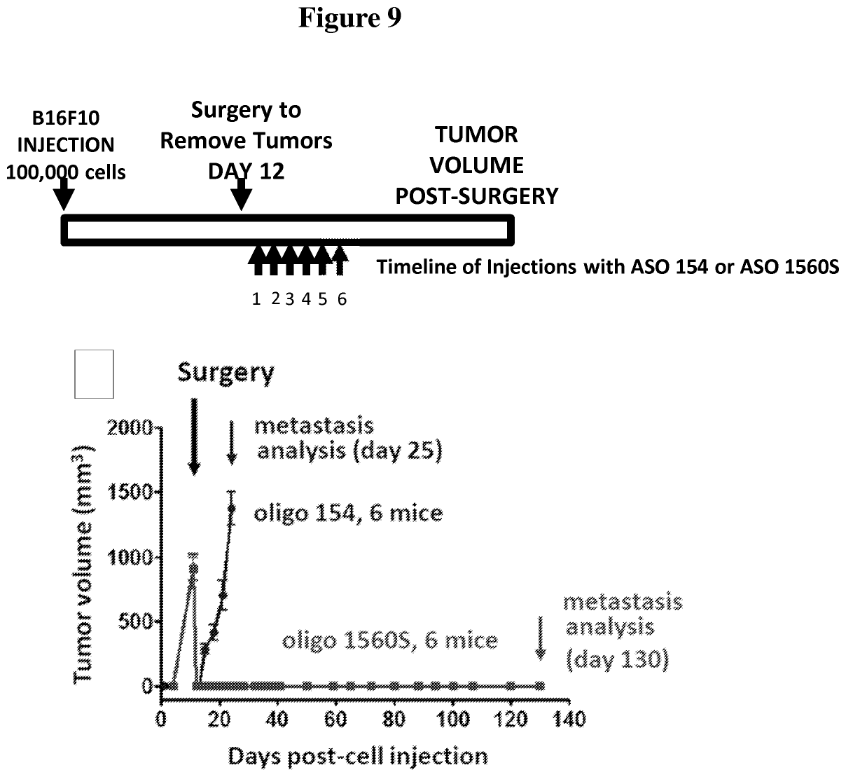

One common clinical protocol for melanoma includes surgical resection follow by systemic administration of drugs. Similar protocols are used in the practice of other tumors. In the melanoma model presented in this representative example, B16F10 melanoma cells (100,000 cells in 200 μl of saline) were injected subcutaneously on the back of C57BL/6 mice. About 11 to 12 days post-cell injection, tumors between 700 to 1,000 mm3 developed (a 1,000 mm3 tumor in mice is considered equivalent to a 3,000 cc3 tumor in humans). At this time, mice were randomly divided in two groups (Control Oligo ASO 154 and ASO 1560S (SEQ ID NO:198)) with similar tumor volume. Tumors were surgically resected under anesthesia and the wound washed once with 250 μl containing 100 μg of ASO 1560S or ASO 154. After surgical suture, a bolus of 200 μl saline containing 100 μg of ASO 154 or ASO 1560S was applied into the cavity left by the tumor. Three days post-surgery, mice received on alternative days, 3 intravenous (FIG. 9; 1st, 3rd, 5th arrows on timeline) or 3 intraperitoneal (FIG. 9; 2nd, 4th, 6th arrows on timeline) injections of 250 μl saline containing 100 μg of either ASO 1560S or ASO 154. Tumor growth was measured twice a week with a caliper.

Relapse of tumor growth in mice treated with ASO 154 was observed about 3 days post-surgery and tumor volume of about 1,500 mm3 was reached on about the 25th day post-cell injection (FIG. 9). These mice were euthanized under anesthesia, and the tumor and other organs were fixed and saved for further studies. No relapse was observed in mice treated with ASO 1560S targeted to the mouse ASncmtRNAs. At 130 days post-cell injection, mice appeared healthy without the presence of detectable tumors and were euthanized to collect organs. Livers and lungs were analyzed for the presence of metastatic nodules.

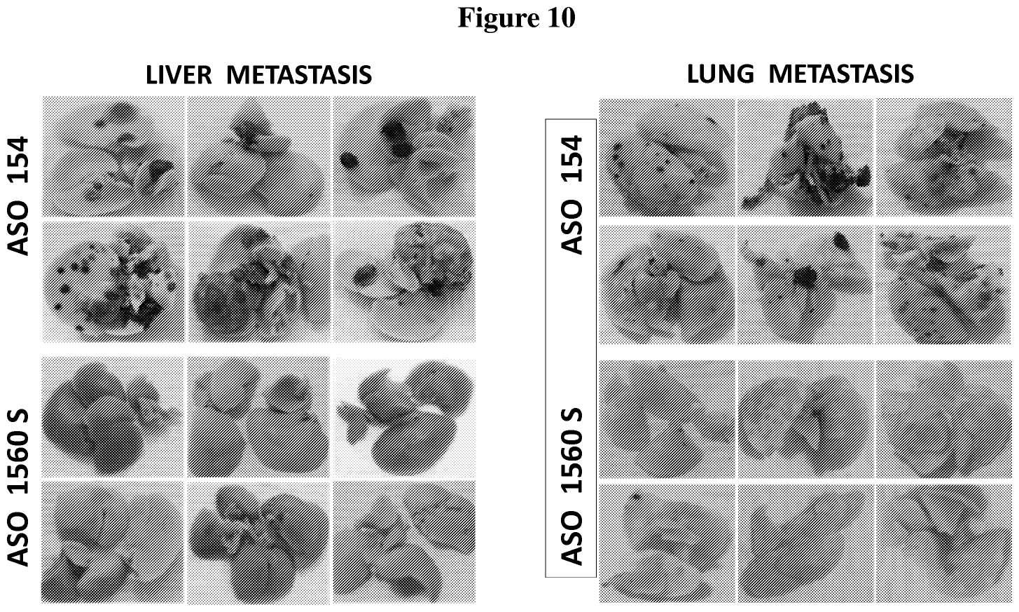

Control mice (treated with Control Oligo ASO 154) showed the presence of cancer relapse and metastatic black nodules in the lung and liver. In contrast, the lungs and livers of the mice treated with ASO 1560S lacked the presence of metastatic nodules demonstrating that ASO 1560S prevented or suppressed cancer relapse (FIG. 10).

### Example 4

### Intravenous Treatment of Mice with Antisense Oligonucleoitides Complementary to the Antisense Non-Coding Chimeric Mitochondrial RNA Following Surgery to Remove Intradermal Kidney Tumors Resulted in Absence of Tumor Relapse and Complete Survival

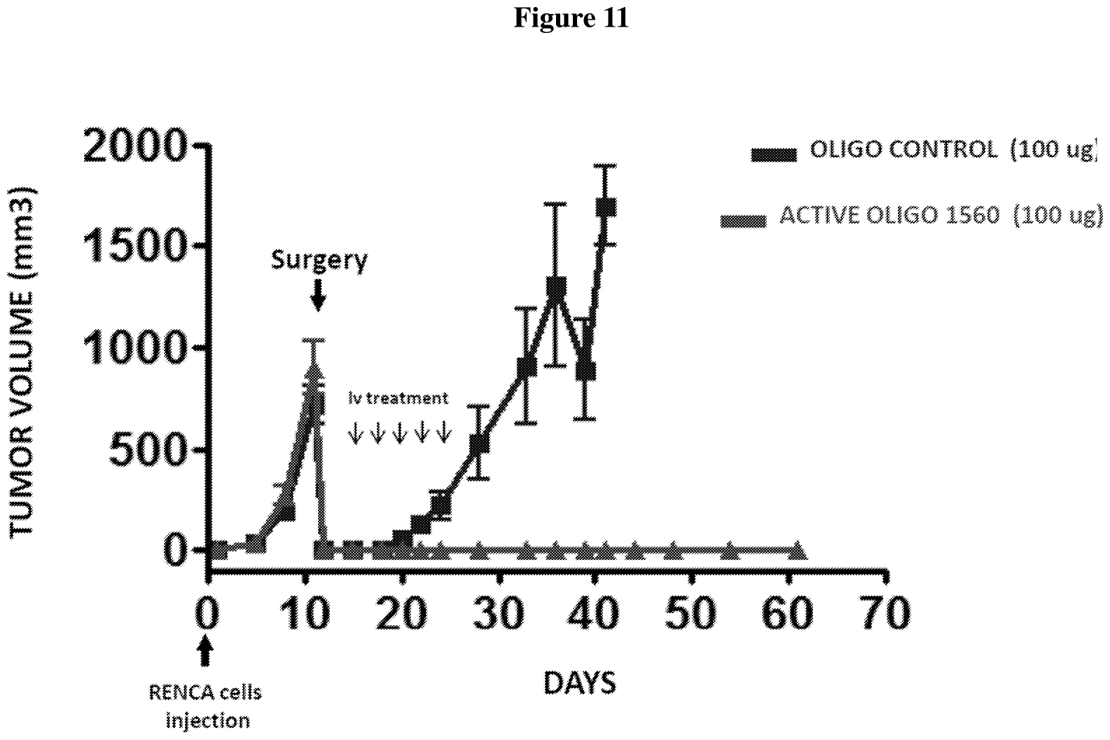

100,000 RENCA cells (ATCC® CRL-2947™ mus musculus kidney renal adenocarcina) were injected subcutaneously at day 0. At day 12, tumors of an average size of 800 mm3 were removed and the site of the tumor was washed with 100 μg of Control Oligo ASO 154 (4 animals) or ASO1560S (5 animals) in 200 μl, both formulated in liposomes. The animals were sutured and intravenously injected at the site of the removed tumor with 100 μg of ASO 154 (FIG. 11, squares) or ASO 1560S (FIG. 11, triangles), both formulated in liposomes, in 250 μl. No further treatment was given. At day 40, (30 days post-surgery), all the control animals had died with tumors of an average size of 1,200 mm3 and extensive metastasis, while all animals treated with ASO 1560S had no tumors and were still alive at day 61 (FIG. 11).

### Example 5

### Intraperitoneal Treatment of Mice with Antisense Oligonucleoitides Complementary to the Antisense Non-Coding Chimeric Mitochondrial RNA Following Surgery to Remove Intradermal Kidney Tumors Resulted in Absence of Tumor Relapse and Complete Survival

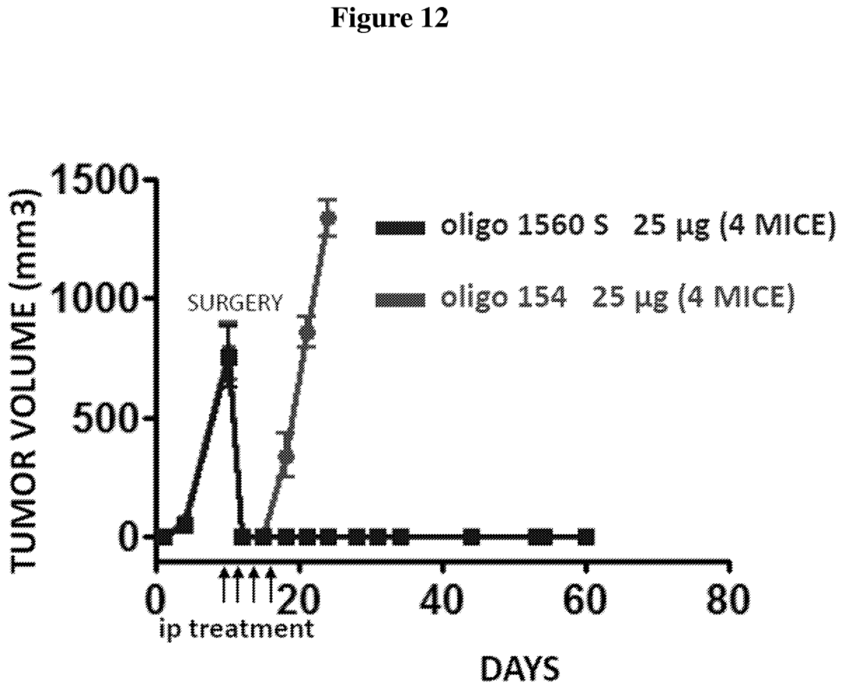

100,000 RENCA cells (ATCC® CRL-2947™ mus musculus kidney renal adenocarcina) were injected subcutaneously on day 0 in 8 mice. On day 11, tumors had an average size of 800 mm3. Tumors of all animals were removed by surgery and divided in 2 groups. The wound of the control group was washed once with Control Oligo ASO 154 before suturing and the wound of the treated group was washed with ASO 1560S before suturing. Post-suture, a bolus of 250 μl was intraperiotoneally injected in the place of where tumor had grown: the control group was injected with ASO 154 and the treated group with ASO 1560S. On days 13, 15, 17 and 19, intraperitoneal injections of 25 μg ASO 154 (FIG. 12, circles) or 25 μg ASO 1560S (FIG. 12, squares) formulated in liposomes were injected in a volume of 250 μl. On days 14, 16 and 18, the mice were injected intravenously with the same protocol. On day 22, all control animals had tumors larger than 1,200 mm3 and were sacrificed. On day 60, all animals treated with ASO 1560S had no tumors and were still alive (FIG. 12).

### Example 6

### Treatment of Mice with Antisense Oligonucleoitides Complementary to the Antisense Non-Coding Chimeric Mitochondrial RNA Following Surgery to Remove Intradermal Melanoma Tumors Resulted in Absence of Tumor Relapse and Complete Survival

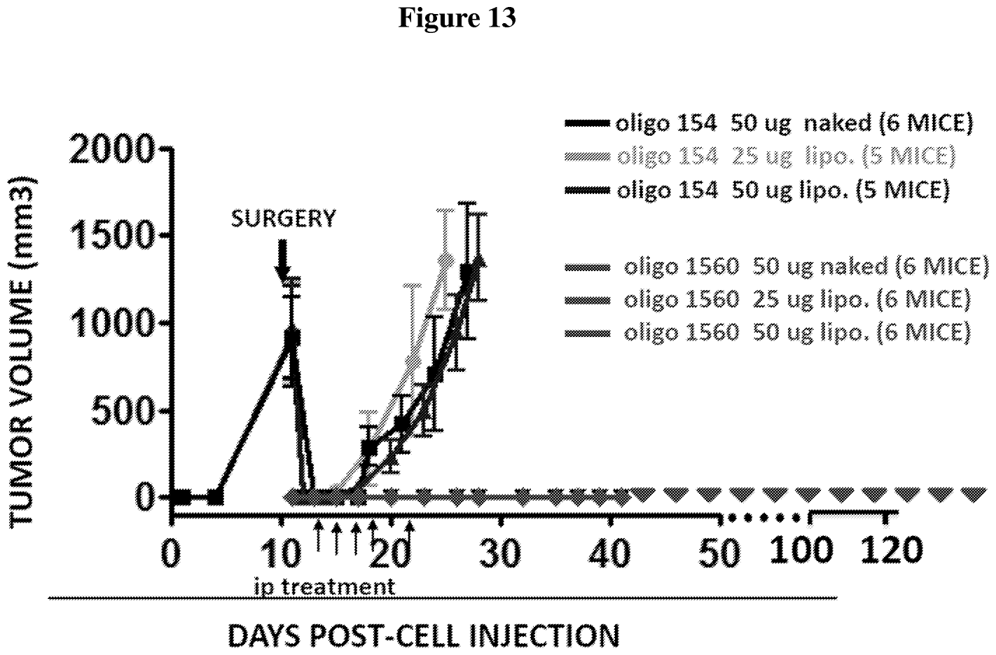

B 16F10 melanoma cells (100,000 cells in 200 μl of RPMI medium) were injected into mice subcutaneously on day 0. On day 11, surgery was carried out to remove tumors. The tumor volumes varied from approximately 800 to 1200 mm3. The wound was washed once. Post-suture one bolus of 250 μl of oligo in liposomes was injected in the tumor site. On days 13, 15, 17, 19 and 21, a dose of 25 μg control ASO 154 naked (FIG. 13, squares), 25 μg of control ASO 154 in liposomes (FIG. 13, circles), or 50 μg of ASO 154 in liposomes (FIG. 13, triangles), each in a volume of 250 μl, was intraperitoneally injected in the mice. Other groups of mice (6 mice per group) were injected in a similar manner as described above with 50 μg of ASO 1560 naked, 25 μg of ASO 1560S in liposomes or 50 μg of ASO 1560S in liposomes (FIG. 13, diamonds).

Compared to mice treated with a control oligonucleotide, post-surgery treatment with 25 and 50 μg intraperitoneal injections of ASO 1560S resulted in the absence of the intradermal melanoma tumor relapse and complete survival (FIG. 13).

### Example 7

### Intraperitoneal or Intravenous Treatment of Mice with Antisense Oligonucleotides Complementary to the Antisense Non-Coding Chimeric Mitochondrial RNA Following Surgery to Remove Subcutaneous Bladder Carcinoma Tumors Resulted in Absence of Tumor and Complete Survival

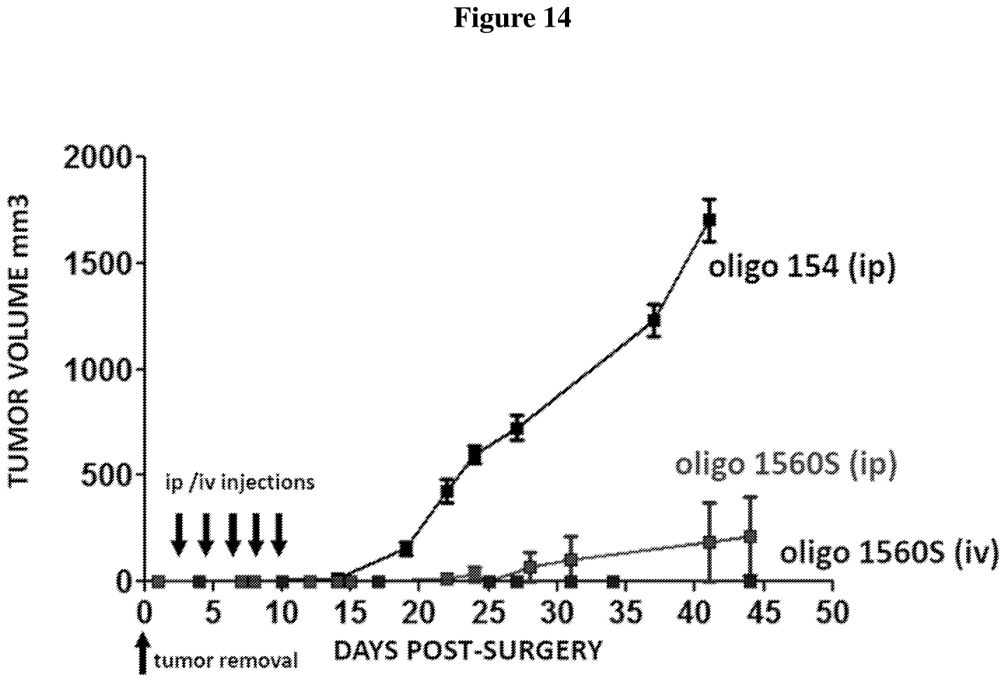

Twelve mice were injected subcutaneously with a 100,000 MB49 cells (mouse bladder cancer cells). After 15 days mice had tumors of an average diameter of 800 mm3. Tumors were surgically removed (day 0) and a bolus of 200 μl containing 100 μg of ASO 154 (control) or ASO 1560S (active drug) was injected at the site of the surgery. Three days post-surgery mice were divided in 2 groups and treated intraperitoneally or intravenously with 100 μl injections containing ASO 154 (control group) or ASO 1560S (treated group) as indicated in FIG. 14. Only one mouse treated intraperitoneally with ASO 1560S developed a tumor. All the mice treated intravenously with ASO 1560S remained without tumors and experienced full survival. Control animals treated with Control Oligo ASO 154 were sacrificed at day 41 (FIG. 14).

### Example 8

### Treatment of Rag −/− Mice with Antisense Oligonucleotides Complementary to the Antisense Non-Coding Chimeric Mitochondrial RNA Following Tumor Removal of a Human Melanoma Resulted in Significant Elimination of Tumor Relapse and a Large Increase in Survival

Rag −/− mice were injected with 5 million human A375 melanoma cells. Approximately at 34 days post cell-injection, all mice developed tumors of about 700 mm3. Mice were subjected to tumor removal surgery and divided randomly in two groups.

Group 1 (control): the wound was washed with 50 μg of Control Oligo 154 (6 mice in group) in a volume of 200 μl. Group 2 (therapy): the wound was washed with 50 μg of ASO 1537S (8 mice in group) (Sequence of the oligonucleotide provided in Example 1) in 200 μl. After suturing, a bolus of 250 μl containing 50 μg of Control Oligo 154 (6 control mice) or 250 μl containing 50 g of ASO Oligo 1537S was applied (8 therapy mice). Two days later mice received 6 injections with Control Oligo 154 or ASO Oligo 1537S every other day. The first injection was intraperitoneal and the second injection was in the tail vein.

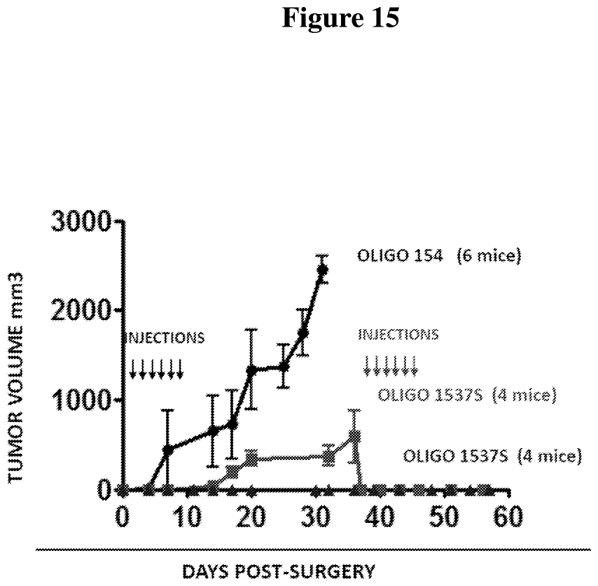

On day 30 after surgery, the 6 mice treated with Control Oligo 154 had tumors on the order of 2000 mm3 and were sacrificed (FIG. 15, circles). On day 57 after surgery, 4 out of the 8 mice receiving ASO Oligo 1537S were without tumors (FIG. 15, triangles). On day 17 post surgery, 4 out of the 8 mice receiving ASO Oligo 1537S began to show small tumors that grew slowly which at day 36 reached a size of about 500 mm3 (FIG. 15, squares). These mice were again subjected to surgery to remove the tumors, and subsequently 2 mice died. The other 2 mice were subjected to the same therapeutic protocol (alternating 3 injections intraperitoneal and 3 injections intravenous) with 50 μg of ASO Oligo 1537S in 250 μl (FIG. 15).

1.-64. (canceled)

65. A method for treating or suppressing metastatic cancer in an individual comprising administering to the individual an effective amount of one or more oligonucleotide complementary to an antisense non-coding chimeric mitochondrial RNA (ASncmtRNA) molecule or a sense non-coding chimeric mitochondrial RNA (SncmtRNA) molecule, wherein the one or more oligonucleotide is able to hybridize with the chimeric mitochondrial RNA molecules to form a stable duplex, wherein the individual has been previously treated for cancer with a therapy comprising chemotherapy, radiation therapy, surgery, or combinations thereof.

66. The method of claim 65, wherein the oligonucleotide is sufficiently complementary to a human non-coding chimeric mitochondrial RNA molecule comprising:
a. an antisense 16S mitochondrial ribosomal RNA covalently linked at its 5′ end to the 3′ end of a polynucleotide with an inverted repeat sequence; or
b. a sense 16S mitochondrial ribosomal RNA covalently linked at its 5′ end to the 3′ end of a polynucleotide with an inverted repeat sequence.

67. The method of claim 65, wherein the individual has been previously treated for cancer with a therapy and said therapy comprises chemotherapy.

68. The method of claim 65, wherein the individual has been previously treated for cancer with a therapy and said therapy comprises surgery.

69. The method of claim 65, wherein the individual has been previously treated for cancer with a therapy and said therapy comprises radiation therapy.

70. The method of claim 65, wherein the one or more oligonucleotide is complementary to the antisense 16S mitochondrial ribosomal RNA molecule encoded by a nucleotide sequence selected from the group consisting of SEQ ID NO: 203, SEQ ID NO: 204, SEQ ID NO: 205.

71. The method of claim 65, wherein the wherein the metastatic cancer is selected from the group consisting of bladder cancer, brain cancer, breast cancer, cervical cancer, colon cancer, endometrial cancer, esophageal cancer, gastric cancer, liver and bile duct cancer, lung cancer, melanoma, oral cancer, ovarian cancer, pancreatic cancer, pharynx cancer, prostate cancer, renal cancer, testicular cancer, or thyroid cancer.

72. The method of claim 65, wherein the one or more oligonucleotide comprises a nucleotide sequence selected from the group consisting of SEQ ID NOs: 7-198.

73. The method of claim 65, wherein the one or more oligonucleotide comprises a nucleotide sequence of SEQ ID NO: 36, 54, 61, or 65.

74. The method of claim 65, wherein the one or more oligonucleotide comprises a nucleotide sequence of SEQ ID NO: 70.

75. The method of claim 65, wherein the one or more oligonucleotide comprises a nucleotide sequence of SEQ ID NO: 74.

76. The method of claim 65, wherein the one or more oligonucleotide comprises a nucleotide sequence of SEQ ID NO: 76.

77. The method of claim 65, wherein the one or more oligonucleotide comprises a nucleotide sequence of SEQ ID NO: 79.

78. The method of claim 65, wherein the one or more oligonucleotide comprises a nucleotide sequence of SEQ ID NO: 81.

79. The method of claim 65, wherein the one or more oligonucleotide comprises a nucleotide sequence of SEQ ID NO: 88.

80. The method of claim 65, wherein the one or more oligonucleotide comprises a nucleotide sequence of SEQ ID NO : 197.

81. The method of claim 65, wherein the one or more oligonucleotide comprises a nucleotide sequence of SEQ ID NO: 198.

82. The method of claim 65, wherein the oligonucleotide is administered in combination with at least one anti-cancer agent.

83. The method of claim 82, wherein the at least one anti-cancer agent is selected from the group consisting of remicade, docetaxel, celecoxib, melphalan, dexamethasone, steroids, gemcitabine, cisplatinum, temozolomide, etoposide, cyclophosphamide, temodar, carboplatin, procarbazine, gliadel, tamoxifen, topotecan, methotrexate, gefitinib, taxol, taxotere, fluorouracil, leucovorin, irinotecan, xeloda, CPT-11, interferon alpha, pegylated interferon alpha, capecitabine, cisplatin, thiotepa, fludarabine, carboplatin, liposomal daunorubicin, cytarabine, doxetaxol, pacilitaxel, vinblastine, IL-2, GM-CSF, dacarbazine, vinorelbine, zoledronic acid, palmitronate, biaxin, busulphan, prednisone, bortezomib, bisphosphonate, arsenic trioxide, vincristine, doxorubicin, paclitaxel, ganciclovir, adriamycin, estrainustine sodium phosphate, sulindac, and etoposide.

84. A kit comprising one or more oligonucleotides complementary to an antisense non-coding chimeric mitochondrial RNA (ASncmtRNA) molecule or a sense non-coding chimeric mitochondrial RNA (SncmtRNA) molecule and instructions for practicing the methods of claim 65.


SEQUENCE LISTING (205 sequences)

SEQ ID NO: 1 - DNA, 2374 bp, Homo sapiens
tctaatactggtgatgctagaggtgatgtttttggtaaacaggcggggtaagatttgccgagttccttttactttttttaacctttccttatgagcatgcctgtgttgggttgacagtgagggtaataatgacttgttggttgattgtagatattgggctgttaattgtcagttcagtgttttaatctgacgcaggcttatgcggaggagaatgttttcatgttacttatactaacattagttcttctatagggtgatagattggtccaattgggtgtgaggagttcagttatatgtttgggattttttaggtagtgggtgttgagcttgaacgctttcttaattggtggctgcttttaggcctactatgggtgttaaattttttactctctctacaaggttttttcctagtgtccaaagagctgttcctctttggactaacagttaaatttacaaggggatttagagggttctgtgggcaaatttaaagttgaactaagattctatcttggacaaccagctatcaccaggctcggtaggtttgtcgcctctacctataaatcttcccactattttgctacatagacgggtgtgctcttttagctgttcttaggtagctcgtctggtttcgggggtcttagctttggctctccttgcaaagttatttctagttaattcattatgcagaaggtataggggttagtccttgctatattatgcttggttataatttttcatctttcccttgcggtactatatctattgcgccaggtttcaatttctatcgcctatactttatttgggtaaatggtttggctaaacctagccccaaacccactccaccttactaccagacaaccttagccaaaccatttacccaaataaagtataggcgatagaaattgaaacctggcgcaatagatatagtaccgcaagggaaagatgaaaaattataaccaagcataatatagcaaggactaacccctataccttctgcataatgaattaactagaaataactttgcaaggagagccaaagctaagacccccgaaaccagacgagctacctaagaacagctaaaagagcacacccgtctatgtagcaaaatagtgggaagatttataggtagaggcgacaaacctaccgagcctggtgatagctggttgtccaagatagaatcttagttcaactttaaatttgcccacagaaccctctaaatccccttgtaaatttaactgttagtccaaagaggaacagctctttggacactaggaaaaaaccttgtagagagagtaaaaaatttaacacccatagtaggcctaaaagcagccaccaattaagaaagcgttcaagctcaacacccactacctaaaaaatcccaaacatataactgaactcctcacacccaattggaccaatctatcaccctatagaagaactaatgttagtataagtaacatgaaaacattctcctccgcataagcctgcgtcagattaaaacactgaactgacaattaacagcccaatatctacaatcaaccaacaagtcattattaccctcactgtcaacccaacacaggcatgctcataaggaaaggttaaaaaaagtaaaaggaactcggcaaatcttaccccgcctgtttaccaaaaacatcacctctagcatcaccagtattagaggcaccgcctgcccagtgacacatgtttaacggccgcggtaccctaaccgtgcaaaggtagcataatcacttgttccttaaatagggacctgtatgaatggctccacgagggttcagctgtctcttacttttaaccagtgaaattgacctgcccgtgaagaggcgggcataacacagcaagacgagaagaccctatggagctttaatttattaatgcaaacagtacctaacaaacccacaggtcctaaactaccaaacctgcattaaaaatttcggttggggcgacctcggagcagaacccaacctccgagcagtacatgctaagacttcaccagtcaaagcgaactactatactcaattgatccaataacttgaccaacggaacaagttaccctagggataacagcgcaatcctattctagagtccatatcaacaatagggtttacgacctcgatgttggatcaggacatcccgatggtgcagccgctattaaaggttcgtttgttcaacgattaaagtcctacgtgatctgagttcagaccggagtaatccaggtcggtttctatctaccttcaaattcctccctgtacgaaaggacaagagaaataaggcctacttcacaaagcgccttcccccgtaaatgatatcatctcaacttagtattatacccacacccacccaagaacagggttt

SEQ ID NO: 2 - DNA, 1679 bp, Homo sapiens
ggggtcttagctttggctctccttgcaaagttatttctagttaattcattatgcagaaggtataggggttagtccttgctatattatgcttggttataatttttcatctttcccttgcggtgctaaacctagccccaaacccactccaccttactaccagacaaccttagccaaaccatttacccaaataaagtataggcgatagaaattgaaacctggcgcaatagatatagtaccgcaagggaaagatgaaaaattataaccaagcataatatagcaaggactaacccctataccttctgcataatgaattaactagaaataactttgcaaggagagccaaagctaagacccccgaaaccagacgagctacctaagaacagctaaaagagcacacccgtctatgtagcaaaatagtgggaagatttataggtagaggcgacaaacctaccgagcctggtgatagctggttgtccaagatagaatcttagttcaactttaaatttgcccacagaaccctctaaatccccttgtaaatttaactgttagtccaaagaggaacagctctttggacactaggaaaaaaccttgtagagagagtaaaaaatttaacacccatagtaggcctaaaagcagccaccaattaagaaagcgttcaagctcaacacccactacctaaaaaatcccaaacatataactgaactcctcacacccaattggaccaatctatcaccctatagaagaactaatgttagtataagtaacatgaaaacattctcctccgcataagcctgcgtcagattaaaacactgaactgacaattaacagcccaatatctacaatcaaccaacaagtcattattaccctcactgtcaacccaacacaggcatgctcataaggaaaggttaaaaaaagtaaaaggaactcggcaaatcttaccccgcctgtttaccaaaaacatcacctctagcatcaccagtattagaggcaccgcctgcccagtgacacatgtttaacggccgcggtaccctaaccgtgcaaaggtagcataatcacttgttccttaaatagggacctgtatgaatggctccacgagggttcagctgtctcttacttttaaccagtgaaattgacctgcccgtgaagaggcgggcataacacagcaagacgagaagaccctatggagctttaatttattaatgcaaacagtacctaacaaacccacaggtcctaaactaccaaacctgcattaaaaatttcggttggggcgacctcggagcagaacccaacctccgagcagtacatgctaagacttcaccagtcaaagcgaactactatactcaattgatccaataacttgaccaacggaacaagttaccctagggataacagcgcaatcctattctagagtccatatcaacaatagggtttacgacctcgatgttggatcaggacatcccaatggtgcagccgctattaaaggttcgtttgttcaacgattaaagtcctacgtgatctgagttcagaccggagtaatccaggtcggtttctatctacttcaaattcctccctgtacgaaaggacaagagaaataaggcctacttcacaaagcgccttcccccgtaaatgatatcatctcaacttagtattatacccacacccacccaagaacagggttt

SEQ ID NO: 3 - DNA, 1635 bp, Homo sapiens
ggggtcttagctttggctctccttgcaaagttatttctagttaattcattatgcagaaggtataggggttagtccttgctaaacctagccccaaacccactccaccttactaccagacaaccttagccaaaccatttacccaaataaagtataggcgatagaaattgaaacctggcgcaatagatatagtaccgcaagggaaagatgaaaaattataaccaagcataatatagcaaggactaacccctataccttctgcataatgaattaactagaaataactttgcaaggagagccaaagctaagacccccgaaaccagacgagctacctaagaacagctaaaagagcacacccgtctatgtagcaaaatagtgggaagatttataggtagaggcgacaaacctaccgagcctggtgatagctggttgtccaagatagaatcttagttcaactttaaatttgcccacagaaccctctaaatccccttgtaaatttaactgttagtccaaagaggaacagctctttggacactaggaaaaaaccttgtagagagagtaaaaaatttaacacccatagtaggcctaaaagcagccaccaattaagaaagcgttcaagctcaacacccactacctaaaaaatcccaaacatataactgaactcctcacacccaattggaccaatctatcaccctatagaagaactaatgttagtataagtaacatgaaaacattctcctccgcataagcctgcgtcagattaaaacactgaactgacaattaacagcccaatatctacaatcaaccaacaagtcattattaccctcactgtcaacccaacacaggcatgctcataaggaaaggttaaaaaaagtaaaaggaactcggcaaatcttaccccgcctgtttaccaaaaacatcacctctagcatcaccagtattagaggcaccgcctgcccagtgacacatgtttaacggccgcggtaccctaaccgtgcaaaggtagcataatcacttgttccttaaatagggacctgtatgaatggctccacgagggttcagctgtctcttacttttaaccagtgaaattgacctgcccgtgaagaggcgggcataacacagcaagacgagaagaccctatggagctttaatttattaatgcaaacagtacctaacaaacccacaggtcctaaactaccaaacctgcattaaaaatttcggttggggcgacctcggagcagaacccaacctccgagcagtacatgctaagacttcaccagtcaaagcgaactactatactcaattgatccaataacttgaccaacggaacaagttaccctagggataacagcgcaatcctattctagagtccatatcaacaatagggtttacgacctcgatgttggatcaggacatcccaatggtgcagccgctattaaaggttcgtttgttcaacgattaaagtcctacgtgatctgagttcagaccggagtaatccaggtcggtttctatctacttcaaattcctccctgtacgaaaggacaagagaaataaggcctacttcacaaagcgccttcccccgtaaatgatatcatctcaacttagtattatacccacacccacccaagaacagggttt

SEQ ID NO: 4 - DNA, 1921 bp, Homo sapiens
aacctccgagcagtacatgctaagacttcaccagtcaaagcgaactactatactcaattgatccaataacttgaccaacggaacaagttaccctagggataacagcgcaatcctattctagagtccatatcaacaatagggtttacgacctcgatgttggatcaggacatcccaatggtgcagccgctattaaaggttcgtttgttcaacgattaaagtcctacgtgatctgagttcagaccggagtaatccaggtcggtttctatctacttcaaattcctccctgtacgaaaggacaagagaaataaggcctacttcacaaagcgccttcccccgtaaatgatatcatctcaacttagtattataccctgttcttgggtgggtgtgggtataatactaagttgagatgatatcatttacgggggaaggcgctttgtgaagtaggccttatttctcttgtcctttcgtacagggaggaatttgaagtagatagaaaccgacctggattactccggtctgaactcagatcacgtaggactttaatcgttgaacaaacgaacctttaatagcggctgcaccatcgggatgtcctgatccaacatcgaggtcgtaaaccctattgttgatatggactctagaataggattgcgctgttatccctagggtaacttgttccgttggtcaagttattggatcaattgagtatagtagttcgctttgactggtgaagtcttagcatgtactgctcggaggttgggttctgctccgaggtcgccccaaccgaaatttttaatgcaggtttggtagtttaggacctgtgggtttgttaggtactgtttgcattaataaattaaagctccatagggtcttctcgtcttgctgtgttatgcccgcctcttcacgggcaggtcaatttcactggttaaaagtaagagacagctgaaccctcgtggagccattcatacaggtccctatttaaggaacaagtgattatgctacctttgcacggttagggtaccgcggccgttaaacatgtgtcactgggcaggcggtgcctctaatactggtgatgctagaggtgatgtttttggtaaacaggcggggtaagatttgccgagttccttttactttttttaacctttccttatgagcatgcctgtgttgggttgacagtgagggtaataatgacttgttggttgattgtagatattgggctgttaattgtcagttcagtgttttaatctgacgcaggcttatgcggaggagaatgttttcatgttacttatactaacattagttcttctatagggtgatagattggtccaattgggtgtgaggagttcagttatatgtttgggattttttaggtagtgggtgttgagcttgaacgctttcttaattggtggctgcttttaggcctactatgggtgttaaattttttactctctctacaaggttttttcctagtgtccaaagagctgttcctctttggactaacagttaaatttacaaggggatttagagggttctgtgggcaaatttaaagttgaactaagattctatcttggacaaccagctatcaccaggctcggtaggtttgtcgcctctacctataaatcttcccactattttgctacatagacgggtgtgctcttttagctgttcttaggtagctcgtctggtttcgggggtcttagctttggctctccttgcaaagttatttctagttaattcattatgcagaaggtataggggttagtccttgctatattatgcttggttataatttttcatctttcccttgcggtactatatctattgcgccaggtttcaatttctatcgcctatactttatttgggtaaatggtttggctaaggttgtctggtagtaaggtggagtgggtttggggctaggtttagc

SEQ ID NO: 5 - DNA, 1744 bp, Homo sapiens
tagggataacagcgcaatcctattctagagtccatatcaacaatagggtttacgacctcgatgttggatcaggacatcccgatggtgcagccgctattaaaggttcgtttgttcaacgattaaagtcctacgtgatctgagttcagaccggagtaatccaggtcggtttctatctaccttcaaattcctccctgttcttgggtgggtgtgggtataatactaagttgagatgatatcatttacgggggaaggcgctttgtgaagtaggccttatttctcttgtcctttcgtacagggaggaatttgaagtagatagaaaccgacctggattactccggtctgaactcagatcacgtaggactttaatcgttgaacaaacgaacctttaatagcggctgcaccatcgggatgtcctgatccaacatcgaggtcgtaaaccctattgttgatatggactctagaataggattgcgctgttatccctagggtaacttgttccgttggtcaagttattggatcaattgagtatagtagttcgctttgactggtgaagtcttagcatgtactgctcggaggttgggttctgctccgaggtcgccccaaccgaaatttttaatgcaggtttggtagtttaggacctgtgggtttgttaggtactgtttgcattaataaattaaagctccatagggtcttctcgtcttgctgtgttatgcccgcctcttcacgggcaggtcaatttcactggttaaaagtaagagacagctgaaccctcgtggagccattcatacaggtccctatttaaggaacaagtgattatgctacctttgcacggttagggtaccgcggccgttaaacatgtgtcactgggcaggcggtgcctctaatactggtgatgctagaggtgatgtttttggtaaacaggcggggtaagatttgccgagttccttttactttttttaacctttccttatgagcatgcctgtgttgggttgacagtgagggtaataatgacttgttggttgattgtagatattgggctgttaattgtcagttcagtgttttaatctgacgcaggcttatgcggaggagaatgttttcatgttacttatactaacattagttcttctatagggtgatagattggtccaattgggtgtgaggagttcagttatatgtttgggattttttaggtagtgggtgttgagcttgaacgctttcttaattggtggctgcttttaggcctactatgggtgttaaattttttactctctctacaaggttttttcctagtgtccaaagagctgttcctctttggactaacagttaaatttacaaggggatttagagggttctgtgggcaaatttaaagttgaactaagattctatcttggacaaccagctatcaccaggctcggtaggtttgtcgcctctacctataaatcttcccactattttgctacatagacgggtgtgctcttttagctgttcttaggtagctcgtctggtttcgggggtcttagctttggctctccttgcaaagttatttctagttaattcattatgcagaaggtataggggttagtccttgctatattatgcttggttataatttttcatctttcccttgcggtactatatctattgcgccaggtttcaatttctatcgcctatactttatttgggtaaatggtttggctaaggttgtctggtagtaaggtggagtgggtttggggctaggtttagc

SEQ ID NO: 6 - DNA, 1854 bp, Homo sapiens
gaactcggcaaatcttaccccgcctgtttaccaaaaacatcacctctagcatcaccagtattagaggcaccgcctgcccagtgacacatgtttaacggccgcggtaccctaaccgtgcaaaggtagcataatcacttgttccttaaatagggacctgtatgaatggctccacgagggttcagctgtctcttacttttaaccagtgaaattgacctgcccgtgaagaggcgggcatgacacagcaagacgagaagaccctatggagctttaatttattaatgcaaacagtacctaacaaaccctgttcttgggtgggtgtgggtataatactaagttgagatgatatcatttacgggggaaggcgctttgtgaagtaggccttatttctcttgtcctttcgtacagggaggaatttgaagtagatagaaaccgacctggattactccggtctgaactcagatcacgtaggactttaatcgttgaacaaacgaacctttaatagcggctgcaccatcgggatgtcctgatccaacatcgaggtcgtaaaccctattgttgatatggactctagaataggattgcgctgttatccctagggtaacttgttccgttggtcaagttattggatcaattgagtatagtagttcgctttgactggtgaagtcttagcatgtactgctcggaggttgggttctgctccgaggtcgccccaaccgaaatttttaatgcaggtttggtagtttaggacctgtgggtttgttaggtactgtttgcattaataaattaaagctccatagggtcttctcgtcttgctgtgttatgcccgcctcttcacgggcaggtcaatttcactggttaaaagtaagagacagctgaaccctcgtggagccattcatacaggtccctatttaaggaacaagtgattatgctacctttgcacggttagggtaccgcggccgttaaacatgtgtcactgggcaggcggtgcctctaatactggtgatgctagaggtgatgtttttggtaaacaggcggggtaagatttgccgagttccttttactttttttaacctttccttatgagcatgcctgtgttgggttgacagtgagggtaataatgacttgttggttgattgtagatattgggctgttaattgtcagttcagtgttttaatctgacgcaggcttatgcggaggagaatgttttcatgttacttatactaacattagttcttctatagggtgatagattggtccaattgggtgtgaggagttcagttatatgtttgggattttttaggtagtgggtgttgagcttgaacgctttcttaattggtggctgcttttaggcctactatgggtgttaaattttttactctctctacaaggttttttcctagtgtccaaagagctgttcctctttggactaacagttaaatttacaaggggatttagagggttctgtgggcaaatttaaagttgaactaagattctatcttggacaaccagctatcaccaggctcggtaggtttgtcgcctctacctataaatcttcccactattttgctacatagacgggtgtgctcttttagctgttcttaggtagctcgtctggtttcgggggtcttagctttggctctccttgcaaagttatttctagttaattcattatgcagaaggtataggggttagtccttgctatattatgcttggttataatttttcatctttcccttgcggtactatatctattgcgccaggtttcaatttctatcgcctatactttatttgggtaaatggtttggctaaggttgtctggtagtaaggtggagtgggtttggggctaggtttagc

SEQ ID NO: 7 - DNA, 20 bp, Artificial Sequence
  Antisense Oligonucleotide
taggtttagcaccgcaaggg

SEQ ID NO: 8 - DNA, 20 bp, Artificial Sequence
  Antisense Oligonucleotide
taggtttagcaaggactaac

SEQ ID NO: 9 - DNA, 18 bp, Artificial Sequence
  Antisense Oligonucleotide
ggggtaagatttgccgag

SEQ ID NO: 10 - DNA, 22 bp, Artificial Sequence
  Antisense Oligonucleotide
atgctagaggtgatgtttttgg

SEQ ID NO: 11 - DNA, 18 bp, Artificial Sequence
  Antisense Oligonucleotide
cggtgcctctaatactgg

SEQ ID NO: 12 - DNA, 20 bp, Artificial Sequence
  Antisense Oligonucleotide
gttaaacatgtgtcactggg

SEQ ID NO: 13 - DNA, 18 bp, Artificial Sequence
  Antisense Oligonucleotide
ttgcacggttagggtacc

SEQ ID NO: 14 - DNA, 21 bp, Artificial Sequence
  Antisense Oligonucleotide
ggaacaagtgattatgctacc

SEQ ID NO: 15 - DNA, 21 bp, Artificial Sequence
  Antisense Oligonucleotide
ggagccattcatacaggtccc

SEQ ID NO: 16 - DNA, 20 bp, Artificial Sequence
  Antisense Oligonucleotide
agtaagagacagctgaaccc

SEQ ID NO: 17 - DNA, 19 bp, Artificial Sequence
  Antisense Oligonucleotide
ggcaggtcaatttcactgg

SEQ ID NO: 18 - DNA, 19 bp, Artificial Sequence
  Antisense Oligonucleotide
gctgtgttatgcccgcctc

SEQ ID NO: 19 - DNA, 19 bp, Artificial Sequence
  Antisense Oligonucleotide
agctccatagggtcttctc

SEQ ID NO: 20 - DNA, 20 bp, Artificial Sequence
  Antisense Oligonucleotide
gttaggtactgtttgcatta

SEQ ID NO: 21 - DNA, 18 bp, Artificial Sequence
  Antisense Oligonucleotide
aagtcttagcatgtactg

SEQ ID NO: 22 - DNA, 19 bp, Artificial Sequence
  Antisense Oligonucleotide
tagtagttcgctttgactg

SEQ ID NO: 23 - DNA, 19 bp, Artificial Sequence
  Antisense Oligonucleotide
caagttattggatcaattg

SEQ ID NO: 24 - DNA, 18 bp, Artificial Sequence
  Antisense Oligonucleotide
gggtaacttgttccgttg

SEQ ID NO: 25 - DNA, 18 bp, Artificial Sequence
  Antisense Oligonucleotide
aataggattgcgctgtta

SEQ ID NO: 26 - DNA, 18 bp, Artificial Sequence
  Antisense Oligonucleotide
cctattgttgatatggac

SEQ ID NO: 27 - DNA, 17 bp, Artificial Sequence
  Antisense Oligonucleotide
ctgatccaacatcgagg

SEQ ID NO: 28 - DNA, 18 bp, Artificial Sequence
  Antisense Oligonucleotide
tagcggctgcaccattgg

SEQ ID NO: 29 - DNA, 19 bp, Artificial Sequence
  Antisense Oligonucleotide
gttgaacaaacgaaccttt

SEQ ID NO: 30 - DNA, 19 bp, Artificial Sequence
  Antisense Oligonucleotide
aactcagatcacgtaggac

SEQ ID NO: 31 - DNA, 18 bp, Artificial Sequence
  Antisense Oligonucleotide
cgacctggattactccgg

SEQ ID NO: 32 - DNA, 18 bp, Artificial Sequence
  Antisense Oligonucleotide
ggaatttgaagtagatag

SEQ ID NO: 33 - DNA, 19 bp, Artificial Sequence
  Antisense Oligonucleotide
ctcttgtcctttcgtacag

SEQ ID NO: 34 - DNA, 18 bp, Artificial Sequence
  Antisense Oligonucleotide
ggcgctttgtgaagtagg

SEQ ID NO: 35 - DNA, 22 bp, Artificial Sequence
  Antisense Oligonucleotide
gttgagatgatatcatttacgg

SEQ ID NO: 36 - DNA, 18 bp, Artificial Sequence
  Antisense Oligonucleotide
cacccacccaagaacagg

SEQ ID NO: 37 - DNA, 25 bp, Artificial Sequence
  Antisense Oligonucleotide
caacttagtattatacccacaccca

SEQ ID NO: 38 - DNA, 22 bp, Artificial Sequence
  Antisense Oligonucleotide
tcccccgtaaatgattacatct

SEQ ID NO: 39 - DNA, 25 bp, Artificial Sequence
  Antisense Oligonucleotide
gagaaataaggcctacttcacaaag

SEQ ID NO: 40 - DNA, 22 bp, Artificial Sequence
  Antisense Oligonucleotide
caaattcctccctgtacgaaag

SEQ ID NO: 41 - DNA, 24 bp, Artificial Sequence
  Antisense Oligonucleotide
agtaatccaggtcggtttctatct

SEQ ID NO: 42 - DNA, 24 bp, Artificial Sequence
  Antisense Oligonucleotide
aagtcctagctgatctgagttcag

SEQ ID NO: 43 - DNA, 25 bp, Artificial Sequence
  Antisense Oligonucleotide
gctattaaaggttcgtttgttcaac

SEQ ID NO: 44 - DNA, 16 bp, Artificial Sequence
  Antisense Oligonucleotide
tcccgatggtgcagcc

SEQ ID NO: 45 - DNA, 22 bp, Artificial Sequence
  Antisense Oligonucleotide
ttacgacctcgatgttggatca

SEQ ID NO: 46 - DNA, 25 bp, Artificial Sequence
  Antisense Oligonucleotide
atcctattctagagtccatatcaac

SEQ ID NO: 47 - DNA, 24 bp, Artificial Sequence
  Antisense Oligonucleotide
aataggattgcgctgttatcccta

SEQ ID NO: 48 - DNA, 24 bp, Artificial Sequence
  Antisense Oligonucleotide
tagggataacagcgcatacctatt

SEQ ID NO: 49 - DNA, 23 bp, Artificial Sequence
  Antisense Oligonucleotide
ggaacaagttaccctagggataa

SEQ ID NO: 50 - DNA, 23 bp, Artificial Sequence
  Antisense Oligonucleotide
ttgatccaataacttgaccaacg

SEQ ID NO: 51 - DNA, 21 bp, Artificial Sequence
  Antisense Oligonucleotide
acttcaccagtcaaagcgaac

SEQ ID NO: 52 - DNA, 18 bp, Artificial Sequence
  Antisense Oligonucleotide
aacccaacctccgagcag

SEQ ID NO: 53 - DNA, 16 bp, Artificial Sequence
  Antisense Oligonucleotide
gttggggcgacctcgg

SEQ ID NO: 54 - DNA, 22 bp, Artificial Sequence
  Antisense Oligonucleotide
aaactaccaaacctgcttaaaa

SEQ ID NO: 55 - DNA, 24 bp, Artificial Sequence
  Antisense Oligonucleotide
aaacagtacctaacaaacccacag

SEQ ID NO: 56 - DNA, 25 bp, Artificial Sequence
  Antisense Oligonucleotide
gaccctatggagctttaatttatta

SEQ ID NO: 57 - DNA, 23 bp, Artificial Sequence
  Antisense Oligonucleotide
cataacacagcaagacgagaaga

SEQ ID NO: 58 - DNA, 18 bp, Artificial Sequence
  Antisense Oligonucleotide
tgacctgcccgtgaagag

SEQ ID NO: 59 - DNA, 26 bp, Artificial Sequence
  Antisense Oligonucleotide
cagctgtctcttacttttaaccagtg

SEQ ID NO: 60 - DNA, 20 bp, Artificial Sequence
  Antisense Oligonucleotide
ctgtatgaatggctccacga

SEQ ID NO: 61 - DNA, 26 bp, Artificial Sequence
  Antisense Oligonucleotide
agcataatcacttgttccttaaatag

SEQ ID NO: 62 - DNA, 23 bp, Artificial Sequence
  Antisense Oligonucleotide
accgtgcaaaggtagcataatca

SEQ ID NO: 63 - DNA, 23 bp, Artificial Sequence
  Antisense Oligonucleotide
tgattatgctacctttgcacggt

SEQ ID NO: 64 - DNA, 19 bp, Artificial Sequence
  Antisense Oligonucleotide
gtaccctaaccgtgcaaag

SEQ ID NO: 65 - DNA, 21 bp, Artificial Sequence
  Antisense Oligonucleotide
cctgcccagtgacacatgttt

SEQ ID NO: 66 - DNA, 25 bp, Artificial Sequence
  Antisense Oligonucleotide
cacctctagcatcaccagtattaga

SEQ ID NO: 67 - DNA, 20 bp, Artificial Sequence
  Antisense Oligonucleotide
cttaccccgcctgtttacca

SEQ ID NO: 68 - DNA, 26 bp, Artificial Sequence
  Antisense Oligonucleotide
aggttaaaaaaagtaaaaggaactcg

SEQ ID NO: 69 - DNA, 19 bp, Artificial Sequence
  Antisense Oligonucleotide
cccaacacaggcatgctca

SEQ ID NO: 70 - DNA, 24 bp, Artificial Sequence
  Antisense Oligonucleotide
accaacaagtcattattaccctca

SEQ ID NO: 71 - DNA, 24 bp, Artificial Sequence
  Antisense Oligonucleotide
tgacaattaacagcccaatatcta

SEQ ID NO: 72 - DNA, 21 bp, Artificial Sequence
  Antisense Oligonucleotide
gcctgcgtcagattaaaacac

SEQ ID NO: 73 - DNA, 24 bp, Artificial Sequence
  Antisense Oligonucleotide
gtaacatgaaaacattctcctccg

SEQ ID NO: 74 - DNA, 28 bp, Artificial Sequence
  Antisense Oligonucleotide
tatcaccctatagaagaactaatgttag

SEQ ID NO: 75 - DNA, 21 bp, Artificial Sequence
  Antisense Oligonucleotide
ctgaactcctcacacccaatt

SEQ ID NO: 76 - DNA, 23 bp, Artificial Sequence
  Antisense Oligonucleotide
cactacctaaaaaatcccaaaca

SEQ ID NO: 77 - DNA, 21 bp, Artificial Sequence
  Antisense Oligonucleotide
ttaagaaagcgttcaagctca

SEQ ID NO: 78 - DNA, 21 bp, Artificial Sequence
  Antisense Oligonucleotide
catagtaggcctaaaagcagc

SEQ ID NO: 79 - DNA, 26 bp, Artificial Sequence
  Antisense Oligonucleotide
aaaccttgtagagagagtaaaaaatt

SEQ ID NO: 80 - DNA, 24 bp, Artificial Sequence
  Antisense Oligonucleotide
aaagaggaacagctctttggacac

SEQ ID NO: 81 - DNA, 24 bp, Artificial Sequence
  Antisense Oligonucleotide
aatccccttgtaaatttaactgtt

SEQ ID NO: 82 - DNA, 21 bp, Artificial Sequence
  Antisense Oligonucleotide
ctttaaatttgcccacagaac

SEQ ID NO: 83 - DNA, 20 bp, Artificial Sequence
  Antisense Oligonucleotide
ggttgtccaagatagaatct

SEQ ID NO: 84 - DNA, 19 bp, Artificial Sequence
  Antisense Oligonucleotide
acaaacctaccgagcctgg

SEQ ID NO: 85 - DNA, 20 bp, Artificial Sequence
  Antisense Oligonucleotide
aagatttataggtagaggcg

SEQ ID NO: 86 - DNA, 20 bp, Artificial Sequence
  Antisense Oligonucleotide
cccgtctatgtagcaaaata

SEQ ID NO: 87 - DNA, 20 bp, Artificial Sequence
  Antisense Oligonucleotide
acctaagaacagctaaaaga

SEQ ID NO: 88 - DNA, 20 bp, Artificial Sequence
  Antisense Oligonucleotide
taagacccccgaaaccagac

SEQ ID NO: 89 - DNA, 20 bp, Artificial Sequence
  Antisense Oligonucleotide
ataactttgcaaggagagcc

SEQ ID NO: 90 - DNA, 20 bp, Artificial Sequence
  Antisense Oligonucleotide
cttctgcataatgaattaac

SEQ ID NO: 91 - DNA, 20 bp, Artificial Sequence
  Antisense Oligonucleotide
atatagcaaggactaacccc

SEQ ID NO: 92 - DNA, 20 bp, Artificial Sequence
  Antisense Oligonucleotide
agatgaaaaattataaccaa

SEQ ID NO: 93 - DNA, 20 bp, Artificial Sequence
  Antisense Oligonucleotide
caatagatatagtaccgcaa

SEQ ID NO: 94 - DNA, 20 bp, Artificial Sequence
  Antisense Oligonucleotide
aggcgatagaaattgaaacc

SEQ ID NO: 95 - DNA, 20 bp, Artificial Sequence
  Antisense Oligonucleotide
tagccaaaccatttacccaa

SEQ ID NO: 96 - DNA, 20 bp, Artificial Sequence
  Antisense Oligonucleotide
caccttactaccagacaacc

SEQ ID NO: 97 - DNA, 19 bp, Artificial Sequence
  Antisense Oligonucleotide
ctaaacctagccccaaacc

SEQ ID NO: 98 - DNA, 20 bp, Artificial Sequence
  Antisense Oligonucleotide
ctagcatcaccagtattaga

SEQ ID NO: 99 - DNA, 20 bp, Artificial Sequence
  Antisense Oligonucleotide
ttaccaaaaacatcacctct

SEQ ID NO: 100 - DNA, 20 bp, Artificial Sequence
  Antisense Oligonucleotide
gaactcggcaaatcttaccc

SEQ ID NO: 101 - DNA, 20 bp, Artificial Sequence
  Sense Oligonucleotide
gggtaagatttgccgagttc

SEQ ID NO: 102 - DNA, 21 bp, Artificial Sequence
  Antisense Oligonucleotide
gctcataaggaaaggttaaaa

SEQ ID NO: 103 - DNA, 17 bp, Artificial Sequence
  Antisense Oligonucleotide
gtcaacccaacacaggc

SEQ ID NO: 104 - DNA, 21 bp, Artificial Sequence
  Antisense Oligonucleotide
accaacaagtcattattaccc

SEQ ID NO: 105 - DNA, 22 bp, Artificial Sequence
  Antisense Oligonucleotide
ggttgattgtagatattgggct

SEQ ID NO: 106 - DNA, 20 bp, Artificial Sequence
  Antisense Oligonucleotide
attaacagcccaatatctac

SEQ ID NO: 107 - DNA, 20 bp, Artificial Sequence
  Antisense Oligonucleotide
tgcgtcagattaaaacactg

SEQ ID NO: 108 - DNA, 20 bp, Artificial Sequence
  Antisense Oligonucleotide
aaaacattctcctccgcata

SEQ ID NO: 109 - DNA, 18 bp, Artificial Sequence
  Antisense Oligonucleotide
gttagtataagtaacatg

SEQ ID NO: 110 - DNA, 19 bp, Artificial Sequence
  Antisense Oligonucleotide
tggaccaatctatcaccct

SEQ ID NO: 111 - DNA, 20 bp, Artificial Sequence
  Antisense Oligonucleotide
acatataactgaactcctca

SEQ ID NO: 112 - DNA, 20 bp, Artificial Sequence
  Antisense Oligonucleotide
cacccactacctaaaaaatc

SEQ ID NO: 113 - DNA, 20 bp, Artificial Sequence
  Antisense Oligonucleotide
caccaattaagaaagcgttg

SEQ ID NO: 114 - DNA, 22 bp, Artificial Sequence
  Antisense Oligonucleotide
taggcctaaaagcagccaccaa

SEQ ID NO: 115 - DNA, 22 bp, Artificial Sequence
  Sense Oligonucleotide
ttggtggctgcttttaggccta

SEQ ID NO: 116 - DNA, 19 bp, Artificial Sequence
  Antisense Oligonucleotide
taacacccatagtaggcct

SEQ ID NO: 117 - DNA, 20 bp, Artificial Sequence
  Antisense Oligonucleotide
aaccttgtagagagagtaaa

SEQ ID NO: 118 - DNA, 20 bp, Artificial Sequence
  Antisense Oligonucleotide
aacagctctttggacactag

SEQ ID NO: 119 - DNA, 18 bp, Artificial Sequence
  Antisense Oligonucleotide
aactgttagtccaaagag

SEQ ID NO: 120 - DNA, 19 bp, Artificial Sequence
  Antisense Oligonucleotide
ctctaaatccccttgtaaa

SEQ ID NO: 121 - DNA, 19 bp, Artificial Sequence
  Antisense Oligonucleotide
actttaaatttgcccacag

SEQ ID NO: 122 - DNA, 19 bp, Artificial Sequence
  Antisense Oligonucleotide
ggttgtccaagatagaatc

SEQ ID NO: 123 - DNA, 19 bp, Artificial Sequence
  Antisense Oligonucleotide
acaaacctaccgagcctcc

SEQ ID NO: 124 - DNA, 18 bp, Artificial Sequence
  Antisense Oligonucleotide
atttataggttagaggcg

SEQ ID NO: 125 - DNA, 20 bp, Artificial Sequence
  Antisense Oligonucleotide
atgtagcaaaatagtgggaa

SEQ ID NO: 126 - DNA, 21 bp, Artificial Sequence
  Antisense Oligonucleotide
taagaacagctaaaagagcac

SEQ ID NO: 127 - DNA, 18 bp, Artificial Sequence
  Antisense Oligonucleotide
cgaaaccagacgagctac

SEQ ID NO: 128 - DNA, 22 bp, Artificial Sequence
  Sense Oligonucleotide
ggggtcttagctttggctctcc

SEQ ID NO: 129 - DNA, 20 bp, Artificial Sequence
  Antisense Oligonucleotide
taactttgcaaggagagcca

SEQ ID NO: 130 - DNA, 18 bp, Artificial Sequence
  Antisense Oligonucleotide
accttctgcataatgaat

SEQ ID NO: 131 - DNA, 19 bp, Artificial Sequence
  Antisense Oligonucleotide
atatagcaaggactaaccc

SEQ ID NO: 132 - DNA, 20 bp, Artificial Sequence
  Antisense Oligonucleotide
gatgaaaaattataaccaag

SEQ ID NO: 133 - DNA, 20 bp, Artificial Sequence
  Antisense Oligonucleotide
aatagatatagtaccgcaag

SEQ ID NO: 134 - DNA, 17 bp, Artificial Sequence
  Antisense Oligonucleotide
cgatagaaattgaaacc

SEQ ID NO: 135 - DNA, 20 bp, Artificial Sequence
  Sense Oligonucleotide
tactttatttgggtaaatgg

SEQ ID NO: 136 - DNA, 20 bp, Artificial Sequence
  Antisense Oligonucleotide
ccatttacccaaataaagta

SEQ ID NO: 137 - DNA, 20 bp, Artificial Sequence
  Antisense Oligonucleotide
ttagccaaaccatttaccca

SEQ ID NO: 138 - DNA, 21 bp, Artificial Sequence
  Antisense Oligonucleotide
aaggtggagtgggtttggggc

SEQ ID NO: 139 - DNA, 17 bp, Artificial Sequence
  Antisense Oligonucleotide
gctaaggttgtctggta

SEQ ID NO: 140 - DNA, 20 bp, Artificial Sequence
  Antisense Oligonucleotide
atcgcctatactttatttgg

SEQ ID NO: 141 - DNA, 20 bp, Artificial Sequence
  Antisense Oligonucleotide
atctattgcgccaggtttca

SEQ ID NO: 142 - DNA, 19 bp, Artificial Sequence
  Antisense Oligonucleotide
ttttcatctttcccttgcg

SEQ ID NO: 143 - DNA, 20 bp, Artificial Sequence
  Antisense Oligonucleotide
tccttgctatattatgcttg

SEQ ID NO: 144 - DNA, 20 bp, Artificial Sequence
  Antisense Oligonucleotide
cattatgcagaaggtatagg

SEQ ID NO: 145 - DNA, 18 bp, Artificial Sequence
  Antisense Oligonucleotide
tctccttgcaaagttatt

SEQ ID NO: 146 - DNA, 20 bp, Artificial Sequence
  Antisense Oligonucleotide
tttcgggggtcttagctttg

SEQ ID NO: 147 - DNA, 18 bp, Artificial Sequence
  Antisense Oligonucleotide
ctgttcttaggtagctcg

SEQ ID NO: 148 - DNA, 19 bp, Artificial Sequence
  Antisense Oligonucleotide
tgctacatagacgggtgtg

SEQ ID NO: 149 - DNA, 20 bp, Artificial Sequence
  Antisense Oligonucleotide
cctctacctataaatcttcc

SEQ ID NO: 150 - DNA, 17 bp, Artificial Sequence
  Antisense Oligonucleotide
gctatcaccaggctcgg

SEQ ID NO: 151 - DNA, 17 bp, Artificial Sequence
  Antisense Oligonucleotide
aagttgaactaagattc

SEQ ID NO: 152 - DNA, 20 bp, Artificial Sequence
  Antisense Oligonucleotide
gagggttctgtgggcaaatt

SEQ ID NO: 153 - DNA, 19 bp, Artificial Sequence
  Antisense Oligonucleotide
acagttaaatttacaaggg

SEQ ID NO: 154 - DNA, 19 bp, Artificial Sequence
  Antisense Oligonucleotide
gtgtccaaagagctgttcc

SEQ ID NO: 155 - DNA, 20 bp, Artificial Sequence
  Antisense Oligonucleotide
tactctctctacaaggtttt

SEQ ID NO: 156 - DNA, 20 bp, Artificial Sequence
  Antisense Oligonucleotide
taggcctactatgggtgtta

SEQ ID NO: 157 - DNA, 21 bp, Artificial Sequence
  Antisense Oligonucleotide
aacgctttcttaattggtggc

SEQ ID NO: 158 - DNA, 20 bp, Artificial Sequence
  Antisense Oligonucleotide
ttttaggtagtgggtgttga

SEQ ID NO: 159 - DNA, 20 bp, Artificial Sequence
  Antisense Oligonucleotide
ggagttcagttatatgtttg

SEQ ID NO: 160 - DNA, 20 bp, Artificial Sequence
  Antisense Oligonucleotide
tgatagattggtccaattgg

SEQ ID NO: 161 - DNA, 21 bp, Artificial Sequence
  Antisense Oligonucleotide
ctaacattagttcttctatag

SEQ ID NO: 162 - DNA, 18 bp, Artificial Sequence
  Antisense Oligonucleotide
atgcggaggagaatgttt

SEQ ID NO: 163 - DNA, 19 bp, Artificial Sequence
  Antisense Oligonucleotide
tcagtgttttaatctgacg

SEQ ID NO: 164 - DNA, 21 bp, Artificial Sequence
  Antisense Oligonucleotide
gtagatattgggctgttaatt

SEQ ID NO: 165 - DNA, 20 bp, Artificial Sequence
  Oligonucleotide
gtgagggtaataatgacttg

SEQ ID NO: 166 - DNA, 20 bp, Artificial Sequence
  Oligonucleotide
atgagcatgcctgtgttggt

SEQ ID NO: 167 - DNA, 19 bp, Artificial Sequence
  Oligonucleotide
ggtaagatttgccgagttc

SEQ ID NO: 168 - DNA, 20 bp, Artificial Sequence
  Oligonucleotide
tggtgatgctagaggtgatg

SEQ ID NO: 169 - DNA, 15 bp, Artificial Sequence
  Oligonucleotide
gcggtgcctctaata

SEQ ID NO: 170 - DNA, 20 bp, Artificial Sequence
  Oligonucleotide
ggccgttaaacatgtgtcac

SEQ ID NO: 171 - DNA, 20 bp, Artificial Sequence
  Oligonucleotide
tgattatgctacctttgcac

SEQ ID NO: 172 - DNA, 20 bp, Artificial Sequence
  Oligonucleotide
ttaaggaacaagtgattatg

SEQ ID NO: 173 - DNA, 20 bp, Artificial Sequence
  Oligonucleotide
tggagccattcatacaggtc

SEQ ID NO: 174 - DNA, 20 bp, Artificial Sequence
  Oligonucleotide
aaaagtaagagacagctgaa

SEQ ID NO: 175 - DNA, 20 bp, Artificial Sequence
  Oligonucleotide
cacgggcaggtcaatttcac

SEQ ID NO: 176 - DNA, 20 bp, Artificial Sequence
  Oligonucleotide
gtcttgctgtgttatgcccg

SEQ ID NO: 177 - DNA, 19 bp, Artificial Sequence
  Oligonucleotide
aattaaagctccatagggt

SEQ ID NO: 178 - DNA, 21 bp, Artificial Sequence
  Oligonucleotide
gtttgttaggtactgtttgca

SEQ ID NO: 179 - DNA, 19 bp, Artificial Sequence
  Oligonucleotide
aggtttggtagtttaggac

SEQ ID NO: 180 - DNA, 20 bp, Artificial Sequence
  Oligonucleotide
gccccaaccgaaatttttaa

SEQ ID NO: 181 - DNA, 20 bp, Artificial Sequence
  Oligonucleotide
ctcggaggttgggttctgct

SEQ ID NO: 182 - DNA, 20 bp, Artificial Sequence
  Oligonucleotide
ctggtgaagtcttagcatgt

SEQ ID NO: 183 - DNA, 20 bp, Artificial Sequence
  Oligonucleotide
caattgagtatagtagttcg

SEQ ID NO: 184 - DNA, 19 bp, Artificial Sequence
  Oligonucleotide
tgttccgttggtcaagtta

SEQ ID NO: 185 - DNA, 20 bp, Artificial Sequence
  Oligonucleotide
aataggattgcgctgttatc

SEQ ID NO: 186 - DNA, 20 bp, Artificial Sequence
  Oligonucleotide
attgttgatatggactctag

SEQ ID NO: 187 - DNA, 20 bp, Artificial Sequence
  Oligonucleotide
atccaacatcgaggtcgtaa

SEQ ID NO: 188 - DNA, 19 bp, Artificial Sequence
  Oligonucleotide
gcggctgcaccatcgggat

SEQ ID NO: 189 - DNA, 19 bp, Artificial Sequence
  Oligonucleotide
ttgaacaaacgaaccttta

SEQ ID NO: 190 - DNA, 20 bp, Artificial Sequence
  Oligonucleotide
aactcagatcacgtaggact

SEQ ID NO: 191 - DNA, 19 bp, Artificial Sequence
  Oligonucleotide
aaaccgacctggattactc

SEQ ID NO: 192 - DNA, 20 bp, Artificial Sequence
  Oligonucleotide
agggaggaatttgaaggtag

SEQ ID NO: 193 - DNA, 20 bp, Artificial Sequence
  Oligonucleotide
ggccttatttctcttgtcct

SEQ ID NO: 194 - DNA, 20 bp, Artificial Sequence
  Oligonucleotide
ggaaggcgctttgtgaagta

SEQ ID NO: 195 - DNA, 20 bp, Artificial Sequence
  Oligonucleotide
aagttgagatgatatcattt

SEQ ID NO: 196 - DNA, 17 bp, Artificial Sequence
  Oligonucleotide
cctgttcttgggtgggt

SEQ ID NO: 197 - DNA, 18 bp, Artificial Sequence
  Oligonucleotide
gtcctaaactaccaaacc

SEQ ID NO: 198 - DNA, 20 bp, Artificial Sequence
  Oligonucleotide
caccctctaacctagagaag

SEQ ID NO: 199 - DNA, 18 bp, Artificial Sequence
  Oligonucleotide
aggtggagtggattgggg

SEQ ID NO: 200 - RNA, 2374 nt, Homo sapiens
ucuaauacuggugaugcuagaggugauguuuuugguaaacaggcgggguaagauuugccgaguuccuuuuacuuuuuuuaaccuuuccuuaugagcaugccuguguuggguugacagugaggguaauaaugacuuguugguugauuguagauauugggcuguuaauugucaguucaguguuuuaaucugacgcaggcuuaugcggaggagaauguuuucauguuacuuauacuaacauuaguucuucuauagggugauagauugguccaauugggugugaggaguucaguuauauguuugggauuuuuuagguaguggguguugagcuugaacgcuuucuuaauugguggcugcuuuuaggccuacuauggguguuaaauuuuuuacucucucuacaagguuuuuuccuaguguccaaagagcuguuccucuuuggacuaacaguuaaauuuacaaggggauuuagaggguucugugggcaaauuuaaaguugaacuaagauucuaucuuggacaaccagcuaucaccaggcucgguagguuugucgccucuaccuauaaaucuucccacuauuuugcuacauagacgggugugcucuuuuagcuguucuuagguagcucgucugguuucgggggucuuagcuuuggcucuccuugcaaaguuauuucuaguuaauucauuaugcagaagguauagggguuaguccuugcuauauuaugcuugguuauaauuuuucaucuuucccuugcgguacuauaucuauugcgccagguuucaauuucuaucgccuauacuuuauuuggguaaaugguuuggcuaaaccuagccccaaacccacuccaccuuacuaccagacaaccuuagccaaaccauuuacccaaauaaaguauaggcgauagaaauugaaaccuggcgcaauagauauaguaccgcaagggaaagaugaaaaauuauaaccaagcauaauauagcaaggacuaaccccuauaccuucugcauaaugaauuaacuagaaauaacuuugcaaggagagccaaagcuaagacccccgaaaccagacgagcuaccuaagaacagcuaaaagagcacacccgucuauguagcaaaauagugggaagauuuauagguagaggcgacaaaccuaccgagccuggugauagcugguuguccaagauagaaucuuaguucaacuuuaaauuugcccacagaacccucuaaauccccuuguaaauuuaacuguuaguccaaagaggaacagcucuuuggacacuaggaaaaaaccuuguagagagaguaaaaaauuuaacacccauaguaggccuaaaagcagccaccaauuaagaaagcguucaagcucaacacccacuaccuaaaaaaucccaaacauauaacugaacuccucacacccaauuggaccaaucuaucacccuauagaagaacuaauguuaguauaaguaacaugaaaacauucuccuccgcauaagccugcgucagauuaaaacacugaacugacaauuaacagcccaauaucuacaaucaaccaacaagucauuauuacccucacugucaacccaacacaggcaugcucauaaggaaagguuaaaaaaaguaaaaggaacucggcaaaucuuaccccgccuguuuaccaaaaacaucaccucuagcaucaccaguauuagaggcaccgccugcccagugacacauguuuaacggccgcgguacccuaaccgugcaaagguagcauaaucacuuguuccuuaaauagggaccuguaugaauggcuccacgaggguucagcugucucuuacuuuuaaccagugaaauugaccugcccgugaagaggcgggcauaacacagcaagacgagaagacccuauggagcuuuaauuuauuaaugcaaacaguaccuaacaaacccacagguccuaaacuaccaaaccugcauuaaaaauuucgguuggggcgaccucggagcagaacccaaccuccgagcaguacaugcuaagacuucaccagucaaagcgaacuacuauacucaauugauccaauaacuugaccaacggaacaaguuacccuagggauaacagcgcaauccuauucuagaguccauaucaacaauaggguuuacgaccucgauguuggaucaggacaucccgauggugcagccgcuauuaaagguucguuuguucaacgauuaaaguccuacgugaucugaguucagaccggaguaauccaggucgguuucuaucuaccuucaaauuccucccuguacgaaaggacaagagaaauaaggccuacuucacaaagcgccuucccccguaaaugauaucaucucaacuuaguauuauacccacacccacccaagaacaggguuu

SEQ ID NO: 201 - RNA, 1679 nt, Homo sapiens
ggggucuuagcuuuggcucuccuugcaaaguuauuucuaguuaauucauuaugcagaagguauagggguuaguccuugcuauauuaugcuugguuauaauuuuucaucuuucccuugcggugcuaaaccuagccccaaacccacuccaccuuacuaccagacaaccuuagccaaaccauuuacccaaauaaaguauaggcgauagaaauugaaaccuggcgcaauagauauaguaccgcaagggaaagaugaaaaauuauaaccaagcauaauauagcaaggacuaaccccuauaccuucugcauaaugaauuaacuagaaauaacuuugcaaggagagccaaagcuaagacccccgaaaccagacgagcuaccuaagaacagcuaaaagagcacacccgucuauguagcaaaauagugggaagauuuauagguagaggcgacaaaccuaccgagccuggugauagcugguuguccaagauagaaucuuaguucaacuuuaaauuugcccacagaacccucuaaauccccuuguaaauuuaacuguuaguccaaagaggaacagcucuuuggacacuaggaaaaaaccuuguagagagaguaaaaaauuuaacacccauaguaggccuaaaagcagccaccaauuaagaaagcguucaagcucaacacccacuaccuaaaaaaucccaaacauauaacugaacuccucacacccaauuggaccaaucuaucacccuauagaagaacuaauguuaguauaaguaacaugaaaacauucuccuccgcauaagccugcgucagauuaaaacacugaacugacaauuaacagcccaauaucuacaaucaaccaacaagucauuauuacccucacugucaacccaacacaggcaugcucauaaggaaagguuaaaaaaaguaaaaggaacucggcaaaucuuaccccgccuguuuaccaaaaacaucaccucuagcaucaccaguauuagaggcaccgccugcccagugacacauguuuaacggccgcgguacccuaaccgugcaaagguagcauaaucacuuguuccuuaaauagggaccuguaugaauggcuccacgaggguucagcugucucuuacuuuuaaccagugaaauugaccugcccgugaagaggcgggcauaacacagcaagacgagaagacccuauggagcuuuaauuuauuaaugcaaacaguaccuaacaaacccacagguccuaaacuaccaaaccugcauuaaaaauuucgguuggggcgaccucggagcagaacccaaccuccgagcaguacaugcuaagacuucaccagucaaagcgaacuacuauacucaauugauccaauaacuugaccaacggaacaaguuacccuagggauaacagcgcaauccuauucuagaguccauaucaacaauaggguuuacgaccucgauguuggaucaggacaucccaauggugcagccgcuauuaaagguucguuuguucaacgauuaaaguccuacgugaucugaguucagaccggaguaauccaggucgguuucuaucuacuucaaauuccucccuguacgaaaggacaagagaaauaaggccuacuucacaaagcgccuucccccguaaaugauaucaucucaacuuaguauuauacccacacccacccaagaacaggguuu

SEQ ID NO: 202 - RNA, 1635 nt, Homo sapiens
ggggucuuagcuuuggcucuccuugcaaaguuauuucuaguuaauucauuaugcagaagguauagggguuaguccuugcuaaaccuagccccaaacccacuccaccuuacuaccagacaaccuuagccaaaccauuuacccaaauaaaguauaggcgauagaaauugaaaccuggcgcaauagauauaguaccgcaagggaaagaugaaaaauuauaaccaagcauaauauagcaaggacuaaccccuauaccuucugcauaaugaauuaacuagaaauaacuuugcaaggagagccaaagcuaagacccccgaaaccagacgagcuaccuaagaacagcuaaaagagcacacccgucuauguagcaaaauagugggaagauuuauagguagaggcgacaaaccuaccgagccuggugauagcugguuguccaagauagaaucuuaguucaacuuuaaauuugcccacagaacccucuaaauccccuuguaaauuuaacuguuaguccaaagaggaacagcucuuuggacacuaggaaaaaaccuuguagagagaguaaaaaauuuaacacccauaguaggccuaaaagcagccaccaauuaagaaagcguucaagcucaacacccacuaccuaaaaaaucccaaacauauaacugaacuccucacacccaauuggaccaaucuaucacccuauagaagaacuaauguuaguauaaguaacaugaaaacauucuccuccgcauaagccugcgucagauuaaaacacugaacugacaauuaacagcccaauaucuacaaucaaccaacaagucauuauuacccucacugucaacccaacacaggcaugcucauaaggaaagguuaaaaaaaguaaaaggaacucggcaaaucuuaccccgccuguuuaccaaaaacaucaccucuagcaucaccaguauuagaggcaccgccugcccagugacacauguuuaacggccgcgguacccuaaccgugcaaagguagcauaaucacuuguuccuuaaauagggaccuguaugaauggcuccacgaggguucagcugucucuuacuuuuaaccagugaaauugaccugcccgugaagaggcgggcauaacacagcaagacgagaagacccuauggagcuuuaauuuauuaaugcaaacaguaccuaacaaacccacagguccuaaacuaccaaaccugcauuaaaaauuucgguuggggcgaccucggagcagaacccaaccuccgagcaguacaugcuaagacuucaccagucaaagcgaacuacuauacucaauugauccaauaacuugaccaacggaacaaguuacccuagggauaacagcgcaauccuauucuagaguccauaucaacaauaggguuuacgaccucgauguuggaucaggacaucccaauggugcagccgcuauuaaagguucguuuguucaacgauuaaaguccuacgugaucugaguucagaccggaguaauccaggucgguuucuaucuacuucaaauuccucccuguacgaaaggacaagagaaauaaggccuacuucacaaagcgccuucccccguaaaugauaucaucucaacuuaguauuauacccacacccacccaagaacaggguuu

SEQ ID NO: 203 - RNA, 1921 nt, Homo sapiens
aaccuccgagcaguacaugcuaagacuucaccagucaaagcgaacuacuauacucaauugauccaauaacuugaccaacggaacaaguuacccuagggauaacagcgcaauccuauucuagaguccauaucaacaauaggguuuacgaccucgauguuggaucaggacaucccaauggugcagccgcuauuaaagguucguuuguucaacgauuaaaguccuacgugaucugaguucagaccggaguaauccaggucgguuucuaucuacuucaaauuccucccuguacgaaaggacaagagaaauaaggccuacuucacaaagcgccuucccccguaaaugauaucaucucaacuuaguauuauacccuguucuuggguggguguggguauaauacuaaguugagaugauaucauuuacgggggaaggcgcuuugugaaguaggccuuauuucucuuguccuuucguacagggaggaauuugaaguagauagaaaccgaccuggauuacuccggucugaacucagaucacguaggacuuuaaucguugaacaaacgaaccuuuaauagcggcugcaccaucgggauguccugauccaacaucgaggucguaaacccuauuguugauauggacucuagaauaggauugcgcuguuaucccuaggguaacuuguuccguuggucaaguuauuggaucaauugaguauaguaguucgcuuugacuggugaagucuuagcauguacugcucggagguuggguucugcuccgaggucgccccaaccgaaauuuuuaaugcagguuugguaguuuaggaccuguggguuuguuagguacuguuugcauuaauaaauuaaagcuccauagggucuucucgucuugcuguguuaugcccgccucuucacgggcaggucaauuucacugguuaaaaguaagagacagcugaacccucguggagccauucauacaggucccuauuuaaggaacaagugauuaugcuaccuuugcacgguuaggguaccgcggccguuaaacaugugucacugggcaggcggugccucuaauacuggugaugcuagaggugauguuuuugguaaacaggcgggguaagauuugccgaguuccuuuuacuuuuuuuaaccuuuccuuaugagcaugccuguguuggguugacagugaggguaauaaugacuuguugguugauuguagauauugggcuguuaauugucaguucaguguuuuaaucugacgcaggcuuaugcggaggagaauguuuucauguuacuuauacuaacauuaguucuucuauagggugauagauugguccaauugggugugaggaguucaguuauauguuugggauuuuuuagguaguggguguugagcuugaacgcuuucuuaauugguggcugcuuuuaggccuacuauggguguuaaauuuuuuacucucucuacaagguuuuuuccuaguguccaaagagcuguuccucuuuggacuaacaguuaaauuuacaaggggauuuagaggguucugugggcaaauuuaaaguugaacuaagauucuaucuuggacaaccagcuaucaccaggcucgguagguuugucgccucuaccuauaaaucuucccacuauuuugcuacauagacgggugugcucuuuuagcuguucuuagguagcucgucugguuucgggggucuuagcuuuggcucuccuugcaaaguuauuucuaguuaauucauuaugcagaagguauagggguuaguccuugcuauauuaugcuugguuauaauuuuucaucuuucccuugcgguacuauaucuauugcgccagguuucaauuucuaucgccuauacuuuauuuggguaaaugguuuggcuaagguugucugguaguaagguggaguggguuuggggcuagguuuagc

SEQ ID NO: 204 - RNA, 1744 nt, Homo sapiens
uagggauaacagcgcaauccuauucuagaguccauaucaacaauaggguuuacgaccucgauguuggaucaggacaucccgauggugcagccgcuauuaaagguucguuuguucaacgauuaaaguccuacgugaucugaguucagaccggaguaauccaggucgguuucuaucuaccuucaaauuccucccuguucuuggguggguguggguauaauacuaaguugagaugauaucauuuacgggggaaggcgcuuugugaaguaggccuuauuucucuuguccuuucguacagggaggaauuugaaguagauagaaaccgaccuggauuacuccggucugaacucagaucacguaggacuuuaaucguugaacaaacgaaccuuuaauagcggcugcaccaucgggauguccugauccaacaucgaggucguaaacccuauuguugauauggacucuagaauaggauugcgcuguuaucccuaggguaacuuguuccguuggucaaguuauuggaucaauugaguauaguaguucgcuuugacuggugaagucuuagcauguacugcucggagguuggguucugcuccgaggucgccccaaccgaaauuuuuaaugcagguuugguaguuuaggaccuguggguuuguuagguacuguuugcauuaauaaauuaaagcuccauagggucuucucgucuugcuguguuaugcccgccucuucacgggcaggucaauuucacugguuaaaaguaagagacagcugaacccucguggagccauucauacaggucccuauuuaaggaacaagugauuaugcuaccuuugcacgguuaggguaccgcggccguuaaacaugugucacugggcaggcggugccucuaauacuggugaugcuagaggugauguuuuugguaaacaggcgggguaagauuugccgaguuccuuuuacuuuuuuuaaccuuuccuuaugagcaugccuguguuggguugacagugaggguaauaaugacuuguugguugauuguagauauugggcuguuaauugucaguucaguguuuuaaucugacgcaggcuuaugcggaggagaauguuuucauguuacuuauacuaacauuaguucuucuauagggugauagauugguccaauugggugugaggaguucaguuauauguuugggauuuuuuagguaguggguguugagcuugaacgcuuucuuaauugguggcugcuuuuaggccuacuauggguguuaaauuuuuuacucucucuacaagguuuuuuccuaguguccaaagagcuguuccucuuuggacuaacaguuaaauuuacaaggggauuuagaggguucugugggcaaauuuaaaguugaacuaagauucuaucuuggacaaccagcuaucaccaggcucgguagguuugucgccucuaccuauaaaucuucccacuauuuugcuacauagacgggugugcucuuuuagcuguucuuagguagcucgucugguuucgggggucuuagcuuuggcucuccuugcaaaguuauuucuaguuaauucauuaugcagaagguauagggguuaguccuugcuauauuaugcuugguuauaauuuuucaucuuucccuugcgguacuauaucuauugcgccagguuucaauuucuaucgccuauacuuuauuuggguaaaugguuuggcuaagguugucugguaguaagguggaguggguuuggggcuagguuuagc

SEQ ID NO: 205 - RNA, 1854 nt, Homo sapiens
gaacucggcaaaucuuaccccgccuguuuaccaaaaacaucaccucuagcaucaccaguauuagaggcaccgccugcccagugacacauguuuaacggccgcgguacccuaaccgugcaaagguagcauaaucacuuguuccuuaaauagggaccuguaugaauggcuccacgaggguucagcugucucuuacuuuuaaccagugaaauugaccugcccgugaagaggcgggcaugacacagcaagacgagaagacccuauggagcuuuaauuuauuaaugcaaacaguaccuaacaaacccuguucuuggguggguguggguauaauacuaaguugagaugauaucauuuacgggggaaggcgcuuugugaaguaggccuuauuucucuuguccuuucguacagggaggaauuugaaguagauagaaaccgaccuggauuacuccggucugaacucagaucacguaggacuuuaaucguugaacaaacgaaccuuuaauagcggcugcaccaucgggauguccugauccaacaucgaggucguaaacccuauuguugauauggacucuagaauaggauugcgcuguuaucccuaggguaacuuguuccguuggucaaguuauuggaucaauugaguauaguaguucgcuuugacuggugaagucuuagcauguacugcucggagguuggguucugcuccgaggucgccccaaccgaaauuuuuaaugcagguuugguaguuuaggaccuguggguuuguuagguacuguuugcauuaauaaauuaaagcuccauagggucuucucgucuugcuguguuaugcccgccucuucacgggcaggucaauuucacugguuaaaaguaagagacagcugaacccucguggagccauucauacaggucccuauuuaaggaacaagugauuaugcuaccuuugcacgguuaggguaccgcggccguuaaacaugugucacugggcaggcggugccucuaauacuggugaugcuagaggugauguuuuugguaaacaggcgggguaagauuugccgaguuccuuuuacuuuuuuuaaccuuuccuuaugagcaugccuguguuggguugacagugaggguaauaaugacuuguugguugauuguagauauugggcuguuaauugucaguucaguguuuuaaucugacgcaggcuuaugcggaggagaauguuuucauguuacuuauacuaacauuaguucuucuauagggugauagauugguccaauugggugugaggaguucaguuauauguuugggauuuuuuagguaguggguguugagcuugaacgcuuucuuaauugguggcugcuuuuaggccuacuauggguguuaaauuuuuuacucucucuacaagguuuuuuccuaguguccaaagagcuguuccucuuuggacuaacaguuaaauuuacaaggggauuuagaggguucugugggcaaauuuaaaguugaacuaagauucuaucuuggacaaccagcuaucaccaggcucgguagguuugucgccucuaccuauaaaucuucccacuauuuugcuacauagacgggugugcucuuuuagcuguucuuagguagcucgucugguuucgggggucuuagcuuuggcucuccuugcaaaguuauuucuaguuaauucauuaugcagaagguauagggguuaguccuugcuauauuaugcuugguuauaauuuuucaucuuucccuugcgguacuauaucuauugcgccagguuucaauuucuaucgccuauacuuuauuuggguaaaugguuuggcuaagguugucugguaguaagguggaguggguuuggggcuagguuuagc